(19) United States
(12) Patent Application Publication
(10) Pub. No.: US 2017/0233345 A1
(43) Pub. Date: Aug. 17, 2017

(54) SUBSTITUTED QUINOLINE-4-CARBOXAMIDES AND USE THEREOF

(71) Applicant: BAYER PHARMA AKTIENGESELLSCHAFT, Berlin (DE)

(72) Inventors: Alexandros VAKALOPOULOS, Hilden (DE); Gaelle VALOT, Wuppertal (DE); Niels LINDNER, Wuppertal (DE); Markus FOLLMANN, Köln (DE); Johannes-Peter STASCH, Grottaferrata (RM) (IT); Frank WUNDER, Wuppertal (DE); Tobias MARQUARDT, Wuppertal (DE); Lisa DIETZ, Wuppertal (DE); Volkhart Min-Jian LI, Velbert (DE)

(73) Assignee: BAYER PHARMA AKTIENGESELLSCHAFT, Berlin (DE)

(21) Appl. No.: 15/503,243

(22) PCT Filed: Aug. 11, 2015
(86) PCT No.: PCT/EP2015/068418

§ 371 Date: Feb. 10, 2017

(30) Foreign Application Priority Data
Aug. 14, 2014 (EP) 14181030.9

(51) Int. Cl. C07D 215/48; A61K 31/47; A61K 31/4709; C07D 491/052; C07D 413/12; C07D 405/12; C07D 417/12; C07D 451/14; A61K 45/06; C07D 401/12

(52) U.S. Cl. CPC C07D 215/48; A61K 45/06; A61K 31/47; A61K 31/4709; C07D 401/12; C07D 413/12; C07D 405/12; C07D 417/12; C07D 451/14; C07D 491/052

(57) ABSTRACT

The present application relates to novel substituted quinoline-4-carboxamides and use thereof, to processes for their preparation, to their use, alone or in combinations, for the treatment and/or prophylaxis of diseases, and to the use thereof for production of medicaments for the treatment and/or prophylaxis of diseases, especially for the treatment and/or prophylaxis of cardiovascular disorders.

The present application relates to novel substituted quinoline-4-carboxamides and use thereof, to processes for their preparation, to their use, alone or in combinations, for the treatment and/or prophylaxis of diseases, and to the use thereof for production of medicaments for the treatment and/or prophylaxis of diseases, especially for the treatment and/or prophylaxis of cardiovascular disorders.

One of the most important cellular transmission systems in mammalian cells is cyclic guanosine monophosphate (cGMP). Together with nitrogen monoxide (NO), which is released from the endothelium and transmits hormonal and mechanical signals, it forms the NO/cGMP system. Guanylate cyclases catalyze the biosynthesis of cGMP from guanosine triphosphate (GTP). The representatives of this family known to date can be classified into two groups either by structural features or by the type of ligands: the particulate guanylate cyclases which can be stimulated by natriuretic peptides, and the soluble guanylate cyclases which can be stimulated by NO. The soluble guanylate cyclases consist of two subunits and very probably contain one heme per heterodimer, which is part of the regulatory center. This is of central importance for the activation mechanism. NO is able to bind to the iron atom of heme and thus markedly increase the activity of the enzyme. heme-free preparations cannot, by contrast, be stimulated by NO. Carbon monoxide (CO) is also able to bind to the central iron atom of heme, but the stimulation by CO is much less than that by NO.

By forming cGMP, and owing to the resulting regulation of phosphodiesterases, ion channels and protein kinases, guanylate cyclase plays an important role in various physiological processes, in particular in the relaxation and proliferation of smooth muscle cells, in platelet aggregation and platelet adhesion and in neuronal signal transmission, and also in disorders which are based on a disruption of the aforementioned processes. Under pathophysiological conditions, the NO/cGMP system can be suppressed, which can lead, for example, to hypertension, platelet activation, increased cell proliferation, endothelial dysfunction, atherosclerosis, angina pectoris, heart failure, myocardial infarction, thromboses, stroke and sexual dysfunction.

Owing to the expected high efficiency and low level of side effects, a possible NO-independent treatment for such disorders by targeting the influence of the cGMP signal pathway in organisms is a promising approach.

Hitherto, for the therapeutic stimulation of the soluble guanylate cyclase, use has exclusively been made of compounds such as organic nitrates whose effect is based on NO. The latter is formed by bioconversion and activates soluble guanylate cyclase by attacking the central iron atom of heme.

In addition to the side effects, the development of tolerance is one of the crucial disadvantages of this mode of treatment.

In recent years, some substances have been described which stimulate soluble guanylate cyclase directly, i.e. without prior release of NO, such as, for example, 3-(5′-hydroxymethyl-2′-furyl)-1-benzylindazole [YC-1; Wu et al., Blood 84 (1994), 4226; Mülsch et al., Brit. J. Pharmacol. 120 (1997), 681], fatty acids [Goldberg et al., J. Biol. Chem. 252 (1977), 1279], diphenyliodonium hexafluorophosphate [Pettibone et al., Eur. J. Pharmacol. 116 (1985), 307], isoliquiritigenin [Yu et al., Brit. J. Pharmacol. 114 (1995), 1587] and various substituted pyrazole derivatives (WO 98/16223).

Quinoline derivatives which can be used for treating disorders are described, inter alia, in EP 0808628 as bradykinin antagonists, in WO 00/42026 as GLP-1 agonists and in WO 2006/069656 as CXCR-2 inhibitors.

It was an object of the present invention to provide novel substances which act as stimulators of soluble guanylate cyclase and are suitable as such for the treatment and/or prophylaxis of diseases.

The present invention provides compounds of the general formulae (I-A) and (I-B)

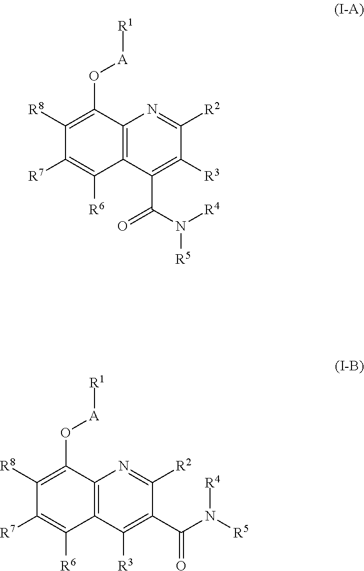

in which
A represents CH₂, CD₂ or CH(CH₃),
R¹ represents (C₄-C₆)-alkyl, (C₃-C₇)-cycloalkyl, pyridyl or phenyl,
   where (C₄-C₇)-alkyl may be up to hexasubstituted by fluorine,
   where (C₃-C₇)-cycloalkyl may be substituted by 1 to 4 substituents independently of one another selected from the group consisting of fluorine, trifluoromethyl and (C₁-C₄)-alkyl,
   where pyridyl is substituted by 1 or 2 substituents independently of one another selected from the group consisting of halogen, cyano and (C₁-C₄)-alkyl,
   and
   where phenyl may be substituted by 1 to 4 substituents independently of one another selected from the group consisting of halogen, cyano, monofluoromethyl, difluoromethyl, trifluoromethyl, (C₁-C₄)-alkyl, (C₂-C₃)-alkynyl, (C₁-C₄)-alkoxy, (C₃-C₅)-cyclopropyl, difluoromethoxy and trifluoromethoxy,
R² represents hydrogen, halogen, (C₁-C₄)-alkyl, (C₁-C₄)-alkoxy, cyclopropyl, monofluoromethyl, difluoromethyl or trifluoromethyl,
R³ represents hydrogen, halogen, (C₁-C₄)-alkyl, (C₁-C₄)-alkoxy, cyclopropyl, monofluoromethyl, difluoromethyl or trifluoromethyl,
R⁴ represents hydrogen or (C₁-C₄)-alkyl,
R⁵ is a group of the formula

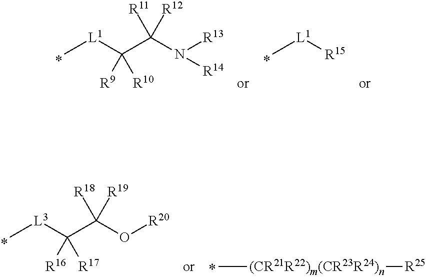

where
* represents the point of attachment to the amino group,
L¹ represents a bond, methanediyl or 1,2-ethanediyl,
   in which methanediyl and 1,2-ethanediyl may be substituted by 1 or 2 substituents independently of one another selected from the group consisting of fluorine, trifluoromethyl, (C₁-C₄)-alkyl, (C₃-C₅)-cycloalkyl, hydroxy and (C₁-C₄)-alkoxy,
L² represents a bond or (C₁-C₄)-alkanediyl,
   in which (C₁-C₄)-alkanediyl may be substituted by 1 to 3 substituents independently of one another selected from the group consisting of fluorine, trifluoromethyl, (C₁-C₄)-alkyl, (C₃-C₅)-cycloalkyl, hydroxy and (C₁-C₄)-alkoxy,
L³ represents a bond, methanediyl or 1,2-ethanediyl,
   in which methanediyl or 1,2-ethanediyl may be substituted by 1 or 2 substituents independently of one another selected from the group consisting of fluorine, trifluoromethyl, (C₁-C₄)-alkyl, (C₃-C₇)-cycloalkyl, hydroxy and (C₁-C₄)-alkoxy,
R⁹ represents hydrogen, (C₁-C₆)-alkyl, (C₂-C₆)-alkenyl, (C₂-C₆)-alkynyl, (C₃-C₇)-cycloalkyl, 5- or 6-membered heteroaryl or phenyl,
   in which (C₁-C₆)-alkyl may be substituted by 1 or 2 substituents independently of one another selected from the group consisting of trifluoromethyl, difluoromethoxy, trifluoromethoxy, hydroxy, (C₁-C₄)-alkoxy, (C₁-C₄)-alkoxycarbonyl, (C₁-C₄)-alkylthio, (C₁-C₄)-alkylsulfonyl, phenyl, phenoxy and benzyloxy, and up to hexasubstituted by fluorine,
   in which phenyl, phenoxy and benzyloxy may be substituted by 1 to 3 halogen substituents,
   in which (C₃-C₇)-cycloalkyl may be substituted by 1 or 2 substituents independently of one another selected from the group consisting of fluorine, trifluoromethyl, (C₁-C₄)-alkyl and (C₁-C₄)-alkoxy,
   and
   in which phenyl and 5- or 6-membered heteroaryl may be substituted by 1 to 3 substituents independently of one another selected from the group consisting of halogen, cyano, nitro, trifluoromethyl, (C₁-C₄)-alkyl, (C₁-C₄)-alkoxy, difluoromethoxy, trifluoromethoxy and (C₁-C₄)-alkylsulfonyl,
R¹⁰ represents hydrogen or (C₁-C₆)-alkyl,
or
R⁹ and R¹⁰ together with the carbon atom to which they are attached form a 3- to 7-membered carbocycle or a 4- to 7-membered heterocycle,
   in which the 3- to 7-membered carbocycle and the 4- to 7-membered heterocycle for their part may be substituted by 1 or 2 substituents independently of one another selected from the group consisting of fluorine and (C₁-C₄)-alkyl,
R¹¹ represents hydrogen, (C₁-C₁₀)-alkyl, (C₃-C₇)-cycloalkyl, (C₂-C₆)-alkenyl, (C₂-C₆)-alkynyl, 5- or 6-membered heteroaryl or phenyl,
   in which (C₁-C₁₀)-alkyl may be substituted by 1 or 2 substituents independently of one another selected from the group consisting of trifluoromethyl, difluoromethoxy, trifluoromethoxy, hydroxy, (C₁-C₄)-alkoxy, benzyloxy, phenoxy and phenyl, and up to hexasubstituted by fluorine,
      in which benzyloxy, phenoxy and phenyl may be substituted by 1 to 3 halogen or (C₁-C₄)-alkoxy substituents,
   in which (C₃-C₇)-cycloalkyl may be substituted by 1 or 2 fluorine or (C₁-C₄)-alkyl substituents,
   and
   in which phenyl and 5- or 6-membered heteroaryl may be substituted by 1 to 3 substituents independently of one another selected from the group consisting of halogen, cyano, trifluoromethyl, (C₁-C₄)-alkyl, (C₁-C₄)-alkoxy and (C₁-C₄)-alkylsulfonyl,
R¹² represents hydrogen or (C₁-C₆)-alkyl,
or
R¹¹ and R¹² together with the carbon atom to which they are attached form a 3- to 7-membered carbocycle or a 4- to 7-membered heterocycle,
   in which the 3- to 7-membered carbocycle and the 4- to 7-membered heterocycle for their part may be substituted by 1 or 2 substituents independently of one another selected from the group consisting of fluorine and (C₁-C₄)-alkyl,
or
R⁹ and R¹¹ together with the carbon atoms to which they are attached form a 3- to 7-membered carbocycle or a 4- to 7-membered heterocycle,
with the proviso that not more than one of the R⁹ and R¹⁰, R¹¹ and R¹², and R⁹ and R¹¹ radical pairs at the same time forms a carbo- or heterocycle,
with the proviso that the R⁹ and R¹¹ radicals do not both simultaneously represent phenyl or 5- or 6-membered heteroaryl,
R¹³ represents hydrogen or (C₁-C₄)-alkyl,
   in which (C₁-C₄)-alkyl may be substituted by 1 to 3 substituents independently of one another selected from the group consisting of fluorine, trifluoromethyl, hydroxy and (C₁-C₄)-alkoxy,
R¹⁴ represents hydrogen, (C₁-C₆)-alkyl, (C₃-C₇)-cycloalkyl, phenyl or benzyl,
   in which (C₁-C₆)-alkyl may be substituted by 1 to 3 substituents independently of one another selected from the group consisting of fluorine, trifluoromethyl, hydroxy, (C₁-C₄)-alkoxy and phenoxy,
   and
   in which phenyl and benzyl may be substituted by 1 to 3 substituents independently of one another selected from the group consisting of halogen and trifluoromethyl,
or
R¹³ and R¹⁴ together with the nitrogen atom to which they are attached form a 4- to 7-membered azaheterocycle,
R¹⁵ represents 5- to 10-membered azaheterocyclyl attached via a ring carbon atom,
   in which 5- to 10-membered azaheterocyclyl attached via a ring carbon atom may be substituted by 1 to 2 trifluoromethyl, (C₃-C₇)-cycloalkyl, oxo and benzyl substituents, and up to four times by (C₁-C₄)-alkyl and up to twice by fluorine,
   in which 5- to 10-membered azaheterocyclyl may be fused to a phenyl ring or a pyridyl ring, which for their part may be substituted by 1 or 2 substituents independently of one another selected from the group consisting of halogen, (C₁-C₄)-alkyl, (C₁-C₄)-alkoxy and trifluoromethyl,
R¹⁶ represents hydrogen, (C₁-C₁₀)-alkyl, (C₂-C₆)-alkenyl, (C₂-Cₒ)-alkynyl, (C₃-C₇)-cycloalkyl, (C₁-C₄)-alkoxycarbonyl, —(C═O)NR²⁶R²⁷, 5- or 6-membered heteroaryl or phenyl,
   in which (C₁-C₁₀)-alkyl may be substituted by 1 or 2 substituents independently of one another selected from the group consisting of difluoromethoxy, trifluoromethoxy, hydroxy, (C₁-C₄)-alkoxy, (C₁-C₄)-alkoxycarbonyl, (C₁-C₄)-alkylthio, (C₁-C₄)-alkylsulfonyl, phenyl, phenoxy and benzyloxy, and up to hexasubstituted by fluorine,
      in which phenyl, phenoxy and benzyloxy for their part may be substituted by 1 to 3 halogen substituents,
   in which (C₃-C₇)-cycloalkyl may be substituted by 1 or 2 substituents independently of one another selected from the group consisting of fluorine, trifluoromethyl, (C₁-C₄)-alkyl and (C₁-C₄)-alkoxy,
   in which R²⁶ represents hydrogen, (C₁-C₄)-alkyl, (C₃-C₇)-cycloalkyl, aryl or naphthyl,
   in which R²⁷ represents hydrogen or methyl,
   and
   in which phenyl and 5- or 6-membered heteroaryl may be substituted by 1 to 3 substituents independently of one another selected from the group consisting of halogen, cyano, trifluoromethyl, difluoromethyl, (C₁-C₄)-alkyl, (C₁-C₄)-alkoxy and (C₁-C₄)-alkylsulfonyl,
      in which (C₁-C₄)-alkyl may be substituted by amino or hydroxy,
R¹⁷ represents hydrogen or (C₁-C₆)-alkyl,
   in which (C₁-C₄)-alkyl may be substituted by hydroxy,
or
R¹⁶ and R⁷ together with the carbon atom to which they are attached form a 3- to 7-membered carbocycle or a 4- to 7-membered heterocycle,
   in which the 3- to 7-membered carbocycle and the 4- to 7-membered heterocycle for their part may be substituted by 1 or 2 substituents independently of one another selected from the group consisting of fluorine and (C₁-C₄)-alkyl,
R¹⁸ represents hydrogen, (C₁-C₆)-alkyl, (C₂-C₆)-alkenyl, (C₂-C₆)-alkynyl, (C₃-C₇)-cycloalkyl, (C₁-C₄)-alkoxycarbonyl, 5- or 6-membered heteroaryl or phenyl,
   in which (C₁-C₆)-alkyl may be substituted by 1 or 2 substituents independently of one another selected from the group consisting of trifluoromethyl, difluoromethoxy, trifluoromethoxy, hydroxy, (C₁-C₄)-alkoxy, (C₁-C₄)-alkoxycarbonyl, (C₁-C₄)-alkylthio, (C₁-C₄)-alkylsulfonyl, phenyl, phenoxy and benzyloxy, and up to hexasubstituted by fluorine,
      in which phenyl, phenoxy and benzyloxy for their part may be substituted by 1 to 3 halogen substituents,
   in which (C₃-C₇)-cycloalkyl may be substituted by 1 or 2 substituents independently of one another selected from the group consisting of fluorine, trifluoromethyl, (C₁-C₄)-alkyl and (C₁-C₄)-alkoxy,
   and
   in which phenyl and 5- or 6-membered heteroaryl may be substituted by 1 to 3 substituents independently of one another selected from the group consisting of halogen, cyano, trifluoromethyl, (C₁-C₄)-alkyl, (C₁-C₄)-alkoxy and (C₁-C₄)-alkylsulfonyl,
R¹⁹ represents hydrogen or (C₁-C₆)-alkyl,
   in which (C₁-C₄)-alkyl may be substituted by hydroxy,
or
R¹⁸ and R¹⁹ together with the carbon atom to which they are attached form a 3- to 7-membered carbocycle or a 4- to 7-membered heterocycle,
   in which the 3- to 7-membered carbocycle and the 4- to 7-membered heterocycle for their part may be substituted by 1 or 2 substituents independently of one another selected from the group consisting of fluorine and (C₁-C₄)-alkyl,
with the proviso that the R¹⁶ and R¹⁸ radicals are not both simultaneously phenyl or 5- or 6-membered heteroaryl,
or
R¹⁶ and R¹⁸ together with the carbon atoms to which they are attached form a 3- to 7-membered carbocycle or a 4- to 7-membered heterocycle,
with the proviso that not more than one of the R¹⁶ and R¹⁷, R¹⁸ and R¹⁹, and R¹⁶ and R¹⁸ radical pairs at the same time forms a carbo- or heterocycle,
R²⁰ represents hydrogen or (C₁-C₄)-alkyl,
   in which (C₁-C₄)-alkyl may be substituted by 1 to 5 fluorine substituents,
m represents 0, 1 or 2,
n represents 0 or 1,
R²¹ represents hydrogen, cyano or (C₁-C₆)-alkyl,
   in which (C₁-C₆)-alkyl may be substituted by 1 to 5 fluorine substituents,
R²² represents hydrogen or (C₁-C₄)-alkyl,
   in which (C₁-C₄)-alkyl may be substituted by 1 to 5 fluorine substituents,
R²³ represents hydrogen or (C₁-C₆)-alkyl,
   in which (C₁-C₆)-alkyl may be substituted by 1 to 5 fluorine substituents,
R²⁴ represents hydrogen or (C₁-C₄)-alkyl,
   in which (C₁-C₄)-alkyl may be substituted by 1 to 5 fluorine substituents,
or
R²¹ and R²² together with the carbon atom to which they are attached form a 3- to 7-membered carbocycle or a 4- to 7-membered heterocycle,
   in which the 3- to 7-membered carbocycle and the 4- to 7-membered heterocycle for their part may be substituted by 1 or 2 substituents independently of one another selected from the group consisting of fluorine and (C₁-C₄)-alkyl,
or
R²³ and R²⁴ together with the carbon atom to which they are attached form a 3- to 7-membered carbocycle or a 4- to 7-membered heterocycle,
   in which the 3- to 7-membered carbocycle and the 4- to 7-membered heterocycle for their part may be substituted by 1 or 2 substituents independently of one another selected from the group consisting of fluorine and (C₁-C₄)-alkyl,
or
R²¹ and R²³ together with the carbon atom to which they are attached form a 3- to 7-membered carbocycle or a 4- to 7-membered heterocycle,
   in which the 3- to 7-membered carbocycle and the 4- to 7-membered heterocycle for their part may be substituted by 1 or 2 substituents independently of one another selected from the group consisting of fluorine and (C₁-C₄)-alkyl,
with the proviso that not more than one of the R²¹ and R²², R²³ and R²⁴, and R²¹ and R²³ radical pairs at the same time forms a carbo- or heterocycle,
R²⁵ represents (C₁-C₆)-alkyl, (C₁-C₆)-alkoxy, (C₁-C₄)-alkoxycarbonyl, hydroxycarbonyl, aminocarbonyl, aminosulfonyl, 5- to 10-membered heterocyclyl attached via a ring carbon atom, 5- to 10-membered carbocyclyl, phenyl or 5- to 10-membered heteroaryl,
   in which (C₁-C₆)-alkyl may be substituted by cyano and up to hexasubstituted by fluorine,
   in which (C₁-C₆)-alkoxy may be substituted by hydroxy, amino, monoalkylamino, dialkylamino, cyclopropyl, phenyl or (C₂-C₄)-alkenyl,
   in which aminocarbonyl may be substituted by (C₁-C₆)-alkyl or (C₃-C₆)-cycloalkyl,
   in which aminosulfonyl may be substituted by (C₁-C₆)-alkyl or (C₃-C₆)-cycloalkyl,
   in which phenyl may be substituted by 1 to 3 substituents independently of one another selected from the group consisting of halogen, cyano, trifluoromethyl, difluoromethyl, (C₁-C₆)-alkyl, (C₁-C₄)-alkylcarbonyl, (C₁-C₄)-alkoxycarbonyl, hydroxycarbonyl, —(C═O)NR²⁸R²⁹, (C₁-C₄)-alkylsulfonyl, (C₃-C₆)-cycloalkylsulfonyl, (C₁-C₄)-alkylthio, (C₁-C₄)-alkoxy, trifluoromethoxy, difluoromethoxy, phenoxy, hydroxyl, 5- to 10-membered heteroaryl, 4- to 7-membered heterocyclyl and (C₃-C₇)-cycloalkyl,
      in which (C₁-C₆)-alkyl may be substituted by 1 or 2 substituents independently of one another selected from the group consisting of fluorine, trifluoromethoxy, (C₁-C₄)-alkylcarbonyl, —(C═O)NR²⁸R²⁹, (C₁-C₄)-alkoxy, (C₃-C₆)-cycloalkyl, morpholinyl, piperidinyl, pyrrolidinyl, piperazinyl, phenyl, hydroxy and amino,
         in which phenyl may be substituted by 1 to 3 halogen substituents,
         in which amino may be substituted by 1 or 2 substituents independently of one another selected from the group consisting of (C₁-C₆)-alkyl, (C₁-C₄)-alkylcarbonyl, (C₃-C₆)-cycloalkylsulfonyl, (C₁-C₄)-alkylsulfonyl and methoxy-(C₁-C₄)-alkyl,
         in which (C₃-C₆)-cycloalkyl may be substituted by amino or hydroxy,
         and in which
         R²⁸ and R²⁹ each independently of one another represent hydrogen, (C₁-C₄)-alkyl or (C₃-C₇)-cycloalkyl,
   in which 5- to 10-membered heteroaryl may be substituted by 1 to 3 substituents independently of one another selected from the group consisting of fluorine, chlorine, cyano, (C₁-C₆)-alkyl, trifluoromethyl, (C₁-C₄)-alkoxy, amino, (C₁-C₄)-alkoxycarbonyl, hydroxycarbonyl, —(C═O)NR²⁸R²⁹, phenyl, pyridyl, pyrimidyl, 1,3-thiazol-5-yl and (C₃-C₇)-cycloalkyl,
      in which (C₁-C₆)-alkyl may be substituted by 1 to 3 substituents independently of one another selected from the group consisting of halogen, cyano, hydroxy, amino, trifluoromethyl, difluoromethyl, alkylsulfonyl, (C₁-C₄)-alkylcarbonyl, (C₁-C₄)-alkoxycarbonyl, hydroxycarbonyl, (C₁-C₄)-alkylthio, (C₁-C₄)-alkoxy, trifluoromethoxy, difluoromethoxy, phenoxy, phenyl, pyridyl, pyrimidyl, 5-membered heteroaryl, tetrahydrothiophenyl 1,1-dioxide, (C₃-C₇)-cycloalkyl, morpholinyl, piperidinyl, pyrrolidinyl, 2-oxopyrrolidin-1-yl, piperazinyl, tetrahydrothiophenyl 1,1-dioxide, thiomorpholinyl 1,1-dioxide and azetidine,
         in which 5-membered heteroaryl may be substituted by 1 to 3 substituents independently of one another selected from the group consisting of halogen, (C₁-C₄)-alkyl and (C₁-C₄)-alkoxy,
         in which piperidinyl may be substituted by 1 to 4 fluorine substituents,
         in which phenyl may be substituted by 1 to 3 substituents independently of one another selected from the group consisting of halogen, (C₁-C₄)-alkyl and (C₁-C₄)-alkoxy,
         in which azetidine may be substituted by hydroxy,
         in which piperazinyl may be substituted by 1 to 3 substituents independently of one another selected from the group consisting of (C₁-C₄)-alkyl, (C₃-C₇)-cycloalkyl and trifluoromethyl,
      and in which
      R²⁸ and R²⁹ each independently of one another represent hydrogen, (C₁-C₄)-alkyl or (C₃-C₇)-cycloalkyl,
   in which 5- to 10-membered heterocyclyl attached via a ring carbon atom may be substituted by 1 to 3 substituents independently of one another selected from the group consisting of oxo, fluorine, trifluoromethyl, hydroxy and (C₁-C₄)-alkyl,
   in which 5- to 10-membered heterocyclyl attached via a ring carbon atom may be fused to a phenyl ring or a pyridyl ring, which for their part may be substituted by 1 to 3 substituents selected from the group consisting of halogen, (C₁-C₄)-alkyl and trifluoromethyl,
   and
   in which 5- to 10-membered carbocyclyl may be substituted by 1 to 3 substituents independently of one another selected from the group consisting of trifluoromethyl, fluorine, cyano, hydroxy, hydroxycarbonyl, (C₁-C₄)-alkoxycarbonyl, amino and (C₁-C₄)-alkyl,
      in which (C₁-C₄)-alkyl may be substituted by hydroxy or hydroxycarbonyl,
   in which 5- to 10-membered carbocyclyl may be fused to a phenyl ring or a pyridyl ring, which for its part may be substituted by 1 to 3 substituents selected from the group consisting of halogen, (C₁-C₄)-alkyl, (C₁-C₄)-alkoxy and trifluoromethyl,
R⁶ represents hydrogen,
R⁷ represents hydrogen, halogen, cyano, difluoromethyl, trifluoromethyl, (C₁-C₄)-alkyl, (C₃-C₇)-cycloalkyl, (C₂-C₄)-alkynyl, (C₁-C₄)-alkylamino, difluoromethoxy, trifluoromethoxy, (C₁-C₄)-alkoxy, amino, 4- to 7-membered heterocyclyl or 5- or 6-membered heteroaryl,
R⁸ represents hydrogen, cyano or halogen,

and the N-oxides, salts, solvates, salts of the N-oxides and solvates of the N-oxides and salts thereof.

Compounds of the invention are the compounds of the formula (I) and the salts, solvates and solvates of the salts thereof, the compounds that are encompassed by formula (I) and are of the formulae mentioned below and the salts, solvates and solvates of the salts thereof and the compounds that are encompassed by formula (I) and are cited below as working examples and the salts, solvates and solvates of the salts thereof if the compounds that are encompassed by formula (I) and are mentioned below are not already salts, solvates and solvates of the salts.

Preferred salts in the context of the present invention are physiologically acceptable salts of the compounds of the invention. Also encompassed are salts which are not themselves suitable for pharmaceutical applications but can be used, for example, for isolation or purification of the compounds according to the invention.

Physiologically acceptable salts of the compounds of the invention include acid addition salts of mineral acids, carboxylic acids and sulfonic acids, for example salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, formic acid, acetic acid, trifluoroacetic acid, propionic acid, lactic acid, tartaric acid, malic acid, citric acid, fumaric acid, maleic acid and benzoic acid.

Physiologically acceptable salts of the compounds of the invention also include salts of conventional bases, by way of example and with preference alkali metal salts (e.g. sodium and potassium salts), alkaline earth metal salts (e.g. calcium and magnesium salts) and ammonium salts derived from ammonia or organic amines having 1 to 16 carbon atoms, by way of example and with preference ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

Solvates in the context of the invention are described as those forms of the compounds of the invention which form a complex in the solid or liquid state by coordination with solvent molecules. Hydrates are a specific form of the solvates in which the coordination is with water. Solvates preferred in the context of the present invention are hydrates.

The compounds of the invention may, depending on their structure, exist in different stereoisomeric forms, i.e. in the form of configurational isomers or else, if appropriate, as conformational isomers (enantiomers and/or diastereomers, including those in the case of atropisomers). The present invention therefore encompasses the enantiomers and diastereomers, and the respective mixtures thereof. The stereoisomerically homogeneous constituents can be isolated from such mixtures of enantiomers and/or diastereomers in a known manner; chromatographic processes are preferably used for this purpose, especially HPLC chromatography on an achiral or chiral phase.

If the compounds of the invention can occur in tautomeric forms, the present invention encompasses all the tautomeric forms.

The present invention also encompasses all suitable isotopic variants of the compounds of the invention. An isotopic variant of a compound of the invention is understood here to mean a compound in which at least one atom within the compound of the invention has been exchanged for another atom of the same atomic number, but with a different atomic mass from the atomic mass which usually or predominantly occurs in nature. Examples of isotopes which can be incorporated into a compound of the invention are those of hydrogen, carbon, nitrogen, oxygen, phosphorus, sulfur, fluorine, chlorine, bromine and iodine, such as ²H (deuterium), ³H (tritium), ¹³C, ¹⁴C, ¹⁵N, ¹⁷O, ¹⁸O, ³²P, ³³P, ³³S, ³⁴S, ³⁵, ³⁶S, ¹⁸F, ³⁶Cl, ⁸²Br, ¹²³I, ¹²⁴I, ¹²⁹I and ¹³¹I. Particular isotopic variants of a compound of the invention, especially those in which one or more radioactive isotopes have been incorporated, may be beneficial, for example, for the examination of the mechanism of action or of the active compound distribution in the body; due to the comparatively easy preparability and detectability, especially compounds labeled with ³H or ¹⁴C isotopes are suitable for this purpose. In addition, the incorporation of isotopes, for example of deuterium, may lead to particular therapeutic benefits as a consequence of greater metabolic stability of the compound, for example an extension of the half-life in the body or a reduction in the active dose required; such modifications of the compounds according to the invention may therefore in some cases also constitute a preferred embodiment of the present invention. Isotopic variants of the compounds of the invention can be prepared by the processes known to those skilled in the art, for example by the methods described further down and the procedures described in the working examples, by using corresponding isotopic modifications of the respective reagents and/or starting materials.

The present invention additionally also encompasses prodrugs of the compounds of the invention. The term “prodrugs” in this context refers to compounds which may themselves be biologically active or inactive but are reacted (for example metabolically or hydrolytically) to give compounds according to the invention during their residence time in the body.

In the context of the present invention, unless specified otherwise, the substituents are defined as follows:

Alkyl in the context of the invention is a straight-chain or branched alkyl radical having the particular number of carbon atoms specified. By way of example and with preference, mention may be made of the following: methyl, ethyl, n-propyl, isopropyl, n-butyl, isobutyl, 1-methylpropyl, tert-butyl, n-pentyl, isopentyl, 1-ethylpropyl, 1-methylbutyl, 2-methylbutyl, 3-methylbutyl, n-hexyl, 1-methylpentyl, 2-methylpentyl, 3-methylpentyl, 4-methylpentyl, 3,3-dimethylbutyl, 1-ethylbutyl, 2-ethylbutyl.

Cycloalkyl or carbocycle or carbocyclyl in the context of the invention is a monocyclic, bicyclic or tricyclic saturated alkyl radical having the particular number of carbon atoms specified. By way of example and with preference, mention may be made of the following: cyclopropyl, cyclobutyl, cyclopentyl, cyclohexyl, cycloheptyl and adamantyl.

Alkenyl in the context of the invention is a straight-chain or branched alkenyl radical having 2 to 6 carbon atoms and one or two double bonds. Preference is given to a straight-chain or branched alkenyl radical having 2 to 4 carbon atoms and one double bond. By way of example and with preference, mention may be made of the following: vinyl, allyl, isopropenyl and n-but-2-en-1-yl.

Alkenyl in the context of the invention is a straight-chain or branched alkynyl radical having 2 to 6 carbon atoms and one triple bond. By way of example and with preference, mention may be made of the following: ethynyl, n-prop-1-yn-1-yl, n-prop-2-yn-1-yl, n-but-2-yn-1-yl and n-but-3-yn-1-yl.

Alkanediyl in the context of the invention is a straight-chain or branched divalent alkyl radical having 1 to 4 carbon atoms. By way of example and with preference, mention may be made of the following: methylene, 1,2-ethylene, ethane-1,1-diyl, 1,3-propylene, propane-1,1-diyl, propane-1,2-diyl, propane-2,2-diyl, 1,4-butylene, butane-1,2-diyl, butane-1,3-diyl and butane-2,3-diyl.

Monoalkylamino in the context of the invention is an amino group having a straight-chain or branched alkyl substituent having 1 to 4 carbon atoms. By way of example and with preference, mention may be made of the following: methylamino, ethylamino, n-propylamino, isopropylamino and tert-butylamino.

Dialkylamino in the context of the invention is an amino group having two identical or different straight-chain or branched alkyl substituents each having 1 to 4 carbon atoms. By way of example and with preference, mention may be made of the following: N,N-dimethylamino, N,N-diethylamino, N-ethyl-N-methylamino, N-methyl-N-n-propylamino, N-isopropyl-N-n-propylamino and N-tert-butyl-N-methylamino.

Alkoxy in the context of the invention is a straight-chain or branched alkoxy radical having 1 to 4 carbon atoms. By way of example and with preference, mention may be made of the following: methoxy, ethoxy, n-propoxy, isopropoxy, 1-methylpropoxy, n-butoxy, isobutoxy and tert-butoxy.

Alkoxycarbonyl in the context of the invention is a straight-chain or branched alkoxy radical having 1 to 4 carbon atoms and a carbonyl group attached to the oxygen atom. By way of example and with preference, mention may be made of the following: methoxycarbonyl, ethoxycarbonyl, n-propoxycarbonyl, isopropoxycarbonyl and tert-butoxycarbonyl.

Alkylsulfonyl in the context of the invention is a straight-chain or branched alkyl radical which has 1 to 4 carbon atoms and is attached via a sulfonyl group. The following may be mentioned by way of example and by way of preference: methylsulfonyl, ethylsulfonyl, n-propylsulfonyl, isopropylsulfonyl, n-butylsulfonyl and tert-butylsulfonyl.

A 4- to 7-membered or 5- to 10-membered heterocyclyl in the context of the invention is a monocyclic saturated heterocycle which has a total of 4 to 7 ring atoms or 5 to 10 ring atoms, contains one or two ring heteroatoms from the group consisting of N, O, S, SO and SO₂ and is attached via a ring carbon atom or, where appropriate, a ring nitrogen atom. The following may be mentioned by way of example: azetidinyl, oxetanyl, pynolidinyl, pyrazolidinyl, tetrahydrofuranyl, thiolanyl, piperidinyl, piperazinyl, tetrahydropyranyl, tetrahydrothiopyranyl, morpholinyl, thiomorpholinyl, hexahydroazepinyl and hexahydro-1,4-diazepinyl. Preference is given to azetidinyl, oxetanyl, pyrrolidinyl, tetrahydrofuranyl, piperidinyl, piperazinyl, tetrahydropyranyl and morpholinyl.

A 4- to 7-membered azaheterocycle in the context of the invention, in R¹¹ and R¹², is a monocyclic saturated heterocycle which has a total of 4 to 7 ring atoms, contains a nitrogen atom and may additionally contain a further ring heteroatom from the group consisting of N, O, S, SO and SO₂, and is attached via a ring nitrogen atom. The following may be mentioned by way of example: azetidinyl, pyrrolidinyl, pyrazolidinyl, piperidinyl, piperazinyl, morpholinyl, thiomorpholinyl, 1,1-dioxothiomorpholinyl, hexahydroazepinyl and hexahydro-1,4-diazepinyl.

5- to 10-membered azaheterocyclyl in the context of the invention, in R¹⁵, is a monocyclic or bicyclic, saturated or partly unsaturated heterocycle which has a total of 5 to 10 ring atoms, contains a nitrogen atom and may additionally contain one or two further ring heteroatom(s) from the group consisting of N, O, S, SO and SO₂, and is attached via a ring carbon atom. The following may be mentioned by way of example: pyrrolidinyl, pyrazolidinyl, piperidinyl, piperazinyl, morpholinyl, thiomorpholinyl, 1,1-dioxothiomorpholinyl, hexahydroazepinyl, hexahydro-1,4-diazepinyl, 1,2,3,4-tetrahydroisoquinolinyl, 1,2,3,4-tetrahydroquinolinyl, indolinyl, 8-azabicyclo[3.2.1]octanyl, 9-azabicyclo[3.3.1]nonanyl, 3-azabicyclo[4.1.0]heptanyl and quinuclidinyl.

Heteroaryl in the context of the invention is a mono- or bicyclic aromatic heterocycle (heteroaromatic) which contains up to four identical or different ring heteroatoms from the group consisting of N, O and S and is attached via a ring carbon atom or, where appropriate, via a ring nitrogen atom. By way of example and with preference, mention may be made of the following: furyl, pyrrolyl, thienyl, pyrazolyl, imidazolyl, quinolinyl, thiazolyl, oxazolyl, isoxazolyl, isothiazolyl, triazolyl, oxadiazolyl, thiadiazolyl, tetrazolyl, pyridyl, pyrimidinyl, pyridazinyl, pyrazinyl and triazinyl.

Halogen in the context of the invention includes fluorine, chlorine, bromine and iodine. Preference is given to chlorine or fluorine.

In the formula of the group that R³ or R¹ may represent, the end point of the line marked by the symbol * and # does not represent a carbon atom or a CH₂ group but is part of the bond to the respective atom to which R³ or R¹ is attached.

When radicals in the compounds of the invention are substituted, the radicals may be mono- or polysubstituted, unless specified otherwise. In the context of the present invention, all radicals which occur more than once are defined independently of one another. Substitution by one, two or three identical or different substituents is preferred.

In the context of the present invention, the term “treatment” or “treating” includes inhibition, retardation, checking, alleviating, attenuating, restricting, reducing, suppressing, repelling or healing of a disease, a condition, a disorder, an injury or a health problem, or the development, the course or the progression of such states and/or the symptoms of such states. The term “therapy” is understood here to be synonymous with the term “treatment”.

The terms “prevention”, “prophylaxis” and “preclusion” are used synonymously in the context of the present invention and refer to the avoidance or reduction of the risk of contracting, experiencing, suffering from or having a disease, a condition, a disorder, an injury or a health problem, or a development or advancement of such states and/or the symptoms of such states.

The treatment or prevention of a disease, a condition, a disorder, an injury or a health problem may be partial or complete.

In the context of the present invention, preference is given to compounds of the formulae (I-A) and (I-B) in which
A represents CH₂, CD₂ or CH(CH₃),
R¹ represents (C₃-C₇)-cycloalkyl, pyridyl or phenyl,
   where pyridyl is substituted by 1 or 2 fluorine substituents,
   and
   where phenyl may be substituted by 1 to 4 substituents independently of one another selected from the group consisting of halogen, cyano, monofluoromethyl, difluoromethyl, trifluoromethyl, (C₁-C₄)-alkyl, (C₁-C₄)-alkoxy and (C₃-C₅)-cyclopropyl,
R² represents hydrogen, (C₁-C₄)-alkyl, cyclopropyl, difluoromethyl or trifluoromethyl,
R³ represents hydrogen, (C₁-C₄)-alkyl, cyclopropyl, difluoromethyl or trifluoromethyl,
R⁴ represents hydrogen or (C₁-C₄)-alkyl,
R⁵ represents a group of the formula

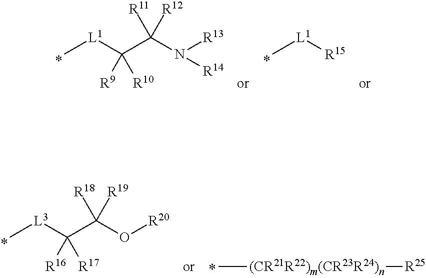

where
* represents the point of attachment to the amino group,
L¹ represents a bond, methanediyl or 1,2-ethanediyl,
L² represents a bond, methanediyl or 1,2-ethanediyl,
L³ represents a bond, methanediyl or 1,2-ethanediyl,
R⁹ represents hydrogen, (C₁-C₆)-alkyl, (C₃-C₇)-cycloalkyl, 5- or 6-membered heteroaryl or phenyl,
   in which (C₁-C₆)-alkyl may be substituted by 1 to 5 fluorine substituents,
   and
   in which phenyl and 5- or 6-membered heteroaryl may be substituted by 1 to 3 substituents independently of one another selected from the group consisting of fluorine, chlorine, bromine, cyano, trifluoromethyl, methyl, ethyl, methoxy or ethoxy,
R¹⁰ represents hydrogen or (C₁-C₄)-alkyl,
or
R⁹ and R¹⁰ together with the carbon atom to which they are attached form a 3- to 5-membered carbocycle,
R¹¹ represents hydrogen, (C₁-C₈)-alkyl, (C₃-C₅)-cycloalkyl, 5- or 6-membered heteroaryl or phenyl,
   in which (C₁-C₈)-alkyl may be substituted by 1 to 5 fluorine substituents,
   in which (C₁-C₈)-alkyl may be substituted by (C₁-C₄)-alkoxy, benzyloxy, phenoxy or phenyl,
      in which benzyloxy, phenoxy and phenyl may be substituted by 1 to 3 substituents independently of one another selected from the group consisting of fluorine, chlorine, bromine, methoxy and ethoxy,
   in which (C₃-C₅)-cycloalkyl may be substituted by 1 or 2 fluorine or (C₁-C₄)-alkyl substituents,
   and
   in which phenyl and 5- or 6-membered heteroaryl may be substituted by 1 to 3 substituents independently of one another selected from the group consisting of fluorine, chlorine, bromine, cyano, trifluoromethyl, methyl, ethyl, methoxy or ethoxy,
R¹² represents hydrogen or (C₁-C₄)-alkyl,
or
R¹¹ and R¹² together with the carbon atom to which they are attached form a 3- to 5-membered carbocycle,
or
R⁹ and R¹¹ together with the carbon atoms to which they are attached form a 3- to 6-membered carbocycle or a 4- to 7-membered heterocycle,
with the proviso that not more than one of the R⁹ and R¹⁰, R¹¹ and R¹², and R⁹ and R¹¹ radical pairs at the same time forms a carbocycle,
and
with the proviso that the R⁹ and R¹¹ radicals are not both simultaneously phenyl or 5- or 6-membered heteroaryl,
R¹³ represents hydrogen or (C₁-C₄)-alkyl,
   in which (C₁-C₄)-alkyl may be substituted by 1 to 5 fluorine substituents,
R¹⁴ represents hydrogen, (C₁-C₆)-alkyl or (C₃-C₇)-cycloalkyl,
   in which (C₁-C₆)-alkyl may be substituted by 1 to 5 fluorine substituents,
or
R¹³ and R¹⁴ together with the nitrogen atom to which they are attached form a 4- to 7-membered azaheterocycle,
R¹⁵ represents 5- to 10-membered azaheterocyclyl attached via a ring carbon atom,
   in which 5- to 1 0-membered azaheterocyclyl attached via a ring carbon atom may be substituted by 1 to 5 substituents independently of one another selected from the group consisting of fluorine, methyl and ethyl,
R¹⁶ represents hydrogen, (C₁-C₁₀)-alkyl, (C₃-C₅)-cycloalkyl, —(C═O)NR²⁶R²⁷, 5- or 6-membered heteroaryl or phenyl,
   in which (C₁-C₁₀)-alkyl may be substituted by difluoromethoxy, trifluoromethoxy, hydroxy or (C₁-C₄)-alkoxy and up to hexasubstituted by fluorine,
      in which R²⁶ represents hydrogen, (C₁-C₄)-alkyl, phenyl or naphthyl,
      in which R²⁷ represents hydrogen,
   and
   in which phenyl and 5- or 6-membered heteroaryl may be substituted by 1 to 3 substituents independently of one another selected from the group consisting of fluorine, chlorine, bromine, trifluoromethyl, methyl and ethyl,
R¹⁷ represents hydrogen or (C₁-C₄)-alkyl,
or
R¹⁶ and R⁷ together with the carbon atom to which they are attached form a 3- to 5-membered carbocycle,
R¹⁸ represents hydrogen, (C₁-C₆)-alkyl or (C₃-C₅)-cycloalkyl,
   in which (C₁-C₆)-alkyl may be substituted by 1 to 5 fluorine substituents,
   and
   in which (C₃-Cₛ)-cycloalkyl may be substituted by 1 or 2 substituents independently of one another selected from the group consisting of fluorine, trifluoromethyl, hydroxy and (C₁-C₄)-alkyl,
R¹⁹ represents hydrogen or (C₁-C₄)-alkyl,
or
R¹⁸ and R¹⁹ together with the carbon atom to which they are attached form a 3- to 6-membered carbocycle,
   in which the 3- to 6-membered carbocycle may be substituted by 1 or 2 fluorine or (C₁-C₄)-alkyl substituents,
or
R¹⁶ and R¹⁸ together with the carbon atoms to which they are attached form a 3- to 6-membered carbocycle or a 4- to 7-membered heterocycle,
with the proviso that not more than one of the R¹⁶ and R¹⁷, R¹⁸ and R¹⁹, and R¹⁶ and R¹⁸ radical pairs at the same time forms a carbo- or heterocycle,
R²⁰ represents hydrogen or (C₁-C₄)-alkyl,
   in which (C₁-C₄)-alkyl may be substituted by 1 to 5 fluorine substituents,
m represents 0 or 1,
n represents 0 or 1,
R²¹ represents hydrogen, cyano or (C₁-C₄)-alkyl,
   in which (C₁-C₄)-alkyl may be substituted by 1 to 5 fluorine substituents,
R²² represents hydrogen or (C₁-C₄)-alkyl,
   in which (C₁-C₄)-alkyl may be substituted by 1 to 5 fluorine substituents,
R²³ represents hydrogen or (C₁-C₆)-alkyl,
   in which (C₁-C₆)-alkyl may be substituted by 1 to 5 fluorine substituents,
R²⁴ represents hydrogen or (C₁-C₄)-alkyl,
   in which (C₁-C₄)-alkyl may be substituted by 1 to 5 fluorine substituents,
or
R²¹ and R²² together with the carbon atom to which they are attached form a 3- to 5-membered carbocycle,
   in which the 3- to 5-membered carbocycle may be substituted by 1 or 2 substituents independently of one another selected from the group consisting of fluorine, methyl and ethyl,
or
R²³ and R²⁴ together with the carbon atom to which they are attached form a 3- to 5-membered carbocycle,
   in which the 3- to 5-membered carbocycle may be substituted by 1 or 2 substituents independently of one another selected from the group consisting of fluorine, methyl and ethyl,
or
R²¹ and R²³ together with the carbon atom to which they are attached form a 3- to 5-membered carbocycle,
   in which the 3- to 5-membered carbocycle may be substituted by 1 or 2 substituents independently of one another selected from the group consisting of fluorine, methyl and ethyl,
with the proviso that not more than one of the R²¹ and R²², R²³ and R²⁴, and R²¹ and R²³ radical pairs at the same time forms a carbocycle,
R²⁵ represents (C₁-C₆)-alkyl, 5- or 6-membered heterocyclyl attached via a ring carbon atom, 5- or 6-membered carbocyclyl, phenyl or 5- to 10-membered heteroaryl,
   where (C₁-C₆)-alkyl may be substituted by cyano or up to three times by fluorine,
   in which phenyl may be substituted by 1 to 3 substituents independently of one another selected from the group consisting of halogen, cyano, trifluoromethyl, difluoromethyl, (C₁-C₄)-alkyl, (C₁-C₄)-alkoxy and hydroxy,
   in which phenyl may be substituted by 5- or 6-membered heteroaryl,
   in which 5- or 6-membered heteroaryl may be substituted by 1 to 3 substituents independently of one another selected from the group consisting of fluorine, chlorine, cyano, trifluoromethyl, (C₁-C₄)-alkoxy and amino,
      in which (C₁-C₄)-alkyl may be substituted by 1 to 3 substituents independently of one another selected from the group consisting of fluorine, chlorine, bromine, cyano, hydroxy, amino, trifluoromethyl, difluoromethyl, (C₁-C₄)-alkoxy, trifluoromethoxy, difluoromethoxy and phenyl,
         in which phenyl may be substituted by 1 to 3 substituents independently of one another selected from the group consisting of fluorine, chlorine, bromine, (C₁-C₄)-alkyl and (C₁-C₄)-alkoxy,
   in which 5- or 6-membered heterocyclyl attached via a ring carbon atom may be substituted by 1 to 3 substituents independently of one another selected from the group consisting of oxo, fluorine, trifluoromethyl, hydroxy and (C₁-C₄)-alkyl,
   in which 5- or 6-membered heterocyclyl attached via a ring carbon atom may be fused to a phenyl ring or a pyridyl ring, which for their part may be substituted by 1 to 3 substituents selected from the group consisting of fluorine, chlorine, bromine, (C₁-C₄)-alkyl and trifluoromethyl,
   and
   in which 5- or 6-membered carbocyclyl may be substituted by 1 or 2 substituents independently of one another selected from the group consisting of trifluoromethyl, fluorine, cyano, hydroxy, amino and methyl,
   in which 5- or 6-membered carbocyclyl may be fused to a phenyl ring or a pyridyl ring, which for their part may be substituted by 1 to 3 substituents selected from the group consisting of fluorine, chlorine, bromine, methyl, ethyl, methoxy, ethoxy and trifluoromethyl,
R⁶ represents hydrogen,
R⁷ represents hydrogen, fluorine, chlorine, bromine, cyano, difluoromethyl, trifluoromethyl, (C₁-C₄)-alkyl, (C₂-C₄)-alkynyl or (C₃-C₅)-cycloalkyl,
R⁸ represents hydrogen or fluorine,

and the N-oxides, salts, solvates, salts of the N-oxides and solvates of the N-oxides and salts thereof.

In the context of the present invention, preference is given to compounds of the formula (I-A) in which
A represents CH₂,
R¹ represents phenyl,
   where phenyl may be substituted by 1 to 4 substituents independently of one another selected from the group consisting of fluorine and chlorine,
R² represents hydrogen or methyl,
R³ represents hydrogen or methyl,
R⁴ represents hydrogen,
R⁵ represents a group of the formula

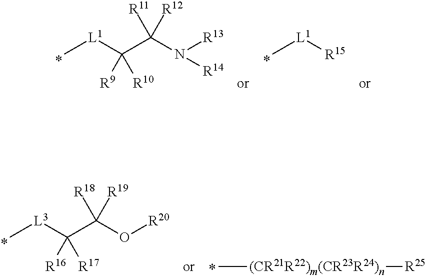

where
* represents the point of attachment to the amino group,
L¹ represents a bond or methanediyl,
L²represents a bond,
L³represents a bond, methanediyl or 1,2-ethanediyl,
R⁹ represents hydrogen,
R¹⁰ represents hydrogen,
R¹¹ represents hydrogen, (C₁-C₈)-alkyl, cyclopropyl or cyclobutyl,
   in which (C₁-C₈)-alkyl may be substituted by phenyl,
      in which phenyl may be substituted by 1 or 2 substituents independently of one another selected from the group consisting of fluorine, chlorine and methoxy,
   in which (C₁-C₈)-alkyl may be substituted by 1 to 5 fluorine substituents,
R¹² represents hydrogen or (C₁-C₄)-alkyl,
or
R¹¹ and R¹² together with the carbon atom to which they are attached form a 3- to 5-membered carbocycle,
R¹³ represents hydrogen, methyl or ethyl,
   in which ethyl may be substituted by 1 to 3 fluorine substituents,
R¹⁴ represents hydrogen, methyl or cyclopropyl,
or
R¹³ and R¹⁴ together with the nitrogen atom to which they are attached form a morpholinyl ring or piperidinyl ring,
R¹⁵ represents 9-azabicyclo[3.3.1]nonan-3-yl or piperidin-4-yl,
   in which 9-azabicyclo[3.3.1]nonan-3-yl is substituted by methyl,
   in which piperidin-4-yl is substituted by 1 to 5 methyl substituents,
R¹⁶ represents hydrogen, (C₁-C₈)-alkyl, —(C═O)NR²⁶R²⁷ or phenyl,
   in which (C₁-C₈)-alkyl may be substituted by a hydroxy or methoxy radical or up to five times by fluorine,
   in which R²⁶ represents phenyl or naphthyl,
   in which R²⁷ represents hydrogen,
   and
   in which phenyl may be substituted by 1 to 3 substituents independently of one another selected from the group consisting of fluorine, chlorine and methyl,
R¹⁷ represents hydrogen or (C₁-C₄)-alkyl,
R¹⁸ represents hydrogen, (C₁-C₆)-alkyl or cyclopropyl,
   in which (C₁-C₆)-alkyl may be substituted by 1 to 5 fluorine substituents,
R¹⁹ represents hydrogen or (C₁-C₄)-alkyl,
or
R¹⁸ and R¹⁹ together with the carbon atom to which they are attached form a 3- to 5-membered carbocycle,
R²⁰ represents hydrogen or (C₁-C₄)-alkyl,
   in which (C₁-C₄)-alkyl may be substituted by 1 to 5 fluorine substituents,
m represents 0 or 1,
n represents 0 or 1,
R²¹ represents hydrogen, cyano or methyl,
   in which methyl may be substituted by 1 to 3 fluorine substituents,
R²² represents hydrogen or methyl,
   in which methyl may be substituted by 1 to 3 fluorine substituents,
R²³ represents hydrogen or methyl,
   in which methyl may be substituted by 1 to 3 fluorine substituents,
R²⁴ represents hydrogen or methyl,
   in which methyl may be substituted by 1 to 3 fluorine substituents,
or
R²¹ and R²² together with the carbon atom to which they are attached form a 3- to 5-membered carbocycle,
or
R²¹ and R²³ together with the carbon atom to which they are attached form a 3- to 5-membered carbocycle,
with the proviso that not more than one of the R²¹ and R²², and R²¹ and R²³ radical pairs at the same time forms a carbocycle,
R²⁵ represents (C₁-C₆)-alkyl, 2-oxopyrrolidin-3-yl, 2-oxotetrahydrofuran-3-yl, cyclopropyl, cyclobutyl, cyclopentyl, cyclohexyl, phenyl, 2,3-dihydro-1H-inden-1-yl, 3,4-dihydro-2H-pyrano[2,3-b]pyridin-4-yl, 1,2,3,4-tetrahydrochinolin-4-yl, 1,2,4-oxadiazol-5-yl, 1H-imidazol-2-yl, 1H-pyrazol-4-yl, pyridin-3-yl, pyrimidin-5-yl, quinolin-4-yl or pyrazolo[1,5-a]pyridin-3-yl,
   in which (C₁-C₆)-alkyl may be up to trisubstituted by fluorine,
   in which phenyl may be substituted by 1 or 2 substituents independently of one another selected from the group consisting of fluorine, chlorine, cyano, trifluoromethyl, methyl, ethyl and methoxy,
   in which phenyl may be substituted by pyridyl or 1H-imidazol-1-yl,
   in which 1,2,4-oxadiazol-5-yl, 1H-imidazol-2-yl, 1H-pyrazol-4-yl, pyridin-3-yl, pyrimidin-5-yl, 2,3-dihydro-1H-inden-1-yl, quinolin-4-yl or pyrazolo[1,5-a]pyridin-3-yl may be substituted by 1 to 3 substituents independently of one another selected from the group consisting of fluorine, chlorine, trifluoromethyl, (C₁-C₃)-alkyl, amino and hydroxyl,
      in which (C₁-C₃)-alkyl may be substituted by fluorine, hydroxy, amino, phenyl or trifluoromethyl,
         in which phenyl may be substituted by 1 or 2 substituents independently selected from the group consisting of fluorine and chlorine,
   in which cyclopropyl, cyclobutyl, cyclopentyl and cyclohexyl are substituted by hydroxy,
R⁶ represents hydrogen,
R⁷ represents hydrogen or methyl,
R⁸ represents hydrogen,

and the N-oxides, salts, solvates, salts of the N-oxides and solvates of the N-oxides and salts thereof.

In the context of the present invention, preference is given to compounds of the formula (I-A) in which
A represents CH₂,
R¹ represents phenyl,
   where phenyl may be substituted by 1 to 3 fluorine substituents,
R² represents hydrogen or methyl,
R³ represents hydrogen,
R⁴ represents hydrogen,
R⁵ represents a group of the formula

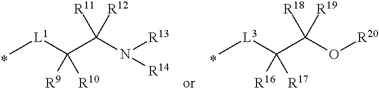

where
* represents the point of attachment to the amino group,
L¹ represents a bond,
L³ represents a bond, methanediyl or 1,2-ethanediyl,
R⁹ represents hydrogen,
R¹⁰ represents hydrogen,
R¹¹ represents hydrogen, (C₁-C₈)-alkyl or cyclopropyl,
   in which (C₁-C₈)-alkyl may be substituted by phenyl,
      in which phenyl may be substituted by 1 or 2 substituents independently of one another selected from the group consisting of chlorine and methoxy,
   in which (C₁-C₈)-alkyl may be substituted by 1 to 5 fluorine substituents,
R¹² represents hydrogen or (C₁-C₄)-alkyl,
or
R¹¹ and R¹² together with the carbon atom to which they are attached form a 3- to 5-membered carbocycle,
R¹³ represents hydrogen, methyl or ethyl,
   in which ethyl may be substituted by 1 to 3 fluorine substituents,
R¹⁴ represents hydrogen, methyl or cyclopropyl,
or
R¹³ and R¹⁴ together with the nitrogen atom to which they are attached form a morpholinyl ring or piperidinyl ring,
R¹⁵ represents 9-azabicyclo[3.3.1]nonan-3-yl or piperidin-4-yl,
   in which 9-azabicyclo[3.3.1]nonan-3-yl is substituted by methyl,
   in which piperidin-4-yl is substituted by 1 to 5 methyl substituents,
R¹⁶ represents hydrogen, (C₁-C₈)-alkyl, —(C═O)NR²⁶R²⁷ or phenyl,
   in which (C₁-C₈)-alkyl may be substituted by a hydroxy or methoxy radical or up to five times by fluorine,
   in which R²⁶ represents phenyl or naphthyl,
   in which R²⁷ represents hydrogen,
   and
   in which phenyl may be substituted by 1 to 3 substituents independently of one another selected from the group consisting of fluorine, chlorine and methyl,
R¹⁷ represents hydrogen or (C₁-C₄)-alkyl,
R¹⁸ represents hydrogen, (C₁-C₆)-alkyl or cyclopropyl,
R¹⁹ represents hydrogen or (C₁-C₄)-alkyl,
or
R¹⁸ and R¹⁹ together with the carbon atom to which they are attached form a 3- to 5-membered carbocycle,
R²⁰ represents hydrogen or (C₁-C₄)-alkyl,
   in which (C₁-C₄)-alkyl may be substituted by 1 to 5 fluorine substituents,
R⁶ represents hydrogen,
R⁷ represents hydrogen or methyl,
R⁸ represents hydrogen,

and the N-oxides, salts, solvates, salts of the N-oxides and solvates of the N-oxides and salts thereof.

Particular preference is given in the context of the present invention to compounds of the formula (I-A) in which
A represents CH₂,
R¹ represents a phenyl group of the formula

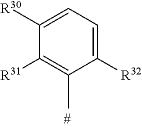

where
# represents the point of attachment to A,
and
R³⁰ represents hydrogen or fluorine,
R³¹ represents fluorine,
R³² represents fluorine,
R² represents hydrogen or methyl,
R³ represents hydrogen,
R⁴ represents hydrogen,
R⁵ represents a group of the formula

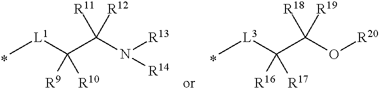

where
* represents the point of attachment to the amino group,
L¹ represents a bond,
L³ represents a bond,
R⁹ represents hydrogen,
R¹⁰ represents hydrogen,
R¹¹ represents hydrogen or (C₁-C₆)-alkyl,
   in which (C₁-C₆)-alkyl may be substituted by 1 to 5 fluorine substituents,
R¹² represents hydrogen, methyl or ethyl,
R¹³ represents hydrogen,
R¹⁴ represents hydrogen,
R¹⁶ represents hydrogen, (C₁-C₆)-alkyl, —(C═O)NR²⁶R²⁷ or phenyl,
   in which (C₁-C₆)-alkyl may be substituted by a hydroxy or methoxy radical or up to five times by fluorine,
   in which R²⁶ represents naphthyl,
   in which R²⁷ represents hydrogen,
   and
   in which phenyl may be substituted by fluorine,
R¹⁷ represents hydrogen, methyl or ethyl,
R¹⁸ represents hydrogen, methyl or ethyl,
R¹⁹ represents hydrogen, methyl or ethyl,
or
R¹⁸ and R¹⁹ together with the carbon atom to which they are attached form a cyclopropyl ring,
R²⁰ represents hydrogen,
R⁶ represents hydrogen,
R⁷ represents hydrogen or methyl,
R⁸ represents hydrogen,

and the N-oxides, salts, solvates, salts of the N-oxides and solvates of the N-oxides and salts thereof.

In the context of the present invention, preference is also given to compounds of the formula (I-A)

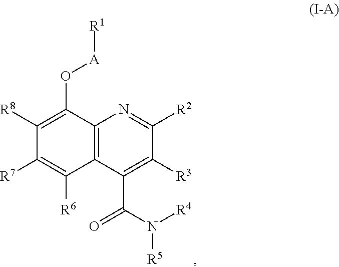

in which A, R¹, R², R³, R⁴, R⁵, R⁶, R⁷ and R⁸ have the meaning given above,

and the N-oxides, salts, solvates, salts of the N-oxides and solvates of the N-oxides and salts thereof.

In the context of the present invention, preference is also given to compounds of the formula (I-B)

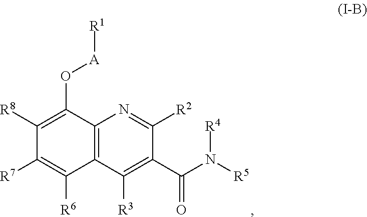

in which A, R¹, R², R³, R⁴, R⁵, R⁶, R⁷ and R⁸ have the meaning given above,

and the N-oxides, salts, solvates, salts of the N-oxides and solvates of the N-oxides and salts thereof.

In the context of the present invention, preference is also given to compounds of the formula (I-A) in which
R¹ represents a phenyl group of the formula

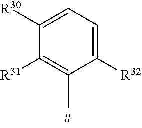

where
# represents the point of attachment to A,
and
R³⁰ represents hydrogen or fluorine,
R³¹ represents fluorine,
R³² represents fluorine,

and the N-oxides, salts, solvates, salts of the N-oxides and solvates of the N-oxides and salts thereof.

In the context of the present invention, preference is also given to compounds of the formula (I-A) in which
R² represents hydrogen,

and the N-oxides, salts, solvates, salts of the N-oxides and solvates of the N-oxides and salts thereof.

In the context of the present invention, preference is also given to compounds of the formula (I-A) in which
R² represents methyl,

and the N-oxides, salts, solvates, salts of the N-oxides and solvates of the N-oxides and salts thereof.

In the context of the present invention, preference is also given to compounds of the formula (I-A) in which
R³ represents hydrogen,

and the N-oxides, salts, solvates, salts of the N-oxides and solvates of the N-oxides and salts thereof.

In the context of the present invention, preference is also given to compounds of the formula (I-A) in which
R³ represents methyl,

and the N-oxides, salts, solvates, salts of the N-oxides and solvates of the N-oxides and salts thereof.

In the context of the present invention, preference is also given to compounds of the formula (I-A) in which
R⁴ represents hydrogen,

and the N-oxides, salts, solvates, salts of the N-oxides and solvates of the N-oxides and salts thereof.

In the context of the present invention, preference is also given to compounds of the formula (I-A) in which
R⁵ represents a group of the formula

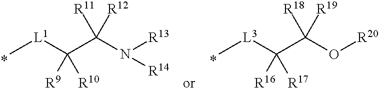

where
* represents the point of attachment to the amino group,
L¹ represents a bond,
L³represents a bond,
R⁹ represents hydrogen,
R¹⁰ represents hydrogen,
R¹¹ represents hydrogen, (C₁-C₆)-alkyl or cyclopropyl,
   in which (C₁-C₆)-alkyl may be substituted by 1 to 5 fluorine substituents,
R¹² represents hydrogen, methyl or ethyl,
R¹³ represents hydrogen,
R¹⁴ represents hydrogen,
R¹⁶ represents hydrogen, (C₁-C₆)-alkyl, —(C═O)NR²⁶R²⁷ or phenyl,
   in which (C₁-C₆)-alkyl may be substituted by a hydroxy or methoxy radical or up to five times by fluorine,
   in which R²⁶ represents naphthyl,
   in which R²⁷ represents hydrogen,
   and
   in which phenyl may be substituted by fluorine,
R¹⁷ represents hydrogen, methyl or ethyl,
R¹⁸ represents hydrogen or methyl,
R¹⁹ represents hydrogen or methyl,
or
R¹⁸ and R¹⁹ together with the carbon atom to which they are attached form a cyclopropyl ring,
R²⁰ represents hydrogen,

and the N-oxides, salts, solvates, salts of the N-oxides and solvates of the N-oxides and salts thereof.

In the context of the present invention, preference is also given to compounds of the formula (I-A) in which
R⁵ represents a group of the formula

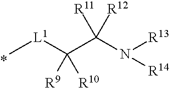

where
* represents the point of attachment to the amino group,
L¹ represents a bond,
R⁹ represents hydrogen,
R¹⁰ represents hydrogen,
R¹¹ represents hydrogen, (C₁-C₆)-alkyl or cyclopropyl,
   in which (C₁-C₆)-alkyl may be substituted by 1 to 5 fluorine substituents,
R¹²represents hydrogen, methyl or ethyl,
R¹³ represents hydrogen,
R¹⁴represents hydrogen,

and the N-oxides, salts, solvates, salts of the N-oxides and solvates of the N-oxides and salts thereof.

In the context of the present invention, preference is also given to compounds of the formula (I-A) in which
R⁵ represents a group of the formula

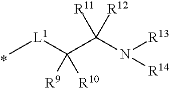

where
* represents the point of attachment to the amino group,
L¹ represents a bond,
R⁹ represents hydrogen,
R¹⁰ represents hydrogen,
R¹¹ represents hydrogen, (C₁-C₆)-alkyl or cyclopropyl,
   in which (C₁-C₆)-alkyl may be substituted by 1 to 5 fluorine substituents,
R¹² represents hydrogen,
R¹³ represents hydrogen,
R¹⁴ represents hydrogen,

and the N-oxides, salts, solvates, salts of the N-oxides and solvates of the N-oxides and salts thereof.

In the context of the present invention, preference is also given to compounds of the formula (I-A) in which
R⁵ represents a group of the formula

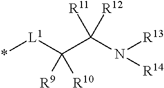

where
* represents the point of attachment to the amino group,
L¹ represents a bond,
R⁹ represents hydrogen,
R¹⁰ represents hydrogen,
R¹¹ represents (C₁-C₆)-alkyl,
   in which (C₁-C₆)-alkyl may be substituted by 1 to 5 fluorine substituents,
R¹² represents hydrogen,
R¹³ represents hydrogen,
R¹⁴ represents hydrogen,

and the N-oxides, salts, solvates, salts of the N-oxides and solvates of the N-oxides and salts thereof.

In the context of the present invention, preference is also given to compounds of the formula (I-A) in which
R⁵ represents a group of the formula

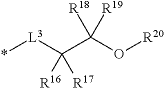

where
* represents the point of attachment to the amino group,
L³ represents a bond,
R¹⁶ represents hydrogen, (C₁-C₆)-alkyl, —(C═O)NR²⁶R²⁷ or phenyl,
   in which (C₁-C₆)-alkyl may be substituted by a hydroxy or methoxy radical or up to five times by fluorine,
   in which R²⁶ represents naphthyl,
   in which R²⁷ represents hydrogen,
   and
   in which phenyl may be substituted by fluorine,
R¹⁷ represents hydrogen, methyl or ethyl,
R¹⁸ represents hydrogen or methyl,
R¹⁹ represents hydrogen or methyl,
or
R¹⁸ and R¹⁹ together with the carbon atom to which they are attached form a cyclopropyl ring,
R²⁰ represents hydrogen,

and the N-oxides, salts, solvates, salts of the N-oxides and solvates of the N-oxides and salts thereof.

In the context of the present invention, preference is also given to compounds of the formula (I-A) in which
R⁵ represents a group of the formula

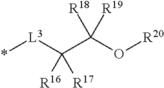

where
* represents the point of attachment to the amino group,
L³ represents a bond,
R¹⁶ represents hydrogen, (C₁-C₆)-alkyl, —(C═O)NR²⁶R²⁷or phenyl,
   in which (C₁-C₆)-alkyl may be substituted by a hydroxy or methoxy radical or up to five times by fluorine,
   in which R²⁶ represents naphthyl,
   in which R²⁷ represents hydrogen,
   and
   in which phenyl may be substituted by fluorine,
R¹⁷ represents hydrogen,
R¹⁸ represents hydrogen,
R¹⁹ represents hydrogen,
R²⁰ represents hydrogen,

and the N-oxides, salts, solvates, salts of the N-oxides and solvates of the N-oxides and salts thereof.

In the context of the present invention, preference is also given to compounds of the formula (I-A) in which
R⁵ is a group of the formula

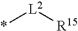

where
* represents the point of attachment to the amino group,
R¹⁵ represents 9-azabicyclo[3.3.1]nonan-3-yl or piperidin-4-yl,
   in which 9-azabicyclo[3.3.1]nonan-3-yl is substituted by methyl,
   in which piperidin-4-yl is substituted by 1 to 5 methyl substituents,

and the N-oxides, salts, solvates, salts of the N-oxides and solvates of the N-oxides and salts thereof.

In the context of the present invention, preference is also given to compounds of the formula (I-A) in which
R⁵ represents a group of the formula

*—(CR²¹R²²)ₘ(CR²³R²⁴)ₙ—R²⁵
where
* represents the point of attachment to the amino group,
R²⁵ represents (C₁-C₆)-alkyl, 2-oxopyrrolidin-3-yl, 2-oxotetrahydrofuran-3-yl, cyclopropyl, cyclobutyl, cyclopentyl, cyclohexyl, phenyl, 2,3-dihydro-1H-inden-1-yl, 3,4-dihydro-2H-pyrano[2,3-b]pyridin-4-yl, 1,2,3,4-tetrahydrochinolin-4-yl, 1,2,4-oxadiazol-5-yl, 1H-imidazol-2-yl, 1H-pyrazol-4-yl, pyridin-3-yl, pyrimidin-5-yl, quinolin-4-yl or pyrazolo[1,5-a]pyridin-3-yl,
   in which (C₁-C₆)-alkyl may be up to trisubstituted by fluorine,
   in which phenyl may be substituted by 1 or 2 substituents independently of one another selected from the group consisting of fluorine, chlorine, cyano, trifluoromethyl, methyl, ethyl and methoxy,
   in which phenyl may be substituted by pyridyl or 1H-imidazol-1-yl,
   in which 1,2,4-oxadiazol-5-yl, 1H-imidazol-2-yl, 1H-pyrazol-4-yl, pyridin-3-yl, pyrimidin-5-yl, 2,3-dihydro-1H-inden-1-yl, quino1in-4-yl or pyrazolo[1,5-a]pyridin-3-yl may be substituted by 1 to 3 substituents independently of one another selected from the group consisting of fluorine, chlorine, trifluoromethyl, (C₁-C₃)-alkyl, amino and hydroxyl,
      in which (C₁-C₃)-alkyl may be substituted by fluorine, hydroxy, amino, phenyl or trifluoromethyl,
         in which phenyl may be substituted by 1 or 2 substituents independently selected from the group consisting of fluorine and chlorine,
   where cyclopropyl, cyclobutyl, cyclopentyl and cyclohexyl are substituted by hydroxy,

and the N-oxides, salts, solvates, salts of the N-oxides and solvates of the N-oxides and salts thereof.

In the context of the present invention, preference is also given to compounds of the formula (I-A) in which
R⁷ represents hydrogen,

and the N-oxides, salts, solvates, salts of the N-oxides and solvates of the N-oxides and salts thereof.

In the context of the present invention, preference is also given to compounds of the formula (I-A) in which
R⁷ represents methyl,

and the N-oxides, salts, solvates, salts of the N-oxides and solvates of the N-oxides and salts thereof.

In the context of the present invention, preference is also given to compounds of the formula (I-A) in which
R⁸ represents hydrogen,

and the N-oxides, salts, solvates, salts of the N-oxides and solvates of the N-oxides and salts thereof.

The individual radical definitions specified in the respective combinations or preferred combinations of radicals are, independently of the respective combinations of the radicals specified, also replaced as desired by radical definitions of other combinations.

Particular preference is given to combinations of two or more of the preferred ranges mentioned above.

The invention further provides a process for preparing the compounds of the formulae (I-A) and (I-B) according to the invention, characterized in that

a compound of the formula (II-A) or (II-B)

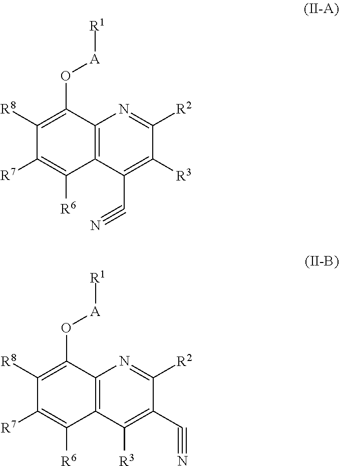

in which A, R¹, R², R³, R⁶, R⁷ and R⁸ each have the meanings given above,

is reacted in an inert solvent in the presence of a suitable base or acid to give a carboxylic acid of the formula (III-A) or (III-B)

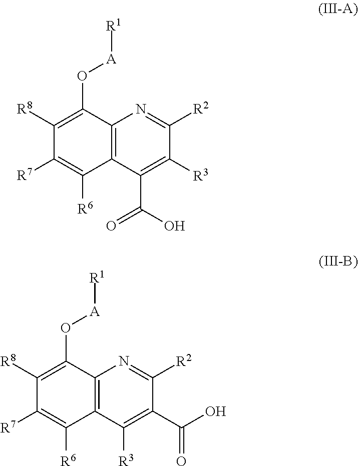

in which A, R¹, R², R³, R⁴, R⁶, R⁷ and R⁸ each have the meanings given above,

and this is subsequently reacted in an inert solvent under amide coupling conditions with an amine of the formula (IV)

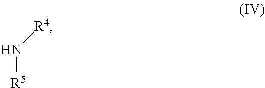

in which R⁴ and R⁵ each have the meanings given above,

then any protective groups present are detached, and the resulting compounds of the formula (I) are optionally converted with the appropriate (i) solvents and/or (ii) acids or bases to the solvates, salts and/or solvates of the salts thereof.

The preparation process described can be illustrated by way of example by the following synthesis scheme (Scheme 1):

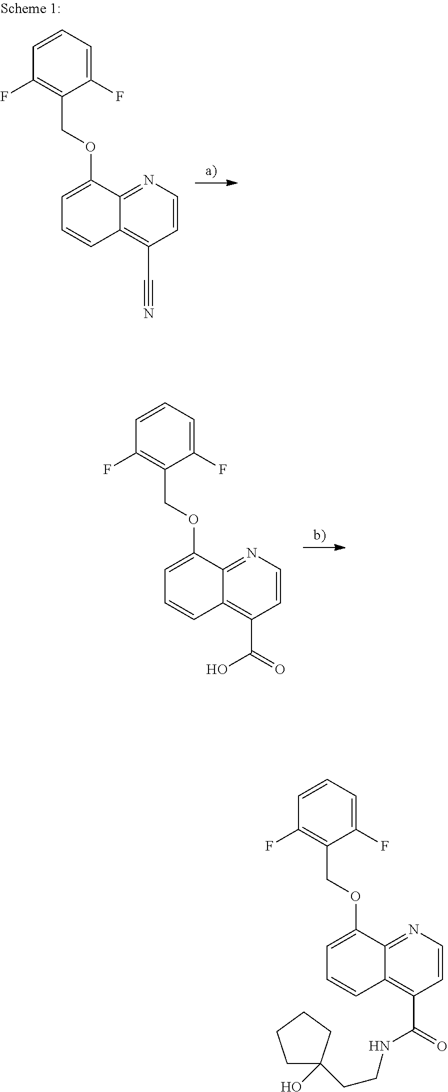 [SMILES: N#Cc1ccnc2c(OCc3c(F)cccc3F)cccc12 ; O=C(O)c1ccnc2c(OCc3c(F)cccc3F)cccc12 ; O=C(NCCC1(O)CCCC1)c1ccnc2c(OCc3c(F)cccc3F)cccc12]

[(a) sodium hydroxide, ethanol, reflux; (b) HATU, 4-methylmorpholine, 1-(2-aminoethyl)cyclopentanol, DMF, room temperature].

The compounds of the formula (IV) are commercially available, known from the literature or can be prepared in analogy to literature processes.

Inert solvents for the process step (III-A)+(IV)→(I-A) and (III-B)+(IV)→(I-B) are, for example, ethers such as diethyl ether, dioxane, tetrahydrofuran, glycol dimethyl ether or diethylene glycol dimethyl ether, hydrocarbons such as benzene, toluene, xylene, hexane, cyclohexane or mineral oil fractions, halohydrocarbons such as dichloromethane, trichloromethane, tetrachloromethane, 1,2-dichloroethane, trichloroethylene or chlorobenzene, or other solvents such as acetone, ethyl acetate, acetonitrile, pyridine, dimethyl sulfoxide, N,N-dimethylformamide, N,N-dimethylacetamide, N,N′-dimethylpropyleneurea (DMPU) or N-methylpyrrolidone (NMP). It is likewise possible to use mixtures of the solvents mentioned. Preference is given to dichloromethane, tetrahydrofuran, dimethylformamide or mixtures of these solvents.

Suitable for use as condensing agents for the amide formation in process steps (III-A)+(IV)→(I-A) and (III-B)+(IV)→(I-B) are, for example, carbodiimides such as N,N′-diethyl-, N,N′-dipropyl-, N,N′-diisopropyl-, N,N′-dicyclohexylcarbodiimide (DCC) or N-(3-dimethylaminopropyl)-N′-ethylcarbodiimide hydrochloride (EDC), phosgene derivatives such as N,N′-carbonyldiimidazole (CDI), 1,2-oxazolium compounds such as 2-ethyl-5-phenyl-1,2-oxazolium 3-sulfate or 2-tert-butyl-5-methylisoxazolium perchlorate, acylamino compounds such as 2-ethoxy-1-ethoxycarbonyl-1,2-dihydroquinoline or isobutyl chloroformate, propanephosphonic anhydride (T3P), 1-chloro-N,N,2-trimethylprop1-ene-1-amine, diethyl cyanophosphonate, bis(2-oxo-3-oxazolidinyl)phosphoryl chloride, benzotriazol-1-yloxytris(dimethylamino)phosphonium hexafluorophosphate, benzotriazol-1-yloxytris(pyrrolidino)phosphonium hexafluorophosphate (PyBOP), O-(benzotriazol-1-yl)-N,N,N,N′-tetramethyluronium tetrafluoroborate (TBTU), O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU), O-(7-azabenzotriazol-1-yl)-N,N,N′,N′-tetramethyluronium hexafluorophosphate (HATU) or O-(1H-6-chlorobenzotriazol-1-yl)-1,1,3,3-tetramethyluronium tetrafluoroborate (TCTU), optionally in combination with further auxiliaries such as 1-hydroxybenzotriazole (HOBt) or N-hydroxysuccinimide (HOSu), and also as bases alkali metal carbonates, for example sodium carbonate or potassium carbonate or sodium bicarbonate or potassium bicarbonate, or organic bases such as trialkylamines, for example triethylamine, N-methylmorpholine, N-methylpiperidine or N,N-diisopropylethylamine. Preference is given to using TBTU in combination with N-methylmorpholine, HATU in combination with N,N-diisopropylethylamine or 1-chloro-N,N,2-trimethylprop-1-en-1-amine

The condensation (III-A)+(IV)→(I-A) and (III-B)+(IV)→(I-B) is generally conducted within a temperature range from −20° C. to +100° C., preferably at 0° C. to +60° C. The conversion can be carried out under atmospheric, elevated or reduced pressure (for example from 0.5 to 5 bar). In general, the reaction is carried out at atmospheric pressure.

Alternatively, the carboxylic acid of the formula (III-A) or (III-B) can also first be converted to the corresponding carbonyl chloride and the latter can then be converted directly or in a separate reaction with an amine of the formula (IV) to the compounds of the invention. The formation of carbonyl chlorides from carboxylic acids is carried out by the methods known to those skilled in the art, for example by treatment with thionyl chloride, sulfuryl chloride or oxalyl chloride, in the presence of a suitable base, for example in the presence of pyridine, and optionally with addition of dimethylformamide, optionally in a suitable inert solvent.

The hydrolysis of the nitrile group of the compounds (II-A) and (II-B) to compounds of the formula (III-A) or (III-B) is carried out by treating the nitriles in inert solvents with suitable acids or bases.

Suitable acids for the hydrolysis of the nitrile group are, in general, sulfuric acid, hydrogen chloride/hydrochloric acid, hydrogen bromide/hydrobromic acid, phosphoric acid or acetic acid or mixtures thereof, optionally with addition of water. Preference is given to hydrogen chloride.

Suitable bases for the hydrolysis of the nitrile group are, in general, alkali metal or alkaline earth metal hydroxides such as, for example, sodium hydroxide, lithium hydroxide, potassium hydroxide or barium hydroxide or alkali metal or alkaline earth metal carbonates such as sodium carbonate, potassium carbonate or calcium carbonate. Particular preference is given to sodium hydroxide or lithium hydroxide.

Suitable inert solvents for these reactions are water, methanol, ethanol, isopropanol, butanol, pentanol, diethyl ether, tetrahydrofuran, dioxane or glycol dimethyl ether, or other solvents such as acetonitrile, dimethylformamide or dimethyl sulfoxide. It is also possible to use mixtures of the solvents mentioned. Preference is given to methanol and ethanol as mixtures with water.

The hydrolysis of the nitrile group generally takes place within a temperature range from 0□ to 180° C., preferably at +65° C. to 120° C.

These conversions can be performed at atmospheric, elevated or reduced pressure (for example from 0.5 to 5 bar). In general, the reaction is in each case carried out at atmospheric pressure.

The amino protective group used is preferably tert-butoxycarbonyl (Boc) or benzyloxycarbonyl (Cbz). The protecting group used for a hydroxy or carboxyl function is preferably tert-butyl or benzyl. These protective groups are detached by customary methods, preferably by reaction with a strong acid such as hydrogen chloride, hydrogen bromide or trifluoroacetic acid in an inert solvent such as dioxane, diethyl ether, dichloromethane or acetic acid; it is optionally also possible to effect the detachment without an additional inert solvent. In the case of benzyl and benzyloxycarbonyl as protective groups, these may also be removed by hydrogenolysis in the presence of a palladium catalyst. The detachment of the protective groups mentioned can optionally be undertaken simultaneously in a one-pot reaction or in separate reaction steps.

The compounds of the formulae (II-A) and (II-B) are known from the literature or can be prepared by

converting a compound of the formula (V-A) or (V-B)

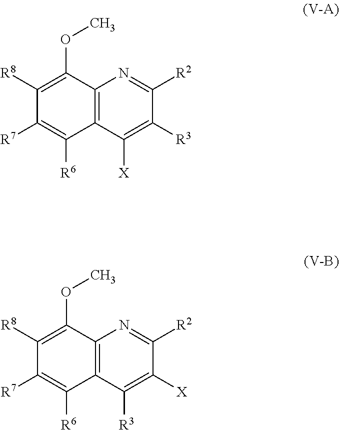

in which R², R³, R⁶, R⁷ and R⁸ have the meaning given above,

and
X represents a suitable leaving group, in particular chlorine, bromine or iodine,

by treatment with boron tribromide, boron trichloride, hydrogen chloride or hydrogen bromide into a compound (VI-A) or (VI-B)

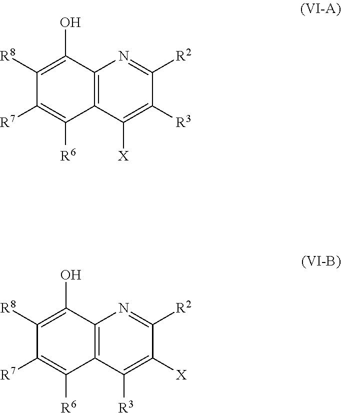

in which R², R³, R⁶, R⁷ and R⁸ have the meaning given above,

and
X represents a suitable leaving group, in particular chlorine, bromine or iodine,

and reacting this in an inert solvent in the presence of a suitable base with a compound of the formula (VII)

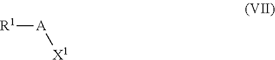

in which A and R¹ are each as defined above and
X¹ represents a suitable leaving group, in particular chlorine, bromine, iodine, mesylate, triflate or tosylate,

to give a compound of the formula (VIII-A) or (VIII-B)

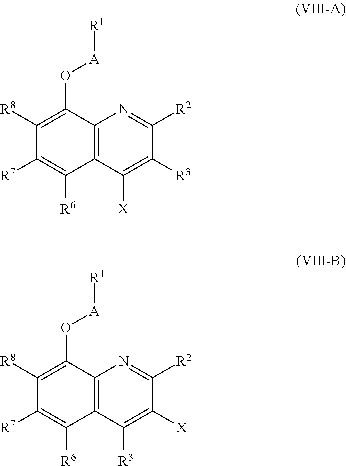

in which A, R¹, R², R³, R⁶, R⁷ and R⁸ each have the meanings given above,

and
X represents a suitable leaving group, in particular chlorine, bromine or iodine,

and then converting the latter in an inert solvent with copper(I) cyanide, zinc cyanide, sodium cyanide or potassium cyanide into a compound (II-A) or (II-B).

The process described is illustrated in an exemplary manner by the scheme (Scheme 2):

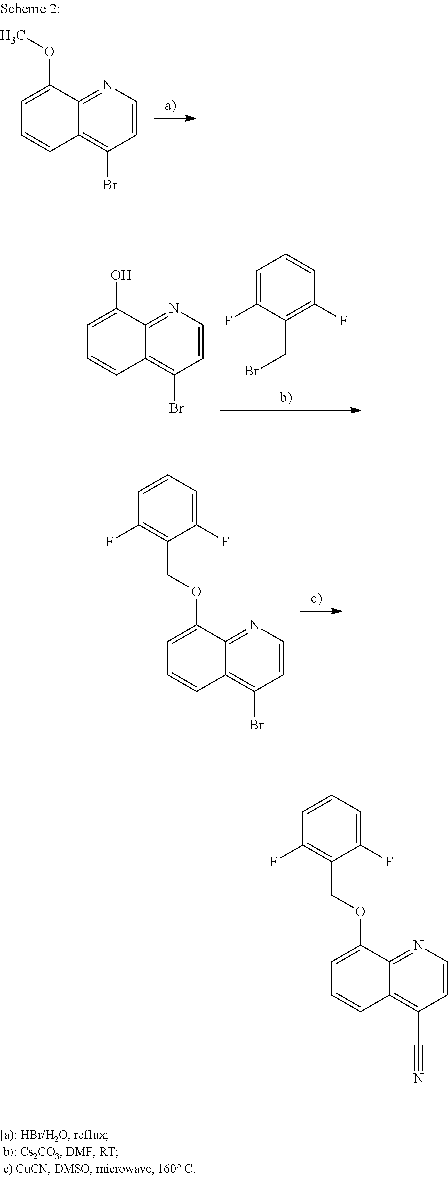

Here, removal of the methyl group in reaction step (V-A)→(VI-A) or (V-B)→(VI-B) takes place using customary methods known to the person skilled in the art, preferably by heating with a solution of boron tribromide in glacial acetic acid or water to from +100° C. to +130° C. or by treating with boron tribromide in dichloromethane at from −20° C. to +25° C.

The reaction can be carried out at atmospheric, elevated or reduced pressure (for example in the range from 0.5 to 5 bar); in general, the reactions are each carried out at atmospheric pressure.

Inert solvents for the process step (VI-A)+(VII)→(VIII-A) or (VI-B)+(VII)→(VIII-B) are, for example, ethers such as diethyl ether, dioxane, tetrahydrofuran, dimethoxymethane, glycol dimethyl ether or diethylene glycol dimethyl ether, or other solvents such as acetone, methyl ethyl ketone, ethyl acetate, acetonitrile, N,N-dimethylformamide (DMF), N,N-dimethylacetamide, dimethyl sulfoxide (DMSO), N,N′-dimethylpropyleneurea (DMPU), N-methylpyrrolidone (NMP). It is also possible to use mixtures of the solvents mentioned. Preference is given to using N,N-dimethylformamide (DMF).

Suitable bases for the process step (VI-A)+(VII)→(VIII-A) or (VI-B)+(VII)→(VIII-B) are the customary inorganic or organic bases. These preferably include alkali metal hydroxides, for example lithium hydroxide, sodium hydroxide or potassium hydroxide, alkali metal or alkaline earth metal carbonates such as lithium carbonate, sodium carbonate, potassium carbonate, calcium carbonate or cesium carbonate, optionally with addition of an alkali metal iodide, for example sodium iodide or potassium iodide, alkali metal alkoxides such as sodium methoxide or potassium methoxide, sodium ethoxide or potassium ethoxide or sodium or potassium tert-butoxide, alkali metal hydrides such as sodium hydride or potassium hydride, amides such as sodium amide, lithium bis(trimethylsilyl)amide or potassium bis(trimethylsilyl)amide or lithium diisopropylamide, or organic amines such as triethylamine, N-methylmorpholine, N-methylpiperidine, N,N-diisopropylethylamine, pyridine, 4-(N,N-dimethylamino)pyridine (DMAP), 1,5-diazabicyclo[4.3.0]non-5-ene (DBN), 1,8-diazabicyclo[5.4.0]undec-7-ene (DBU) or 1,4-diazabicyclo[2.2.2]octane) (DABCO®). Preference is given to using potassium carbonate, cesium carbonate, sodium tert-butoxide or potassium tert-butoxide.

The reaction is generally carried out within a temperature range from 0° C. to +120° C., preferably at +20° C. to +80° C., optionally in a microwave. The reaction can be carried out under atmospheric, elevated or reduced pressure (for example from 0.5 to 5 bar).

Here, exchange of the bromide group for the cyano group in reaction step (VIII-A)→(II-A) or (VIII-B)→(II-B) takes place using customary methods known to the person skilled in the art, preferably by reacting with copper(I) cyanide, zinc cyanide, sodium cyanide or potassium cyanide.

Also suitable are catalyzed reactions employing palladium catalysts [e.g. tetrakis(triphenylphosphine)palladium(0)].

Suitable solvents for the process step (VIII-A)→(II-A) or (VIII-B)→(II-B) are dioxane, tetrahydrofuran, glycol dimethyl ether or diethylene glycol dimethyl ether, acetone, acetonitrile, dimethyl sulfoxide (DMSO), N,N-dimethylformamide (DMF), N,N-dimethylacetamide, N-methylpyrrolidone (NMP) and water. It is likewise possible to use mixtures of the solvents mentioned. Preference is given to dimethyl sulfoxide and DMF.

The reaction is generally carried out within a temperature range from 25° C. to +200° C., preferably in a microwave.

The reaction can be carried out under atmospheric, elevated or reduced pressure (for example in the range from 0.5 to 5 bar).

The amino protecting group used is preferably tert-butoxycarbonyl (Boc) or benzyloxycarbonyl (Z). The protecting group used for a hydroxy or carboxyl function is preferably tent-butyl or benzyl. These protective groups are detached by customary methods, preferably by reaction with a strong acid such as hydrogen chloride, hydrogen bromide or trifluoroacetic acid in an inert solvent such as dioxane, diethyl ether, dichloromethane or acetic acid; it is optionally also possible to effect the detachment without an additional inert solvent. In the case of benzyl and benzyloxycarbonyl as protective groups, these may also be removed by hydrogenolysis in the presence of a palladium catalyst. The detachment of the protective groups mentioned can optionally be undertaken simultaneously in a one-pot reaction or in separate reaction steps.

Further compounds of the invention can optionally also be prepared by conversions of functional groups of individual substituents, especially those listed for R⁵, proceeding from the compounds of the formula (I-A) and (I-B) obtained by above processes. These conversions are performed by customary methods known to those skilled in the art and include, for example, reactions such as nucleophilic and electrophilic substitutions, oxidations, reductions, hydrogenations, transition metal-catalyzed coupling reactions, eliminations, alkylation, amination, esterification, ester hydrolysis, etherification, ether hydrolysis, formation of carbonamides, and introduction and removal of temporary protective groups.

The compounds of the invention have valuable pharmacological properties and can be used for prevention and treatment of diseases in humans and animals. The compounds of the invention offer a further treatment alternative and thus enlarge the field of pharmacy.

The compounds of the invention bring about vasorelaxation and inhibition of platelet aggregation, and lead to a decrease in blood pressure and to a rise in coronary blood flow. These effects are mediated by a direct stimulation of soluble guanylate cyclase and an intracellular rise in cGMP. In addition, the compounds of the invention enhance the action of substances which increase the cGMP level, for example EDRF (endothelium-derived relaxing factor), NO donors, protoporphyrin IX, arachidonic acid or phenylhydrazine derivatives.

The compounds of the invention are suitable for the treatment and/or prophylaxis of cardiovascular, pulmonary, thromboembolic and fibrotic disorders.

Accordingly, the compounds according to the invention can be used in medicaments for the treatment and/or prophylaxis of cardiovascular disorders such as, for example, high blood pressure (hypertension), resistant hypertension, acute and chronic heart failure, coronary heart disease, stable and unstable angina pectoris, peripheral and cardiac vascular disorders, arrhythmias, atrial and ventricular arrhythmias and impaired conduction such as, for example, atrioventricular blocks degrees I-III (AB block supraventricular tachyarrhythmia, atrial fibrillation, atrial flutter, ventricular fibrillation, ventricular flutter, ventricular tachyarrhythmia, Torsade de pointes tachycardia, atrial and ventricular extrasystoles, AV-junctional extrasystoles, sick sinus syndrome, syncopes, AV-nodal re-entry tachycardia, Wolff-Parkinson-White syndrome, of acute coronary syndrome (ACS), autoimmune cardiac disorders (pericarditis, endocarditis, valvolitis, aortitis, cardiomyopathies), shock such as cardiogenic shock, septic shock and anaphylactic shock, aneurysms, boxer cardiomyopathy (premature ventricular contraction (PVC)), for the treatment and/or prophylaxis of thromboembolic disorders and ischemias such as myocardial ischemia, myocardial infarction, stroke, cardiac hypertrophy, transient and ischemeic attacks, preeclampsia, inflammatory cardiovascular disorders, spasms of the coronary arteries and peripheral arteries, edema formation such as, for example, pulmonary edema, cerebral edema, renal edema or edema caused by heart failure, peripheral circulatory disturbances, reperfusion damage, arterial and venous thromboses, microalbuminuria, myocardial insufficiency, endothelial dysfunction, to prevent restenoses, for example after thrombolysis therapies, percutaneous transluminal angioplasties (PTA), transluminal coronary angioplasties (PTCA), heart transplants and bypass operations, and also micro- and macrovascular damage (vasculitis), increased levels of fibrinogen and of low-density lipoprotein (LDL) and increased concentrations of plasminogen activator inhibitor 1 (PAI-1), and also for the treatment and/or prophylaxis of erectile dysfunction and female sexual dysfunction.

In the context of the present invention, the term “heart failure” encompasses both acute and chronic manifestations of heart failure, and also more specific or related types of disease, such as acute decompensated heart failure, right heart failure, left heart failure, global failure, ischemeic cardiomyopathy, dilated cardiomyopathy, hypertrophic cardiomyopathy, idiopathic cardiomyopathy, congenital heart defects, heart failure associated with heart valve defects, mitral valve stenosis, mitral valve insufficiency, aortic valve stenosis, aortic valve insufficiency, tricuspid valve stenosis, tricuspid valve insufficiency, pulmonary valve stenosis, pulmonary valve insufficiency, combined heart valve defects, myocardial inflammation (myocarditis), chronic myocarditis, acute myocarditis, viral myocarditis, diabetic heart failure, alcoholic cardiomyopathy, cardiac storage disorders, diastolic heart failure and systolic heart failure and acute phases of worsening of existing chronic heart failure (worsening heart failure).

In addition, the compounds of the invention can also be used for the treatment and/or prophylaxis of arteriosclerosis, impaired lipid metabolism, hypolipoproteinemias, dyslipidemias, hypertriglyceridemias, hyperlipidemias, hypercholesterolemias, abetelipoproteinemia, sitosterolemia, xanthomatosis, Tangier disease, adiposity, obesity and of combined hyperlipidemias and metabolic syndrome.

The compounds of the invention can also be used for the treatment and/or prophylaxis of primary and secondary Raynaud's phenomenon, microcirculation impairments, claudication, peripheral and autonomic neuropathies, diabetic microangiopathies, diabetic retinopathy, diabetic ulcers on the extremities, gangrene, CREST syndrome, erythematosis, onychomycosis, rheumatic disorders and for promoting wound healing.

The compounds according to the invention are furthermore suitable for treating urological disorders such as, for example, benign prostate syndrome (BPS), benign prostate hyperplasia (BPH), benign prostate enlargement (BPE), bladder outlet obstruction (BOO), lower urinary tract syndromes (LUTS, including Feline Urological Syndrome (FUS)), disorders of the urogenital system including neurogenic over-active bladder (OAB) and (IC), incontinence (UI) such as, for example, mixed urinary incontinence, urge urinary incontinence, stress urinary incontinence or overflow urinary incontinence (MUI, UUI, SUI, OUI), pelvic pain, benign and malignant disorders of the organs of the male and female urogenital system.

The compounds of the invention are also suitable for the treatment and/or prophylaxis of kidney disorders, in particular of acute and chronic renal insufficiency and acute and chronic renal failure. In the context of the present invention, the term “renal insufficiency” encompasses both acute and chronic manifestations of renal insufficiency, and also underlying or related renal disorders such as renal hypoperfusion, intradialytic hypotension, obstructive uropathy, glomerulopathies, glomerulonephritis, acute glomerulonephritis, glomerulosclerosis, tubulointerstitial diseases, nephropathic disorders such as primary and congenital kidney disease, nephritis, immunological kidney disorders such as kidney transplant rejection and immunocomplex-induced kidney disorders, nephropathy induced by toxic substances, nephropathy induced by contrast agents, diabetic and non-diabetic nephropathy, pyelonephritis, renal cysts, nephrosclerosis, hypertensive nephrosclerosis and nephrotic syndrome which can be characterized diagnostically, for example by abnormally reduced creatinine and/or water excretion, abnormally elevated blood concentrations of urea, nitrogen, potassium and/or creatinine, altered activity of renal enzymes, for example glutamyl synthetase, altered urine osmolarity or urine volume, elevated microalbuminuria, macroalbuminuria, lesions on glomerulae and arterioles, tubular dilatation, hyperphosphatemia and/or need for dialysis. The present invention also encompasses the use of the compounds of the invention for the treatment and/or prophylaxis of sequelae of renal insufficiency, for example pulmonary edema, heart failure, uremia, anemia, electrolyte disorders (for example hyperkalemia, hyponatremia) and disorders in bone and carbohydrate metabolism.

In addition, the compounds of the invention are also suitable for the treatment and/or prophylaxis of asthmatic disorders, pulmonary arterial hypertension (PAH) and other forms of pulmonary hypertension (PH) including left-heart disease-, HIV-, sickle cell anemia-, thromboembolism-(CTEPH), sarcoidosis-, COPD- or pulmonary fibrosis-associated pulmonary hypertension, chronic-obstructive pulmonary disease (COPD), acute respiratory distress syndrome (ARDS), acute lung injury (ALI), alpha-1-antitrypsin deficiency (AATD), pulmonary fibrosis, pulmonary emphysema (for example pulmonary emphysema induced by cigarette smoke) and cystic fibrosis (CF).

The compounds described in the present invention are also active compounds for control of central nervous system disorders characterized by disturbances of the NO/cGMP system. They are suitable in particular for improving perception, concentration, learning or memory after cognitive impairments like those occurring in particular in association with situations/diseases/syndromes such as mild cognitive impairment, age-associated learning and memory impairments, age-associated memory losses, vascular dementia, craniocerebral trauma, stroke, dementia occurring after strokes (post-stroke dementia), post-traumatic craniocerebral trauma, general concentration impairments, concentration impairments in children with learning and memory problems, Alzheimer's disease, Lewy body dementia, dementia with degeneration of the frontal lobes including Pick's syndrome, Parkinson's disease, progressive nuclear palsy, dementia with corticobasal degeneration, amyolateral sclerosis (ALS), Huntington's disease, demyelinization, multiple sclerosis, thalamic degeneration, Creutzfeldt-Jakob dementia, HIV dementia, schizophrenia with dementia or Korsakoffs psychosis. They are also suitable for the treatment and/or prophylaxis of central nervous system disorders such as states of anxiety, tension and depression, CNS-related sexual dysfunctions and sleep disturbances, and for controlling pathological disturbances of the intake of food, stimulants and addictive substances.

In addition, the compounds of the invention are also suitable for controlling cerebral blood flow and are effective agents for controlling migraine. They are also suitable for the prophylaxis and control of sequelae of cerebral infarct (Apoplexia cerebri) such as stroke, cerebral ischemias and skull-brain trauma. The compounds according to the invention can likewise be used for controlling states of pain and tinnitus.

In addition, the compounds of the invention have anti-inflammatory action and can therefore be used as anti-inflammatory agents for the treatment and/or prophylaxis of sepsis (SIRS), multiple organ failure (MODS, MOF), inflammatory disorders of the kidney, chronic intestinal inflammations (IBD, Crohn's disease, UC), pancreatitis, peritonitis, rheumatoid disorders, inflammatory skin disorders and inflammatory eye disorders.

Furthermore, the compounds of the invention can also be used for the treatment and/or prophylaxis of autoimmune diseases.

The compounds of the invention are also suitable for the treatment and/or prophylaxis of fibrotic disorders of the internal organs, for example the lung, the heart, the kidney, the bone marrow and in particular the liver, and also dermatological fibroses and fibrotic eye disorders. In the context of the present invention, the term fibrotic disorders includes in particular the following terms: hepatic fibrosis, cirrhosis of the liver, pulmonary fibrosis, endomyocardial fibrosis, nephropathy, glomerulonephritis, interstitial renal fibrosis, fibrotic damage resulting from diabetes, bone marrow fibrosis and similar fibrotic disorders, scleroderma, morphea, keloids, hypertrophic scarring (also following surgical procedures), naevi, diabetic retinopathy, proliferative vitroretinopathy and disorders of the connective tissue (for example sarcoidosis).

The compounds of the invention are also suitable for controlling postoperative scarring, for example as a result of glaucoma operations.

The compounds of the invention can also be used cosmetically for ageing and keratinizing skin.

Moreover, the compounds according to the invention are suitable for the treatment and/or prophylaxis of hepatitis, neoplasms, osteoporosis, glaucoma and gastroparesis.

The present invention further provides for the use of the compounds according to the invention for the treatment and/or prophylaxis of disorders, especially the disorders mentioned above.

The present invention further provides for the use of the compounds of the invention for the treatment and/or prophylaxis of heart failure, angina pectoris, hypertension, pulmonary hypertension, ischemias, vascular disorders, renal insufficiency, thromboembolic disorders, fibrotic disorders and arteriosclerosis.

The present invention further provides the compounds of the invention for use in a method for the treatment and/or prophylaxis of heart failure, angina pectoris, hypertension, pulmonary hypertension, ischemias, vascular disorders, renal insufficiency, thromboembolic disorders, fibrotic disorders and arteriosclerosis.

The present invention further provides for the use of the compounds of the invention for production of a medicament for the treatment and/or prophylaxis of disorders, especially the aforementioned disorders.

The present invention further provides for the use of the compounds of the invention for production of a medicament for the treatment and/or prophylaxis of heart failure, angina pectoris, hypertension, pulmonary hypertension, ischemias, vascular disorders, renal insufficiency, thromboembolic disorders, fibrotic disorders and arteriosclerosis.

The present invention further provides a method for the treatment and/or prophylaxis of disorders, in particular the disorders mentioned above, using an effective amount of at least one of the compounds of the invention.

The present invention further provides a method for the treatment and/or prophylaxis of heart failure, angina pectoris, hypertension, pulmonary hypertension, ischemias, vascular disorders, renal insufficiency, thromboembolic disorders, fibrotic disorders and arteriosclerosis using an effective amount of at least one of the compounds of the invention.

The compounds according to the invention can be used alone or, if required, in combination with other active compounds. The present invention further provides medicaments comprising at least one of the compounds of the invention and one or more further active compounds, especially for the treatment and/or prophylaxis of the aforementioned disorders. Preferred examples of active compounds suitable for combinations include:
organic nitrates and NO donors, for example sodium nitroprusside, nitroglycerin, isosorbide mononitrate, isosorbide dinitrate, molsidomine or SIN-1, and inhaled NO;
compounds which inhibit the breakdown of cyclic guanosine monophosphate (cGMP), for example inhibitors of phosphodiesterases (PDE) 1, 2 and/or 5, especially PDE 5 inhibitors such as sildenafil, vardenafil and tadalafil;
antithrombotic agents, by way of example and with preference from the group of the platelet aggregation inhibitors, the anticoagulants or the profibrinolytic substances;
hypotensive active compounds, by way of example and with preference from the group of the calcium antagonists, angiotensin AII antagonists, ACE inhibitors, endothelin antagonists, renin inhibitors, alpha-receptor blockers, beta-receptor blockers, mineralocorticoid receptor antagonists, and the diuretics; and/or
active compounds altering lipid metabolism, by way of example and with preference from the group of the thyroid receptor agonists, cholesterol synthesis inhibitors such as, by way of example and preferably, HMG-CoA reductase inhibitors or squalene synthesis inhibitors, the ACAT inhibitors, CETP inhibitors, MTP inhibitors, PPAR-alpha, PPAR-gamma and/or PPAR-delta agonists, cholesterol absorption inhibitors, lipase inhibitors, polymeric bile acid adsorbents, bile acid reabsorption inhibitors and lipoprotein(a) antagonists.

Antithrombotic agents are preferably understood to mean compounds from the group of the platelet aggregation inhibitors, the anticoagulants or the profibrinolytic substances.

In a preferred embodiment of the invention, the compounds of the invention are administered in combination with a platelet aggregation inhibitor, by way of example and with preference aspirin, clopidogrel, ticlopidine or dipyridamole.

In a preferred embodiment of the invention, the compounds of the invention are administered in combination with a thrombin inhibitor, by way of example and with preference ximelagatran, dabigatran, melagatran, bivalirudin or clexane.

In a preferred embodiment of the invention, the compounds of the invention are administered in combination with a GPIIb/IIIa antagonist, by way of example and with preference tirofiban or abciximab.

In a preferred embodiment of the invention, the compounds of the invention are administered in combination with a factor Xa inhibitor, by way of example and with preference rivaroxaban (BAY 59-7939), DU-176b, apixaban, otamixaban, fidexaban, razaxaban, fondaparinux, idraparinux, PMD-3112, YM-150, KFA-1982, EMD-503982, MCM-17, MLN-1021, DX 9065a, DPC 906, JTV 803, SSR-126512 or SSR-128428.

In a preferred embodiment of the invention, the compounds of the invention are administered in combination with heparin or with a low molecular weight (LMW) heparin derivative.

In a preferred embodiment of the invention, the compounds of the invention are administered in combination with a vitamin K antagonist, by way of example and with preference coumarin.

Hypotensive agents are preferably understood to mean compounds from the group of the calcium antagonists, angiotensin AII antagonists, ACE inhibitors, endothelin antagonists, renin inhibitors, alpha-receptor blockers, beta-receptor blockers, mineralocorticoid receptor antagonists, and the diuretics.

In a preferred embodiment of the invention, the compounds of the invention are administered in combination with a calcium antagonist, by way of example and with preference nifedipine, amlodipine, verapamil or diltiazem.

In a preferred embodiment of the invention, the compounds according to the invention are administered in combination with an alpha-1-receptor blocker, by way of example and with preference prazosin.

In a preferred embodiment of the invention, the compounds according to the invention are administered in combination with a beta-receptor blocker, by way of example and with preference propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the compounds of the invention are administered in combination with an angiotensin AII antagonist, by way of example and with preference losartan, candesartan, valsartan, telmisartan or embursatan.

In a preferred embodiment of the invention, the compounds of the invention are administered in combination with an ACE inhibitor, by way of example and with preference enalapril, captopril, lisinopril, ramipril, delapril, fosinopril, quinopril, perindopril or trandopril.

In a preferred embodiment of the invention, the compounds of the invention are administered in combination with an endothelin antagonist, by way of example and with preference bosentan, darusentan, ambrisentan or sitaxsentan.

In a preferred embodiment of the invention, the compounds of the invention are administered in combination with a renin inhibitor, by way of example and with preference aliskiren, SPP-600 or SPP-800.

In a preferred embodiment of the invention, the compounds according to the invention are administered in combination with a mineralocorticoid receptor antagonist, by way of example and with preference spironolactone or eplerenone.

In a preferred embodiment of the invention, the compounds according to the invention are administered in combination with a loop diuretic, for example furosemide, torasemide, bumetanide and piretanide, with potassium-sparing diuretics, for example amiloride and triamterene, with aldosterone antagonists, for example spironolactone, potassium canrenoate and eplerenone, and also thiazide diuretics, for example hydrochlorothiazide, chlorthalidone, xipamide and indapamide.

Lipid metabolism modifiers are preferably understood to mean compounds from the group of the CETP inhibitors, thyroid receptor agonists, cholesterol synthesis inhibitors such as HMG-CoA reductase inhibitors or squalene synthesis inhibitors, the ACAT inhibitors, MTP inhibitors, PPAR-alpha, PPAR-gamma and/or PPAR-delta agonists, cholesterol absorption inhibitors, polymeric bile acid adsorbers, bile acid reabsorption inhibitors, lipase inhibitors and the lipoprotein(a) antagonists.

In a preferred embodiment of the invention, the compounds according to the invention are administered in combination with a CETP inhibitor, by way of example and with preference dalcetrapib, BAY 60-5521, anacetrapib or CETP vaccine (CETi-1).

In a preferred embodiment of the invention, the compounds of the invention are administered in combination with a thyroid receptor agonist, by way of example and with preference D-thyroxine, 3,5,3′-triiodothyronine (T3), CGS 23425 or axitirome (CGS 26214).

In a preferred embodiment of the invention, the compounds of the invention are administered in combination with an HMG-CoA reductase inhibitor from the class of statins, by way of example and with preference lovastatin, simvastatin, pravastatin, fluvastatin, atorvastatin, rosuvastatin or pitavastatin.

In a preferred embodiment of the invention, the compounds of the invention are administered in combination with a squalene synthesis inhibitor, by way of example and with preference BMS-188494 or TAK-475.

In a preferred embodiment of the invention, the compounds of the invention are administered in combination with an ACAT inhibitor, by way of example and with preference avasimibe, melinamide, pactimibe, eflucimibe or SMP-797.

In a preferred embodiment of the invention, the compounds of the invention are administered in combination with an MTP inhibitor, by way of example and with preference implitapide, BMS-201038, R-103757 or JTT-130.

In a preferred embodiment of the invention, the compounds according to the invention are administered in combination with a PPAR-gamma agonist, by way of example and with preference pioglitazone or rosiglitazone.

In a preferred embodiment of the invention, the compounds according to the invention are administered in combination with a PPAR-delta agonist, by way of example and with preference GW 501516 or BAY 68-5042.

In a preferred embodiment of the invention, the compounds of the invention are administered in combination with a cholesterol absorption inhibitor, by way of example and with preference ezetimibe, tiqueside or pamaqueside.

In a preferred embodiment of the invention, the compounds of the invention are administered in combination with a lipase inhibitor, by way of example and with preference orlistat.

In a preferred embodiment of the invention, the compounds of the invention are administered in combination with a polymeric bile acid adsorber, by way of example and with preference cholestyramine, colestipol, colesolvam, CholestaGel or colestimide.

In a preferred embodiment of the invention, the compounds of the invention are administered in combination with a bile acid reabsorption inhibitor, by way of example and with preference ASBT (=IBAT) inhibitors, for example AZD-7806, S-8921, AK-105, BARI-1741, SC-435 or SC-635.

In a preferred embodiment of the invention, the compounds of the invention are administered in combination with a lipoprotein(a) antagonist, by way of example and with preference gemcabene calcium (CI-1027) or nicotinic acid.

The present invention further provides medicaments which comprise at least one compound of the invention, typically together with one or more inert, non-toxic, pharmaceutically suitable excipients, and for the use thereof for the aforementioned purposes.

The compounds of the invention can act systemically and/or locally. For this purpose, they can be administered in a suitable manner, for example by the oral, parenteral, pulmonal, nasal, sublingual, lingual, buccal, rectal, dermal, transdermal, conjunctival or otic route, or as an implant or stent.

The compounds of the invention can be administered in administration forms suitable for these administration routes.

Suitable administration forms for oral administration are those which work according to the prior art and release the compounds according to the invention rapidly and/or in a modified manner and which contain the compounds according to the invention in crystalline and/or amorphized and/or dissolved form, for example tablets (uncoated or coated tablets, for example with gastric juice-resistant or retarded-dissolution or insoluble coatings which control the release of the compound of the invention), tablets or films/oblates which disintegrate rapidly in the oral cavity, films/lyophilizates, capsules (for example hard or soft gelatin capsules), sugar-coated tablets, granules, pellets, powders, emulsions, suspensions, aerosols or solutions.

Parenteral administration can be accomplished with avoidance of a resorption step (for example by an intravenous, intraarterial, intracardiac, intraspinal or intralumbar route) or with inclusion of a resorption (for example by an intramuscular, subcutaneous, intracutaneous, percutaneous or intraperitoneal route). Administration forms suitable for parenteral administration include preparations for injection and infusion in the form of solutions, suspensions, emulsions, lyophilizates or sterile powders.

For the other administration routes, suitable examples are inhalable medicament forms (including powder inhalers, nebulizers), nasal drops, solutions or sprays, tablets, films/oblates or capsules for lingual, sublingual or buccal administration, suppositories, ear or eye preparations, vaginal capsules, aqueous suspensions (lotions, shaking mixtures), lipophilic suspensions, ointments, creams, transdermal therapeutic systems (e.g. patches), milk, pastes, foams, sprinkling powders, implants or stents.

Preference is given to oral or parenteral administration, especially oral administration.

The compounds of the invention can be converted to the administration forms mentioned. This can be accomplished in a manner known per se by mixing with inert, non-toxic, pharmaceutically suitable excipients. These excipients include carriers (for example microcrystalline cellulose, lactose, mannitol), solvents (e.g. liquid polyethylene glycols), emulsifiers and dispersing or wetting agents (for example sodium dodecylsulfate, polyoxysorbitan oleate), binders (for example polyvinylpyrrolidone), synthetic and natural polymers (for example albumin), stabilizers (e.g. antioxidants, for example ascorbic acid), colorants (e.g. inorganic pigments, for example iron oxides) and flavor and/or odor correctants.

In general, it has been found to be advantageous in the case of parenteral administration to administer amounts of about 0.001 to 1 mg/kg, preferably about 0.01 to 0.5 mg/kg, of body weight to achieve effective results. In the case of oral administration, the dose is about 0.001 to 2 mg/kg, preferably about 0.001 to 1 mg/kg, of body weight.

It may nevertheless be necessary in some cases to deviate from the stated amounts, specifically as a function of body weight, route of administration, individual response to the active ingredient, nature of the preparation and time or interval over which administration takes place. Thus in some cases it may be sufficient to manage with less than the abovementioned minimum amount, while in other cases the upper limit mentioned must be exceeded. In the case of administration of greater amounts, it may be advisable to divide them into several individual doses over the day.

The working examples which follow illustrate the invention. The invention is not restricted to the examples.

Unless stated otherwise, the percentages in the tests and examples which follow are percentages by weight; parts are parts by weight. Solvent ratios, dilution ratios and concentration data for liquid/liquid solutions are based in each case on volume.

### A. EXAMPLES

Abbreviations and acronyms:

| | |
|---|---|
| abs. | absolute (=dried) |
| aq. | aqueous solution |
| Boc | tert-butyloxycarbonyl |
| br. | broad signal (NMR coupling pattern) |
| CAS No. | Chemical Abstracts Service number |
| Cbz | benzyloxycarbonyl |
| δ | shift in the NMR spectrum (stated in ppm) |
| d | doublet (NMR coupling pattern) |
| TLC | thin-layer chromatography |
| DCI | direct chemical ionization (in MS) |
| DMAP | 4-N,N-dimethylaminopyridine |
| DMF | dimethylformamide |
| DMSO | dimethyl sulfoxide |
| ent | enantiomerically pure |
| eq. | equivalent(s) |
| ESI | electrospray ionization (in MS) |
| Et | ethyl |
| h | hour(s) |
| HATU | N-[(dimethylamino)(3H-[1,2,3]triazolo[4,5-b]-pyridin-3- |
| | yloxy)methylene]-N-methylmethanaminium |
| | hexafluorophosphate |
| HPLC | high-pressure, high-performance liquid chromatography |
| HRMS | high-resolution mass spectrometry |
| ID | internal diameter |
| conc. | concentrated |
| LC-MS | liquid chromatography-coupled mass spectrometry |
| LiHMDS | lithium hexamethyldisilazide |
| m | multiplet (NMR coupling pattern) |
| Me | methyl |
| min | minute(s) |
| MS | mass spectrometry |
| NMR | nuclear magnetic resonance spectrometry |
| PDA | photodiode array detector |
| Ph | phenyl |
| q | quartet (NMR coupling pattern) |
| quint | quintet (NMR coupling pattern) |
| rac | racemic |
| rel | relative stereochemistry |
| R_{f} | retention factor (in thin-layer chromatography) |
| RT | room temperature |
| Rₜ | retention time (in HPLC) |
| s | singlet (NMR coupling pattern) |
| t | triplet (NMR coupling pattern) |
| THF | tetrahydrofuran |
| TBTU | (benzotriazol-1-yloxy)bisdimethylaminomethylium |
| | fluoroborate |
| UPLC-MS | ultra-pressure liquid chromatography-coupled mass |
| | spectrometry |
| UV | ultraviolet spectrometry |
| vol | Volume |
| v/v | volume to volume ratio (of a solution) |

LC-MS Methods:

Method 1 (Analytical):

Instrument: Acquity UPLC coupled with Quattro Micro mass spectrometer; column: Acquity UPLC BEH C18 (50 mm×2.1 mm ID, 1.7 μm packing diameter); mobile phase A: 10 mM aqueous ammonium bicarbonate solution (adjusted with ammonia to a pH of 10), mobile phase B: acetonitrile; gradient: 0.0 min 97% A, 3% B, flow rate 1 ml/min; 1.5 min 100% B, flow rate 1 ml/min; 1.9 min 100% B, flow rate 1 ml/min; 2.0 min 97% A, 3% B, flow rate 0.05 ml/min; column temperature: 40° C.; UV detection: from 210 nm to 350 nm; MS conditions: ionization mode: alternating scans positive and negative electrospray (ES+/ES−); scan range: 100 to 1000 AMU.

Method 2 (Analytical):

Instrument: Acquity UPLC coupled with Quattro Micro mass spectrometer; column: Acquity UPLC BEH C18 (50 mm×2.1 mm ID, 1.7 μm packing diameter); mobile phase A: 0.1% by volume solution of formic acid in water, mobile phase B: 0.1% by volume solution of formic acid in acetonitrile, gradient: 0.0 min 97% A, 3% B, flow rate 1 ml/min; 1.5 min 100% B, flow rate 1 ml/min; 1.9 min 100% B, flow rate 1 ml/min; 2.0 min 97% A, 3% B, flow rate 0.05 ml/min; column temperature: 40° C.; UV detection: from 210 nm to 350 nm; MS conditions: ionization mode: alternating scans positive and negative electrospray (ES+/ES−); scan range: 100 to 1000 AMU.

Method 3 (Analytical):

Instrument: Waters 2690, PDA detector Waters 2996 coupled with Quattro Micro mass MS detector; column: Waters SunFire C18 3.5 μm, 2.1×50 mm; mobile phase A: 0.1% formic acid in water, mobile phase B: 0.1% formic acid in acetonitrile; gradient: 0.0 min 95% A, 5% B, flow rate 0.5 ml/min; 3.0 min 95% A, 5% B, flow rate 0.5 ml/min; 17.50 min 5% A, 95% B, flow rate 0.5 ml/min; 19.00 min 5% A, 95% B, flow rate 0.5 ml/min; 19.50 min 95% A, 5% B, flow rate 0.5 ml/min; 20.00 min 95% A, 5% B, flow rate 0.5 ml/min; column temperature: 30° C.; UV detection: from 210 nm to 400 nm; MS conditions: ionization mode: scans positive and negative electrospray (ES+/ES−); scan range: 130 to 1100 AMU.

Method 4 (Preparative):

Instrument: Waters Mass Directed AutoPurification System, LC-MS system: MDAP system fitted with ZQ mass spectrometer; column: XBridge Prep. MS C18 OBD (150 mm×30 mm ID 5 particle size); mobile phase A: 10 mM ammonium bicarbonate (adjusted with ammonia to a pH of 10), mobile phase B: acetonitrile; gradient: 0.0 min 97% A, 3% B, flow rate 50 ml/min; 1.0 min 70% A, 30% B, flow rate 50 ml/min; 10 min 20% A, 80% B, flow rate 50 ml/min; 10.5 min 0%

A, 100% B, flow rate 50 ml/min; 15 min 0% A, 100% B, flow rate 50 ml/min; UV detection: from 210 nm to 600 nm; MS conditions: ionization mode: scans positive and negative electrospray (ES+/ES−); scan range: 100 to 1000 AMU.

Method 5 (Analytical):

Instrument: Waters ACQUITY SQD UPLC system; column: Waters Acquity UPLC HSS T3 1.8μ 50×1 mm; mobile phase A: 1 1 of water+0.25 ml of 99% strength formic acid; mobile phase B: 1 1 of acetonitrile+0.25 ml of 99% strength formic acid; gradient: 0.0 min 90% A→1.2 min 5% A→2.0 min 5% A; oven: 50° C.; flow rate: 0.40 ml/min; UV detection: 210-400 nm.

Method 6 (LC-MS):

Instrument: Micromass Quattro Premier with Waters UPLC Acquity; column: Thermo Hypersil GOLD 1.9μ 50×1 mm; mobile phase A: 1 1 of water+0.5 ml of 50% strength formic acid, mobile phase B: 1 1 of acetonitrile+0.5 ml of 50% strength formic acid; gradient: 0.0 min 97% A→0.5 min 97% A→3.2 min 5% A→4.0 min 5% A; oven: 50° C.; flow rate: 0.3 ml/min; UV detection: 210 nm.

Method 7 (LC-MS):

MS instrument: Waters (Micromass) QM; HPLC instrument: Agilent 1100 series; column: Agient ZORBAX Extend-C18 3.0×50 mm 3.5 micron; mobile phase A: 1 1 of water+0.01 mol of ammonium carbonate, mobile phase B: 1 1 of acetonitrile; gradient: 0.0 min 98% A→0.2 min 98% A→3.0 min 5% A→4.5 min 5% A; oven: 40° C.; flow rate: 1.75 ml/min; UV detection: 210 nm

Method 8 (LC-MS):

Instrument: Waters ACQUITY SQD UPLC system; column: Waters Acquity UPLC HSS T3 1.8μ 30×2 mm; mobile phase A: 1 1 of water+0.25 ml of 99% strength formic acid, mobile phase B: 1 1 of acetonitrile+0.25 ml of 99% strength formic acid; gradient: 0.0 min 90% A→1.2 min 5% A→2.0 min 5% A; oven: 50° C.; flow rate: 0.60 ml/min; UV detection: 208-400 nm.

Method 9 (GC-MS):

Instrument: Theiiiio Scientific DSQII, Thermo Scientific Trace GC Ultra; column: Restek RTX-35MS, 15 m×200 μm×0.33 μm; constant flow rate with helium: 1.20 ml/min; oven: 60° C.; inlet: 220° C.; gradient: 60° C., 30° C./min→300° C. (maintain for 3.33 min).

Method 10 (MS):

Instrument: Waters ZQ 2000; electrospray ionization; mobile phase A: 1 1 of water+0.25 ml of 99% strength formic acid, mobile phase B: 1 1 of acetonitrile+0.25 ml of 99% strength formic acid; 25% A, 75% B; flow rate: 0.25 ml/min.

Method 11 (LC-MS):

Instrument: Agilent MS Quad 6150; HPLC: Agilent 1290; column: Waters Acquity UPLC HSS T3 1.8μ 50×2.1 mm; mobile phase A: 1 1 of water+0.25 ml of 99% strength formic acid, mobile phase B: 1 1 of acetonitrile+0.25 ml of 99% strength formic acid; gradient: 0.0 min 90% A→0.3 min 90% A→1.7 min 5% A→3.0 min 5% A oven: 50° C.; flow rate: 1.20 ml/min; UV detection: 205-305 nm.

Method 12 (Preparative HPLC):

MS instrument: Waters, HPLC instrument: Waters (column Waters X-Bridge C18, 19 mm×50 mm, 5 μm, mobile phase A: water+0.05% ammonia, mobile phase B: acetonitrile (ULC) with gradient; flow rate: 40 ml/min; UV detection: DAD; 210-400 nm).

or:

MS instrument: Waters, HPLC instrument: Waters (column Phenomenex Luna 5μ C18(2) 100 A, AXIA Tech. 50×21.2 mm, mobile phase A: water+0.05% formic acids, mobile phase B: acetonitrile (ULC) with gradient; flow rate: 40 ml/min; UV detection: DAD; 210-400 nm).

Method 13 (LC-MS):

MS instrument: Waters SQD; HPLC instrument: Waters UPLC; column: Zorbax SB-Aq (Agilent), 50 mm×2.1 mm, 1.8 μm; mobile phase A: water+0.025% formic acid, mobile phase B: acetonitrile (ULC)+0.025% formic acid; gradient: 0.0 min 98% A-0.9 min 25% A-1.0 min 5% A-1.4 min 5% A-1.41 min 98% A-1.5 min 98% A; oven: 40° C.; flow rate: 0,600 ml/min; UV detection: DAD; 210 nm.

Method 14: SYNCOM

MS instrument type: HP 6130 MSD; HPLC instrument type: Agilent 1290 series; UV DAD; column: Waters XBridge BEH C18 2.5 μm 2.1 mm×50 mm; mobile phase A: ammonium acetate (10 mM)+water/methanol/acetonitrile (9.0:0.6:0.4), mobile phase B: ammonium acetate (10 mM)+water/methanol/acetonitrile (1.0:5.4:3.6), gradient: A/B: 100/0 (1.0 min) (1.0 min)→40/60 (0.0 min) (0.5 min)→0/100 (1.0 min); flow rate: 0.6 ml/min; oven: 35° C.; UV detection: 215 and 254 nm.

Starting Compounds and Intermediates:

### Example 1A

### 4-Bromoquinolin-8-ol

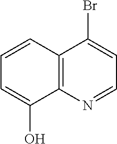 [SMILES: Oc1cccc2c(Br)ccnc12]

1.5 g (0.006 mol) of 4-bromo-8-methoxyquinoline (CAS No.: 103028-31-5) were dissolved in 10 ml 48% strength aqueous hydrobromic acid (CAS No.: 10035-10-6). The reaction mixture was stirred at reflux for 24 hours. Water (50 ml) was added and the solution was neutralized with 2M aqueous sodium hydroxide solution and extracted with dichloromethane (2×50 ml). The organic extracts were combined, dried over magnesium sulfate and concentrated under reduced pressure. The residue was purified by flash chromatography using a silica gel cartridge [mobile phase: dichloromethane:/(dichloromethane:methanol 90:5), gradient 5% to 15%]. This gave 0.84 g (59% of theory) of the target product.

LC-MS (Method 2): Rₜ=0.94 min; m/z=225.09 (M+H)⁺

¹H-NMR (300 MHz, DMSO-d₆): δ [ppm]=7.16-7.19 (m, 1H), 7.51-7.61 (m, 2H), 7.93 (d, 1H), 8.64 (d, 1H), 10.09 (s, 1H).

### Example 2A

### 4-Bromo-8-[(2,6-difluorobenzyl)oxy]quinoline

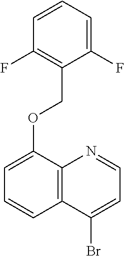 [SMILES: Fc1cccc(F)c1COc1cccc2c(Br)ccnc12]

1.75 g (0.005 mol) of cesium carbonate were added to a solution, stirred vigorously, of 1.22 g (0.006 mol) of 2,6-difluorobenzyl bromide (CAS No.: 85118-00-9) and 1.2 g (0.005 mol) of 4-bromoquinolin-8-ol (Example 1A) in 25 ml of N,N′-dimethylformamide. The reaction mixture was stirred for 4 hours at room temperature. After concentration under reduced pressure, water was added and the mixture was extracted three times with dichloromethane. The organic phase was dried over magnesium sulfate and concentrated to dryness, which gave 1.87 g (98% of theory, purity 98%) of the target product, which was used without further purification.

LC-MS (Method 2): Rₜ=1.24 min; m/z=352.06 (M+H)⁺

¹H-NMR (300 MHz, DMSO-d₆): δ [ppm]=5.32 (s, 2H), 7.18-7.26 (m, 2H), 7.50-7.62 (m, 2H), 7.67-7.76 (m, 2H), 7.94 (d, 1H), 8.62 (d, 1H).

### Example 3A

### 8-[(2,6-Difluorobenzyl)oxy]quinoline-4-carbonitrile

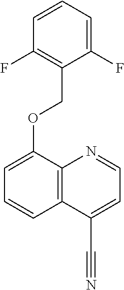 [SMILES: N#Cc1ccnc2c(OCc3c(F)cccc3F)cccc12]

1.87 g (0.005 mol) of 4-bromo-8-[(2,6-difluorobenzyl)oxy]quinoline (Example 2A) and 2.35 g (0.026 mol) copper(I) cyanide (CAS No.: 544-92-3) were dissolved in 46 ml of dimethyl sulfoxide. The reaction mixture was heated in a microwave at 160° C. for 60 minutes. The reaction mixture was diluted with ethyl acetate and the mixture was extracted with saturated aqueous ammonium chloride solution and aqueous sodium chloride solution. The organic phase was dried over sodium sulfate and concentrated under reduced pressure, which gave 1.4 g (75% of theory, purity 83%) of the target compound.

LC-MS (Method 2): Rₜ=1.13 min; m/z=297.25 (M+H)⁺

¹H-NMR (300 MHz, DMSO-d₆): δ [ppm]=5.35 (s, 2H), 7.03-7.40 (m, 3H), 7.55-7.72 (m, 2H), 7.79-7.82 (m, 2H), 8.15 (br. s, 1H).

### Example 4A

### 8-[(2,6-Difluorobenzyl)oxy]quinoline-4-carboxylic acid

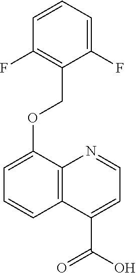 [SMILES: O=C(O)c1ccnc2c(OCc3c(F)cccc3F)cccc12]

1.16 g (0.004 mol) of 8-[(2,6-difluorobenzyl)oxy]quinoline-4-carbonitrile (Example 3A) were dissolved in 90 ml of ethanol. 3.14 g (0.078 mol) of aqueous sodium hydroxide solution were added to the solution. The reaction mixture was heated at reflux for 18 hours. 70 ml of water were added to the reaction mixture and the pH of the solution was adjusted to 2 using 1N aqueous hydrochloric acid. The precipitate was filtered off and dried under reduced pressure, which gave 1.2 g (80% of theory, purity 83%) of the target product.

LC-MS (Method 2): Rₜ=0.67 min; m/z=316.21 (M+H)⁺

¹H-NMR (300 MHz, DMSO-d₆): δ [ppm]=5.33 (s, 2H), 7.21 (m, 2H), 7.45-7.52 (m, 1H), 7.53-7.61 (m, 1H), 7.63-7.75 (m, 1H), 7.94 (d, 1H), 8.24 (d, 1H), 8.95 (d, 1H).

### Example 5A

### (2S)-Benzyl 1-amino-2-methylbutan-2-yl)carbamate

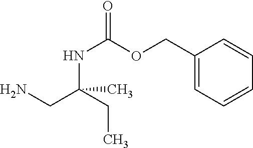 [SMILES: CC[C@@](C)(CN)NC(=O)OCc1ccccc1]

10 ml of Raney nickel (50% suspension in water) were added to a solution of 6.0 g (0.026 mol) of benzyl (25)-2-cyanobutan-2-ylcarbamate (Example 8A) in 75 ml of a 7M solution of ammonia in methanol. For 18 hours, the reaction mixture was kept in an autoclave at room temperature and a hydrogen pressure of 25 bar. The reaction mixture was filtered through a layer of Celite which was washed with methanol (50 ml), and the combined filtrates were concentrated under reduced pressure. The residue was purified by flash chromatography using a silica gel cartridge [mobile phase: dichloromethane/(dichloromethane:methanol: 2M ammonia in methanol 95:5:5), gradient 5% to 15%], which gave 2.81 g (41% of theory, purity 95%) of the target product.

LC-MS (Method 3): Rₜ=4.46 min; m/z=237.10 (M+H)⁺

¹H-NMR (300 MHz, DMSO-d₆): δ [ppm]=0.73 (t, 3H), 1.08 (s, 3H), 1.38-1.68 (m, 4H), 2.43-2.50 (m, 2H), 4.96 (s, 2 H), 6.63 (br. s, 1H), 7.25-7.38 (m, 5H).

### Example 6A

### (2S)-Benzyl 1-[({8-(2,6-difluorobenzyl)oxy]quinolin-4-yl}carbonyl)amino]-2-methylbutan-2-yl}carbamate

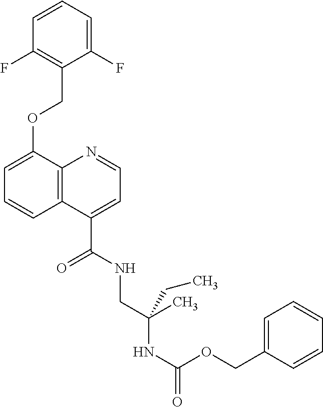 [SMILES: CC[C@@](C)(CNC(=O)c1ccnc2c(OCc3c(F)cccc3F)cccc12)NC(=O)OCc1ccccc1]

64 mg (0.397 mmol) of 1,1′-carbonyldiimidazole and 33 mg (0.214 mmol) of 1-hydroxybenzotriazole were added to a solution of 96 mg (0.305 mmol) of 8-[(2,6-difluorobenzyl)oxy]quinoline-4-carboxylic acid (Example 4A) in 3.5 ml of N,N′-dimethylformamide. The mixture was stirred at room temperature for a further 15 minutes. 94 mg (0.397 mmol) of benzyl (2S)-1-amino-2-methylbutan-2-yl)carbamate (Example 5A) and 0.106 ml of N,N-diisopropylethylamine were added to the reaction mixture. The reaction mixture was heated in a microwave at 100° C. for 20 min and then allowed to cool and concentrated under reduced pressure. The residue was partitioned between water and dichloromethane and the aqueous phase was extracted twice with dichloromethane. The combined organic phases were washed with saturated aqueous sodium bicarbonate solution, dried over sodium sulfate and concentrated under reduced pressure. The residue was purified by preparative HPLC chromatography (Method 4), which gave 51 mg (31% of theory) of the target product.

LC-MS (Method 2): Rₜ=1.19 min; m/z=534.36 (M+H)⁺

¹H-NMR (300 MHz, DMSO-d₆): δ [ppm]=0.82 (t, 3H), 1.20 (s, 3H), 1.47-1.60 (m, 2H), 1.80-1.87 (m, 1H), 3.47-3.61 (m, 2H), 4.98 (s, 2H), 5.32 (s, 2 H), 7.24 (t, 2H), 7.29-7.36 (m, 3H), 7.41 (d, 1H), 7.51-7.67 (m, 6H), 8.66 (t, 1H), 8.85 (d, 1H).

### Example 7A

### rac-Benzyl (2-cyanobutan-2-yl)carbamate

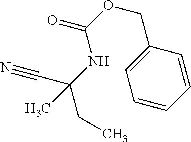 [SMILES: CCC(C)(C#N)NC(=O)OCc1ccccc1]

5.00 g (50.94 mmol) of 2-amino-2-methylbutanonitrile [synthesis described in: Lonza AG, U.S. Pat. No. 5,698,704 (1997); Deng, S. L. et al. Synthesis 2001, 2445; Hjorringgaard, C. U. et al. J. Org. Chem. 2009, 74, 1329; Ogrel, A. et al. Eur. J Org. Chem. 2000, 857] were initially charged in 50 ml of THF and 6.5 ml of water, 21.83 g (157.92 mmol) of potassium carbonate were added and, at 0° C., 7.9 ml (56.04 mmol) of benzyl chlorocarbonate (benzyl chloroformate) were added. After the addition of 8 ml of THF and 3 ml of water, the reaction mixture was stirred overnight, slowly warming to RT. Water was then added, and the mixture was extracted three times with ethyl acetate. The combined organic phases were dried over sodium sulfate and concentrated. The residue was dissolved in diethyl ether and precipitated with petroleum ether. The product was filtered off and the solid was washed with a little petroleum ether and dried under high vacuum. This gave 11.35 g of the target compound (93% of theory, purity 97%).

LC-MS (Method 5): Rₜ=0.97 min

MS (ESpos): m/z=233 (M+H)⁺

¹H NMR (400 MHz, DMSO-d₆): δ=0.95 (t, 3H), 1.51 (s, 3H), 1.75-1.95 (m, 2H), 5.07 (s, 2H), 7.30-7.43 (m, 4H), 7.88-8.03 (m, 1H).

### Example 8A

### ent-Benzyl (2-cyanobutan-2-yl)carbamate (Enantiomer A)

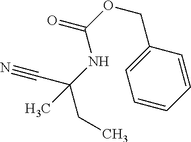 [SMILES: CCC(C)(C#N)NC(=O)OCc1ccccc1]

8 g of rac-benzyl (2-cyanobutan-2-yl)carbamate (Example 7A) were separated into the enantiomers by preparative separation on a chiral phase [column: Daicel Chiralcel OJ-H, 5 μm, 250×20 mm, mobile phase: 50% isohexane, 50% isopropanol, flow rate: 20 ml/min; 40° C., UV detection: 220 nm].

Enantiomer A: yield: 3.23 g (>99% ee)

Rₜ=6.69 min [Daicel Chiralcel OJ-H, 5 μm, 250×4,6 mm; mobile phase: 50% isohexane, 50% isopropanol, flow rate: 1.0 ml/min; 30° C.; detection: 220 nm].

### Example 9A

### rac-2-Amino-5,5,5-trifluoro-2-methylpentanonitrile

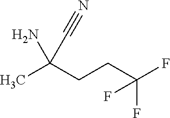

8.0 g (57.1 mmol) of 5,5,5-trifluoropentan-2-one [CAS Registry Number: 1341078-97-4; commercially available, or the methyl ketone can be prepared by literature methods which are known to those skilled in the art, for example via a) two stages from 4,4,4-trifluorobutanal according to Y. Bai et al. Angewandte Chemie 2012, 51, 4112-4116; K. Hiroi et al. Synlett 2001, 263-265; K. Mikami et al. 1982 Chemistry Letters, 1349-1352; b) or from 4,4,4-trifluorobutanoic acid according to A. A. Wube et al. Bioorganic and Medicinal Chemistry 2011, 19, 567-579; G. M. Rubottom et al. Journal of Organic Chemistry 1983, 48, 1550-1552; T. Chen et al. Journal of Organic Chemistry 1996, 61, 4716-4719. The product can be isolated by distillation or chromatography.] were initially charged in 47.8 ml of 2 N ammonia in methanol, 3.69 g (75.4 mmol) of sodium cyanide and 4.03 g (75.4 mmol) of ammonium chloride were added at room temperature and the mixture was stirred under reflux for 4 hours. The reaction mixture was cooled, diethyl ether was added and the solids present were filtered off. The solvent was distilled out of the filtrate under standard pressure. 8.7 g of the title compound (92% of theory) were obtained as residue, which was used in the subsequent stage without further purification.

GC-MS (Method 9): Rₜ=1.90 min

MS (ESpos): m/z=151 (M-CH₃)⁺

### Example 10A

### rac-Benzyl (2-cyano-5,5,5-trifluoropentan-2-yl)carbamate

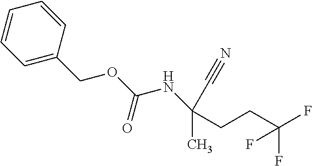 [SMILES: CC(C#N)(CCC(F)(F)F)NC(=O)OCc1ccccc1]

8.7 g (52.36 mmol) of rac-2-amino-5,5,5-trifluoro-2-methylpentanonitrile from Example 9A were initially charged in 128 ml of tetrahydrofuran/water=9/1, and 22.43 g (162.3 mmol) of potassium carbonate were added. At 0° C., 8.93 g (52.36 mmol) of benzyl chloroformate were slowly added dropwise. Then the mixture was allowed to warm up gradually to room temperature with stirring, and was stirred at room temperature overnight. The supernatant solvent was decanted off, the residue was twice stirred with 100 ml each time of tetrahydrofuran, and then the supernatant solvent was decanted off each time. The combined organic phases were concentrated and the crude product was purified by silica gel chromatography (mobile phase: cyclohexane/ethyl acetate gradient 9/1 to 4/1). This gave 11.14 g of the title compound (68% of theory).

LC-MS (Method 5): Rₜ=1.01 min

MS (ESpos): m/z=301 (M+H)⁺

¹H-NMR (400 MHz, DMSO-dₒ): δ [ppm]=1.58 (s, 3H), 2.08-2.21 (m, 2H), 2.24-2.52 (m, 2H), 5.09 (s, 2H), 7.29-7.41 (m, 5H), 8.17 (br. s, 1H).

### Example 11A

### ent-Benzyl (2-cyano-5,5,5-trifluoropentan-2-yl)carbamate (Enantiomer A)

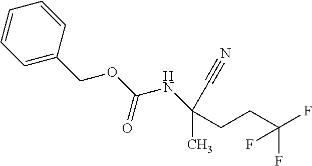 [SMILES: CC(C#N)(CCC(F)(F)F)NC(=O)OCc1ccccc1]

11.14 g of rac-benzyl (2-cyano-5,5,5-trifluoropentan-2-yl)carbamate from Example 10A were separated into the enantiomers by preparative separation on a chiral phase [column: Daicel Chiralpak AZ-H, 5 SFC, 250×50 mm, mobile phase: 94% carbon dioxide, 6% methanol, flow rate: 200 ml/min, temperature: 38° C., pressure: 135 bar; detection: 210 nm].

Enantiomer A: 4.12 g (about 79% ee)

Rₜ=1.60 min [SFC, Daicel Chiralpak AZ-H, 250×4.6 mm, 5 μm, mobile phase: 90% carbon dioxide, 10% methanol, flow rate: 3 ml/min, temperature: 30° C., detection: 220 nm].

LC-MS (Method 5): Rₜ=1.01 min

MS (ESpos): m/z=301 (M+H)⁺

### Example 12A

### ent-Benzyl (2-cyano-5,5,5-trifluoropentan-2-yl)carbamate (Enantiomer B)

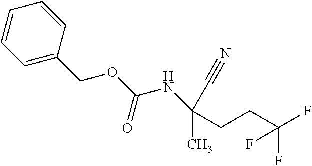 [SMILES: CC(C#N)(CCC(F)(F)F)NC(=O)OCc1ccccc1]

11.14 g of rac-benzyl (2-cyano-5,5,5-trifluoropentan-2-yl)carbamate from Example 10A were separated into the enantiomers by preparative separation on a chiral phase [column: Daicel Chiralpak AZ-H, 5 SFC, 250×50 mm, mobile phase: 94% carbon dioxide, 6% methanol, flow rate: 200 ml/min, temperature: 38° C., pressure: 135 bar; detection: 210 nm].

Enantiomer B: 4.54 g (about 70% ee, purity about 89%)

Rₜ=1.91 min [SFC, Daicel Chiralpak AZ-H, 250×4.6 mm, 5 μm, mobile phase: 90% carbon dioxide, 10% methanol, flow rate: 3 ml/min, temperature: 30° C., detection: 220 nm].

LC-MS (Method 5): Rₜ=1.01 min

MS (ESpos): m/z=301 (M+H)⁺

### Example 13A

### ent-Benzyl (1-amino-5,5,5-trifluoro-2-methylpentan-2-yl)carbamate (Enantiomer A)

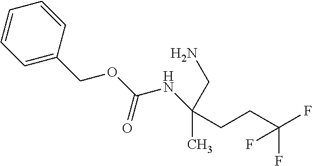 [SMILES: CC(CN)(CCC(F)(F)F)NC(=O)OCc1ccccc1]

4.12 g (13.17 mmol) of ent-benzyl (2-cyano-5,5,5-trifluoropentan-2-yl)carbamate (enantiomer A) from Example 11A were dissolved in 39 ml of 7 N ammonia solution in methanol, and 4 g of Raney nickel (50% aqueous slurry) were added under argon. The reaction mixture was hydrogenated in an autoclave at 20-30 bar overnight. Another 1 g of Raney nickel (50% aqueous slurry) was added and the reaction mixture was hydrogenated in an autoclave at 20-30 bar for 5 h. The reaction mixture was filtered through kieselguhr, rinsed with methanol and concentrated. 3.35 g (56% of theory; purity about 67%) of the target compound were obtained, which were used in the subsequent stage without further purification.

LC-MS (Method 8): Rₜ=1.68 min

MS (ESpos): m/z=305 (M+H)⁺

¹H-NMR (400 MHz, DMSO-d₆): δ [ppm]=1.13 (s, 3H), 1.40 (br. s, 2H), 1.70-1.80 (m, 1H), 1.83-1.95 (m, 1H), 2.08-2.2 (m, 2H), 4.98 (s, 2H), 6.85 (br. s, 1H), 7.28-7.41 (m, 5H).

### Example 14A

### ent-Benzyl (1-amino-5,5,5-trifluoro-2-methylpentan-2-yl)carbamate (Enantiomer B)

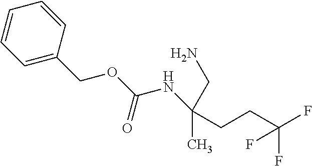 [SMILES: CC(CN)(CCC(F)(F)F)NC(=O)OCc1ccccc1]

4.54 g (13.45 mmol; purity about 89%) of ent-benzyl (2-cyano-5,5,5-trifluoropentan-2-yl)carbamate (enantiomer B) from Example 12A were dissolved in 39 ml of 7 N ammonia solution in methanol, and 5 g of Raney nickel (50% aqueous slurry) were added under argon. The reaction mixture was hydrogenated in an autoclave at 20-30 bar for 3 h. The reaction mixture was filtered through kieselguhr, rinsed with methanol and concentrated. 4.20 g (97% of theory; purity about 95%) of the target compound were obtained, which were used in the subsequent stage without further purification.

LC-MS (Method 7): Rₜ=2.19 min

MS (ESpos): m/z=305 (M+H)⁺

¹H-NMR (400 MHz, DMSO-dₒ): δ [ppm]=1.13 (s, 3H), 1.40 (br. s, 2H), 1.69-1.80 (m, 1H), 1.83-1.96 (m, 1H), 2.07-2.22 (m, 2H), 4.98 (s, 2H), 6.85 (br. s, 1H), 7.27-7.40 (m, 5H).

### Example 15A

### rac-2-[(Diphenylmethylene)amino]-4,4-difluorobutanonitrile

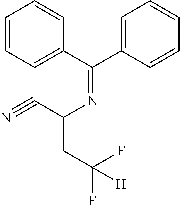 [SMILES: N#CC(CC(F)F)N=C(c1ccccc1)c1ccccc1]

18 g (81.72 mmol) of [(diphenylmethylene)amino]acetonitrile were initially charged in 500 ml of abs. THF, and 39.22 ml (98.06 mmol) of n-butyllithium (2.5 N in hexane) were added at −78° C. under argon, and the mixture was stirred at −78° C. for 15 min. Subsequently, the reaction solution was warmed up to 0° C. 17.25 g (89.89 mmol) of 1,1-difluoro-2-iodoethane were added dropwise to the reaction solution, which was stirred at 0° C. for a further 15 min. At 0° C., water was added to the reaction solution, ethyl acetate was added and the mixture was washed three times with semisaturated aqueous sodium chloride solution. The combined aqueous phases were re-extracted twice with ethyl acetate. The combined organic phases were dried over sodium sulfate, filtered and concentrated. The residue was purified by silica gel chromatography (mobile phase: dichloromethane/cyclohexane =1/1). This gave 13.57 g of the target compound (49% of theory, purity 84%).

LC-MS (Method 6): Rₜ=2.48 min

MS (ESpos): m/z=285 (M+H)⁺

¹H-NMR (400 MHz, DMSO-d₆): δ=2.53-2.61 (m, 2H; partially superposed by solvent peak), 4.50 (t, 1H), 6.08-6.41 (m, 1H), 7.23-7.33 (m, 2H), 7.38-7.47 (m, 2H), 7.49-7.67 (m, 6H).

### Example 16A

### rac-2-[(Diphenylmethylene)amino]-4,4-difluoro-2-methylbutanonitrile

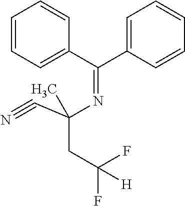 [SMILES: CC(C#N)(CC(F)F)N=C(c1ccccc1)c1ccccc1]

To an initial charge of 13.07 g (38.62 mmol) of rac-2-[(diphenylmethylene)amino]-4,4-difluorobutanonitrile from Example 15A in 255 ml of abs. THF were added 15.6 ml (39.0 mmol) of n-butyllithium (2.5 N in hexane) at −78° C. under argon, and the mixture was stirred at −78° C. for 10 min. Subsequently, 22.6 g (154.46 mmol) of iodomethane were added to the reaction solution at −78° C. The reaction mixture was gradually brought to 0° C. over 3.5 h. After complete reaction of the starting material, water and ethyl acetate were added to the reaction solution at 0° C. and the mixture was washed twice with saturated aqueous sodium chloride solution. The organic phase was dried over sodium sulfate, filtered and concentrated. The residue was purified by silica gel chromatography (mobile phase: cyclohexane/ethyl acetate=15/1). This gave 11.4 g of the target compound (91% of theory, purity 92%).

LC-MS (Method 6): Rₜ=2.52 min

MS (ESpos): m/z=299 (M+H)⁺

¹H-NMR (400 MHz, DMSO-d₆): δ=1.67 (s, 3H), 2.55-2.77 (m, 2H), 6.14-6.48 (m, 1H), 7.28-7.34 (m, 2H), 7.36-7.44 (m, 2H), 7.44-7.54 (m, 6H).

### Example 17A

### rac-2-Amino-4,4-difluoro-2-methylbutanonitrile hydrochloride

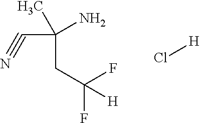

10.84 g (33.43 mmol; 92% purity) of rac-2-[(diphenylmethylene)amino]-4,4-difluoro-2-methylbutanonitrile from Example 16A were dissolved in 156 ml of tetrahydrofuran and 6 ml of water, 73.5 ml (36.77 mmol) of hydrogen chloride solution (0.5 N in diethyl ether) were added and the mixture was stirred at room temperature overnight. 16.71 ml (33.43 mmol) of hydrogen chloride solution (2 N in diethyl ether) were then added to the reaction solution, and the mixture was concentrated. The isolated crude product was reacted further directly without further purification.

LC-MS (Method 6): Rₜ=0.32 min

MS (ESpos): m/z=135 (M-HCl+H)⁺

### Example 18A

### rac-Benzyl (2-cyano-4,4-difluorobutan-2-yl)carbamate

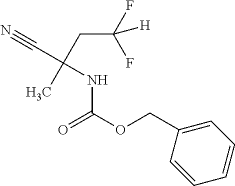 [SMILES: CC(C#N)(CC(F)F)NC(=O)OCc1ccccc1]

The crude product rac-2-amino-4,4-difluoro-2-methylbutanonitrile hydrochloride from Example 17A was initially charged in 109 ml of tetrahydrofuran/water (1:1), and 18.94 g (137.06 mmol) of potassium carbonate and 6.27 g (36.77 mmol) of benzyl chloroformate were added. The reaction mixture was stirred at room temperature overnight. Another 1.14 g (6.69 mmol) of benzyl chloroformate were added to the reaction and the mixture was stirred at room temperature for a further 2 h. Subsequently, the two-phase system was separated and the aqueous phase was extracted twice with ethyl acetate. The combined organic phases were washed once with saturated aqueous sodium chloride solution, and then dried over sodium sulfate, filtered and concentrated. The residue was purified by silica gel chromatography (mobile phase: cyclohexane/ethyl acetate gradient 20/1 to 5/1). This gave 7.68 g of the target compound (61% of theory over two steps, purity 71%).

LC-MS (Method 6): Rₜ=2.04 min

MS (ESpos): m/z=269 (M+H)⁺

¹H-NMR (400 MHz, DMSO-d₆): δ [ppm]=1.65 (s, 3H), 2.51-2.65 (m, 2H), 5.10 (s, 2H), 6.08-6.41 (m, 1H), 7.27-7.44 (m, 5H), 8.24 (br. s, 1H).

### Example 19A

### ent-Benzyl (2-cyano-4,4-difluorobutan-2-yl)carbamate (Enantiomer A)

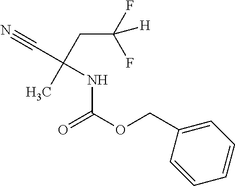 [SMILES: CC(C#N)(CC(F)F)NC(=O)OCc1ccccc1]

7.68 g (20.33 mmol, purity 71%) of rac-benzyl (2-cyano-4,4-difluorobutan-2-yl)carbamate from Example 18A were separated into the enantiomers by preparative separation on the chiral phase [column: Daicel Chiralpak AY-H, 5 μm, 250×20 mm, mobile phase: 80% isohexane, 20% isopropanol; flow rate: 25 ml/min; temperature: 22° C., detection: 210 nm].

Enantiomer A: yield: 2.64 g (>99% ee)

Rₜ=6.67 min [Chiralpak AY-H, 5 μm, 250×4.6 mm; mobile phase: 80% isohexane, 20% isopropanol; flow rate: 3 ml/min; detection: 220 nm].

### Example 20A

### ent-Benzyl (2-cyano-4,4-difluorobutan-2-yl)carbamate (Enantiomer B)

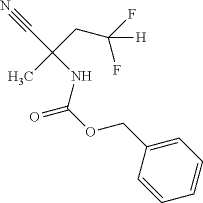 [SMILES: CC(C#N)(CC(F)F)NC(=O)OCc1ccccc1]

7.68 g (20.33 mmol, purity 71%) of rac-benzyl (2-cyano-4,4-difluorobutan-2-yl)carbamate from Example 18A were separated into the enantiomers by preparative separation on the chiral phase [column: Daicel Chiralpak AY-H, 5 μm, 250×20 mm, mobile phase: 80% isohexane, 20% isopropanol; flow rate: 25 ml/min; temperature: 22° C., detection: 210 nm].

Enantiomer B: yield: 2.76 g (93% ee)

Rₜ=7.66 min [Chiralpak AY-H, 5 μm, 250×4.6 mm; mobile phase: 80% isohexane, 20% isopropanol; flow rate: 3 ml/min; detection: 220 nm].

### Example 21A

### ent-Benzyl (1-amino-4,4-difluoro-2-methylbutan-2-yl)carbamate (Enantiomer A)

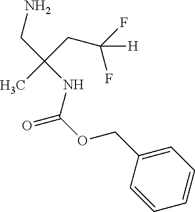 [SMILES: CC(CN)(CC(F)F)NC(=O)OCc1ccccc1]

2.3 g (8.57 mmol) of ent-benzyl (2-cyano-4,4-difluorobutan-2-yl)carbamate (enantiomer A) from Example 19A were dissolved in 75 ml of 7 N ammonia solution in methanol, and 2.66 g of Raney nickel (50% aqueous slurry) were added under argon. The reaction mixture was hydrogenated in an autoclave at 20-30 bar for 1.5 h. The reaction mixture was filtered through Celite, rinsed with methanol and 2 N ammonia in methanol, and concentrated. This gave 2.23 g of the target compound (94% of theory).

LC-MS (Method 6): Rₜ=1.48 min

MS (ESpos): m/z=273 (M+H)⁺

¹H-NMR (400 MHz, DMSO-d₆): δ [ppm]=1.19 (s, 3H), 1.48 (br. s, 2H), 2.08-2.40 (m, 2H), 2.53-2.72 (m, 2H; partially superposed by solvent peak), 5.00 (s, 2H), 5.90-6.23 (m, 1H), 6.95 (br. s, 1H), 7.25-7.41 (m, 5H).

### Example 22A

### ent-Benzyl (1-amino-4,4-difluoro-2-methylbutan-2-yl)carbamate (Enantiomer B)

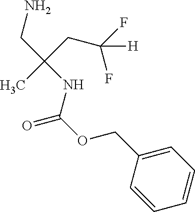 [SMILES: CC(CN)(CC(F)F)NC(=O)OCc1ccccc1]

2.76 g (10.29 mmol) of ent-benzyl (2-cyano-4,4-difluorobutan-2-yl)carbamate (enantiomer B) from Example 20A were dissolved in 90 ml of 7 N ammonia solution in methanol, and 3.19 g of Raney nickel (50% aqueous slurry) were added under argon. The reaction mixture was hydrogenated in an autoclave at 20-30 bar for 1.5 h. The reaction mixture was filtered through Celite, rinsed with methanol and 2 N ammonia in methanol, and concentrated. This gave 2.64 g of the target compound (88% of theory, purity 93%).

LC-MS (Method 6): Rₜ=1.49 min

MS (ESpos): m/z=273 (M+H)⁺

¹H-NMR (400 MHz, DMSO-d₆): δ [ppm]=1.19 (s, 3H), 1.48 (br. s, 2H), 2.08-2.40 (m, 2H), 2.53-2.73 (m, 2H; partially superposed by solvent peak)), 5.00 (s, 2H), 5.90-6.24 (m, 1H), 6.95 (br. s, 1H), 7.25-7.41 (m, 5H).

### Example 23A

### ent-Benzyl {1-[({8-[(2,6-difluorobenzyl)oxy]quinolin-4-yl}carbonyl)amino]-5,5,5-trifluoro-2-methylpentan-2-yl}carbamate trifluoroacetate (Enantiomer B)

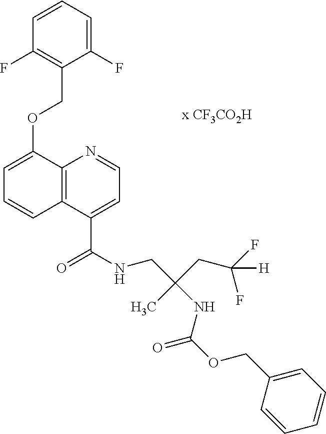 [SMILES: CC(CNC(=O)c1ccnc2c(OCc3c(F)cccc3F)cccc12)(CC(F)F)NC(=O)OCc1ccccc1]

90 mg (0.29 mmol) of 8-[(2,6-difluorobenzyl)oxy]quinoline-4-carboxylic acid from Example 4A were dissolved in 0.95 ml of DMF, 141 mg (0.37 mmol) of HATU and 0.25 ml (1.43 mmol) of N,N-diisopropylethylamine were added and the mixture was stirred at room temperature for 20 min. Subsequently, 119 mg (0.37 mmol) of ent-benzyl (1-amino-5,5,5-trifluoro-2-methylpentan-2-yl)carbamate (enantiomer B) from Example 14A were added. The mixture was stirred at RT for 30 min, water, acetonitrile and TFA were subsequently added and the reaction mixture was purified by preparative HPLC (RP18 column, mobile phase: acetonitrile/water gradient with addition of 0.1% TFA). The product fractions were combined and concentrated. 181 mg of the title compound were obtained (88% of theory).

LC-MS (Method 5): Rₜ=1.17 min

MS (ESpos): m/z=602 (M-TFA+H)⁺

### Example 24A

### ent-Benzyl {1-[({8-[(2,6-difluorobenzyl)oxy]quinolin-4-yl}carbonyl)amino]-4,4-difluoro-2-methylbutan-2-yl}carbamate trifluoroacetate (Enantiomer B)

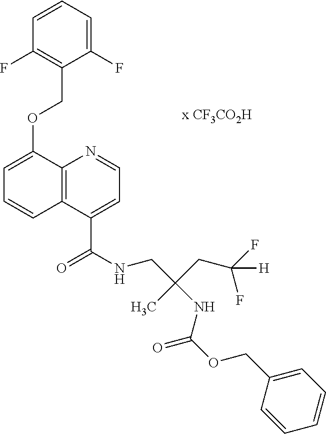 [SMILES: CC(CNC(=O)c1ccnc2c(OCc3c(F)cccc3F)cccc12)(CC(F)F)NC(=O)OCc1ccccc1]

90 mg (0.29 mmol) of 8-[(2,6-difluorobenzyl)oxy]quinoline-4-carboxylic acid from Example 4A were dissolved in 0.95 ml of DMF, 141 mg (0.37 mmol) of HATU and 0.25 ml (1.43 mmol) of N,N-diisopropylethylamine were added and the mixture was stirred at room temperature for 20 min. 108 mg (0.37 mmol, 93% purity) of ent-benzyl (1-amino-4,4-difluoro-2-methylbutan-2-yl)carbamate (enantiomer B) from Example 22A were then added. The mixture was stirred at RT for 30 min, water, acetonitrile and TFA were subsequently added and the reaction mixture was purified by preparative HPLC (RP18 column, mobile phase: acetonitrile/water gradient with addition of 0.1% TFA). The product fractions were combined and concentrated to dryness under reduced pressure. 124 mg of the title compound were obtained (62% of theory).

LC-MS (Method 5): Rₜ=1.11 min

MS (ESpos): m/z=570 (M-TFA+H)⁺

### Example 25A

### 3,3,4,4,4-Pentafluorobutyl trifluoromethanesulfonate

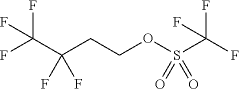 [SMILES: O=S(=O)(OCCC(F)(F)C(F)(F)F)C(F)(F)F]

198.49 g (703.51 mmol) of trifluoromethanesulfonic anhydride were initially charged under argon. The reaction flask was immersed into an oil bath at 70° C. and heated to internal temperature 56° C. 88.2 ml (738.68 mmol) of 3,3,4,4,4-pentafluorobutan-1-ol were added dropwise to the reaction mixture within 35 min and the mixture was stirred at bath temperature 70-73° C. and internal temperature 69° C. for two hours. The filtrate was concentrated on a rotary evaporator and the residue was taken up in 1500 ml of dichloromethane. The residue was washed once with 300 ml of cold water, once with 300 ml of cold saturated aqueous sodium hydrogencarbonate solution and once with 300 ml of cold water. The organic phase was dried with magnesium sulfate, filtered and concentrated. This gave 192.86 g (92.6% of theory) of the target compound.

¹H-NMR (400 MHz, DMSO-d₆): δ=2.71-2.89 (m, 2H), 4.58 (t, 2H).

### Example 26A

### rac-Methyl 5,5,6,6,6-pentafluoronorleucinate hydrochloride (Racemate)

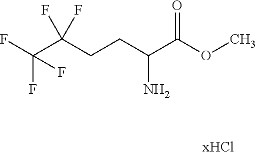

132 g (521.0 mmol) of methyl N-(diphenylmethylene)glycinate [described in: WO2010/123792 A1, 2010; p. 11-13] were initially charged in 1000 ml of THF (anhydrous) under argon and cooled to −40° C. 625.2 ml (625.20 mmol) of bis(trimethylsilyl)lithium amide (1 M in THF) were added dropwise within 30 min. After 10 min at −40° C., the internal temperature was allowed to rise to 0° C. within 35 min. 192.86 g (651.25 mmol) of 3,3,4,4,4-pentafluorobutyl trifluoromethanesulfonate from Example 25A, dissolved in 400 ml of THF, were added dropwise to the reaction solution at 0° C. After 10 min, the cooling bath was removed and the mixture was stirred at RT for 3 h. Subsequently, the reaction mixture was cooled to 0° C. and 410 ml (1.33 mol) of 3 N aqueous hydrochloric acid were added dropwise. The cooling bath was removed and the reaction solution was stirred at RT for two hours. The mixture was subsequently concentrated. This gave 141.5 g of the target compound as a crude mixture, which was used in the subsequent stage without further purification.

### Example 27A

### rac-Methyl N-[(benzyloxy)carbonyl]-5,5,6,6,6-pentafluoronorleucinate (Racemate)

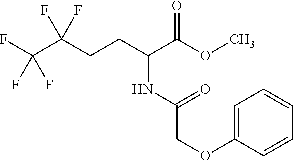 [SMILES: COC(=O)C(CCC(F)(F)C(F)(F)F)NC(=O)COc1ccccc1]

141.5 g (520.99 mmol) of rac-methyl 5,5,6,6,6-pentafluoronorleucinate hydrochloride from Example 26A were taken up in in 850 ml of THF and 850 ml of water under argon, and 223.2 g (1.62 mol) of potassium carbonate were added cautiously at RT. Subsequently, 82 ml (573.09 mmol) of benzyl chloroformate were added dropwise and the mixture was stirred at RT overnight. The reaction mixture was extracted twice with 500 ml of ethyl acetate, and the organic phase was dried with magnesium sulfate, filtered and concentrated. The residue was diluted in 50 ml of dichloromethane and purified by means of silica gel chromatography (mobile phase: cyclohexane/ethyl acetate 9/1 to 4/1). The isolated product fractions were purified once more by means of preparative HPLC [column: Daiso C18 10 μm Bio 300×100mm, neutral; mobile phase: acetonitrile/water gradient; flow rate: 250 ml/min; temperature: RT; wavelength: 210 nm]. This gave 27.4 g (14% of theory) of the target compound.

LC-MS (Method 5): Rₜ=1.09 min.

MS (ESIpos): m/z=370 (M+H)⁺.

¹H-NMR (400 MHz, DMSO-d₆): δ=1.78-1.91 (m, 1H), 1.93-2.05 (m, 1H), 2.10-2.30 (m, 1H), 2.30-2.46 (m, 1H), 3.66 (s, 3H), 4.18-4.26 (m, 1H), 5.05 (s, 2H), 7.27-7.40 (m, 5H), 7.89 (d, 1H).

### Example 28A

### rac-Benzyl (6,6,7,7,7-pentafluoro-2-hydroxy-2-methylheptan-3-yl)carbamate (Racemate)

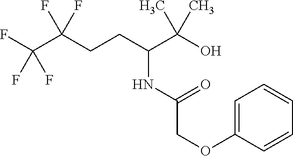 [SMILES: CC(C)(O)C(CCC(F)(F)C(F)(F)F)NC(=O)COc1ccccc1]

1.7 g (3.68 mmol, purity 80%) of rac-methyl N-[(benzyloxy)carbonyl]-5,5,6,6,6-pentafluoronorleucinate (racemate) from Example 27A were initially charged in THF under argon and the reaction mixture was cooled to 0° C. 4.3 ml (12.89 mmol) of 3M methylmagnesium bromide in diethyl ether were added dropwise and the mixture was stirred at 0° C. for 15 min. The mixture was then allowed to warm up gradually to RT and stirred at room temperature overnight. Saturated aqueous ammonium chloride solution was added cautiously to the reaction mixture and then the reaction solution was concentrated to half its volume. The residue was partitioned between dichloromethane and water, and the organic phase was washed twice with water, dried over sodium sulfate, filtered and concentrated. The residue was purified by means of silica gel chromatography (cyclohexane/ethyl acetate 10:1 to 7:3). This gave 1.31 g (96% of theory) of the target compound.

LC-MS (Method 5): Rₜ=1.03 min.

MS (ESIpos): m/z=370 (M+H)⁺.

¹H-NMR (400 MHz, DMSO-d₆): δ=1.01 (s, 3H), 1.08 (s, 3H), 1.43-1.56 (m, 1H), 1.92-2.01 (m, 1H), 2.01-2.19 (m, 2H), 3.36-3.44 (m, 1H), 4.48 (s, 1H), 4.99-5.12 (m, 2H), 7.11 (d, 1H), 7.27-7.38 (m, 5H).

### Example 29A

### ent-Benzyl (6,6,7,7,7-pentafluoro-2-hydroxy-2-methylheptan-3-yl)carbamate (Enantiomer A)

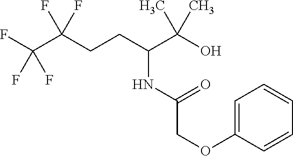 [SMILES: CC(C)(O)C(CCC(F)(F)C(F)(F)F)NC(=O)COc1ccccc1]

1.31 g of Example 28A were separated into the enantiomers by preparative separation on a chiral phase [column: Daicel Chiralpak AY-H, 5 μm, 250×20 mm, mobile phase: 90% isohexane, 10% ethanol, flow rate: 15 ml/min; 35° C.; detection: 220 nm].

Enantiomer A:

yield: 459 mg (99% ee)

Rₜ=4.31 min [Daicel Chiralpak AY-H, 5 μm, 250×4.6 mm; mobile phase: 90% isohexane, 10% ethanol; flow rate: 1.0 ml/min; 30° C.; detection: 220 nm].

### Example 30A

### ent-3-Amino-6,6,7,7,7-pentafluoro-2-methylheptan-2-ol hydrochloride (Enantiomer A)

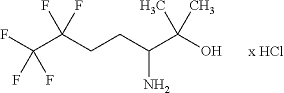

To an initial charge of 455 mg (1.23 mmol) of ent-benzyl (6,6,7,7,7-pentafluoro-2-hydroxy-2-methylheptan-3-yl)carbamate (enantiomer A) from Example 29A in 8.6 ml of ethanol were added 131 mg of palladium on charcoal (10%) and 3.74 ml (36.96 mmol) of cyclohexene, and the mixture was stirred under reflux for 3 h. The reaction mixture was filtered through a Millipore filter and washed with ethanol. 1.23 ml of hydrogen chloride (2 N in diethyl ether) were added to the filtrate and the mixture was concentrated and dried under high vacuum. This gave 335 mg (98% of theory) of the target compound.

MS (Method 10): m/z=236 (M-HCl+H)⁺

¹H-NMR (400 MHz, DMSO-d₆): δ=1.11 (s, 3H), 1.22 (s, 3H), 1.58-1.72 (m, 1H), 1.80-1.92 (m, 1H), 2.27-2.46 (m, 2H, partially obscured by DMSO peak), 2.94-3.04 (m, 1H), 5.35 (s, 1H), 7.80-8.01 (m, 3H).

### Example 31A

### Ethyl 8-[(2,6-difluorobenzyl)oxy]quinoline-3-carboxylate

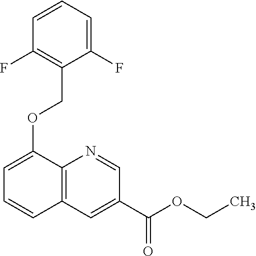 [SMILES: CCOC(=O)c1cnc2c(OCc3c(F)cccc3F)cccc2c1]

250 mg (1.15 mmol) of ethyl 8-hydroxyquinoline-3-carboxylate were initially charged in 16.5 ml of abs. DMF, and 825 mg (2.53 mmol) of cesium carbonate and 262 mg (1.27 mmol) of 2,6-difluorobenzyl bromide were added. The mixture was stirred at RT for 1 h. The reaction mixture was poured onto about 130 ml of water and stirred for 30 min. The solid formed was filtered off and dried under high vacuum. This gave 393 mg (99% of theory) of the target compound.

LC-MS (Method 5): Rₜ=1.13 min.

MS (ESIpos): m/z=344 (M+H)⁺.

¹H-NMR (500 MHz, DMSO-d₆): δ [ppm]=1.38 (t, 3H), 4.41 (q, 2H), 5.34 (s, 2H), 7.20-7.27 (m, 2H), 7.55-7.62 (m, 2H), 7.68 (t, 1H), 7.81 (d, 1H), 8.96 (d, 1H), 9.23 (d, 1H).

### Example 32A

### 8-[(2,6-Difluorobenzyl)oxy]quinoline-3-carboxylic acid

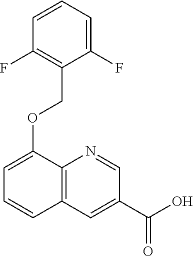 [SMILES: O=C(O)c1cnc2c(OCc3c(F)cccc3F)cccc2c1]

393 mg (1.14 mmol) of ethyl-8-[(2,6-difluorobenzyl)oxy]quinoline-3-carboxylate from Example 31A were dissolved in 24.5 ml of THF/methanol (5/1), 5.74 ml (5.74 mmol) of a 1 N lithium hydroxide solution were added and the mixture was stirred at 60° C. overnight. Another 6 ml of THF/methanol (5/1) and 5.74 ml (5.74 mmol) of a 1 N lithium hydroxide solution were then added, and the mixture was stirred under reflux for 6 h. The mixture was then concentrated by evaporation, and 25 ml of dioxane were added. After addition of 5.74 ml (5.74 mmol) of 1 N aqueous sodium hydroxide solution, the mixture was stirred under reflux for 4 h. The reaction solution was concentrated by evaporation. The residue was taken up in a little THF and then acidified with 1 N aqueous hydrochloric acid. A little THF was evaporated. The solid formed was filtered off, washed with water and dried under high vacuum. This gave 379 mg (99% of theory, purity 94%) of the target compound.

LC-MS (Method 5): Rₜ=0.84 min.

MS (ESIpos): m/z=316 (M+H)⁺.

¹H-NMR (500 MHz, DMSO-d₆): δ [ppm]=5.33 (s, 2H), 7.18-7.26 (m, 2H), 7.54-7.62 (m, 2H), 7.67 (t, 1H), 7.80 (d, 1H), 8.97 (d, 1H), 9.22 (d, 111), 13.56 (br. s, 1H).

### Example 33A

### ent-Benzyl {1-[({8-[(2,6-difluorobenzyl)oxy]quinolin-3-yl}carbonyl)amino]-5,5,5-trifluoro-2-methylpentan-2-yl carbamate trifluoroacetate (Enantiomer B)

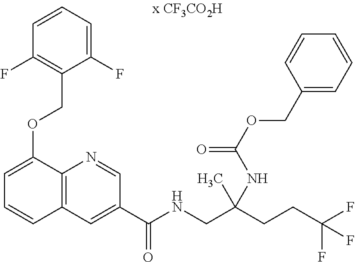 [SMILES: CC(CCC(F)(F)F)(CNC(=O)c1cnc2c(OCc3c(F)cccc3F)cccc2c1)NC(=O)OCc1ccccc1]

The target compound was obtained by reacting 8-[(2,6-difluorobenzyl)oxy]quinoline-3-carboxylic acid from Example 32A analogously to Example 23A with ent-benzyl (1-amino-5,5,5-trifluoro-2-methylpentan-2-yl)carbamate (Enantiomer B) from Example 14A [yield: 60% of theory].

LC-MS (Method 5): Rₜ=1.21 min

MS (ESpos): m/z=602 (M-TFA+H)⁺

### Example 34A

### Benzyl {(2S)-1-[({8- [(2,6-difluorobenzyl)oxy]quinolin-3-yl}carbonyl)amino]-2-methylbutan-2-yl}carbamate trifluoroacetate

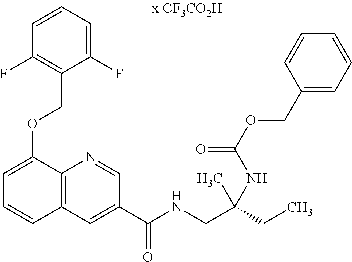 [SMILES: CC[C@@](C)(CNC(=O)c1cnc2c(OCc3c(F)cccc3F)cccc2c1)NC(=O)OCc1ccccc1]

The target compound was obtained by reacting 8-[(2,6-difluorobenzyl)oxy]quinoline-3-carboxylic acid from Example 32A analogously to Example 23A with benzyl [(2S)-1-amino-2-methylbutan-2-yl)carbamate from Example 5A [yield: 50% of theory].

LC-MS (Method 5): Rₜ=1.16 min

MS (ESpos): m/z=534 (M-TFA+H)⁺

### Example 35A

### 4-Bromo-6-methylquinolin-8-ol

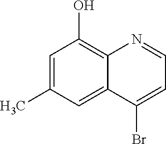

9.92 g (39.35 mmol) of 4-bromo-8-methoxy-6-methylquinoline were dissolved in 496 ml 48% strength aqueous hydrobromic acid (CAS No.: 10035-10-6). The reaction mixture was stirred under reflux overnight. The reaction mixture was then concentrated and co-distilled twice with toluene. The residue was dried under high vacuum and reacted further, assuming a yield of 100%.

LC-MS (Method 5): Rₜ=0.97 min

MS (ESpos): m/z=238 (M+H)⁺

### Example 36A

### 4-Bromo-8-[(2,6-difluorobenzyl)oxy]-6-methylquinoline

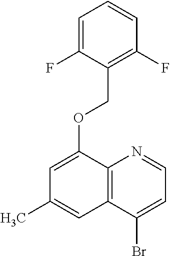 [SMILES: Cc1cc(OCc2c(F)cccc2F)c2nccc(Br)c2c1]

9.37 g (39.36 mmol) of 4-bromo-6-methylquinolin-8-ol from Example 35A were initially charged in 100 ml of N,N′-dimethylformamide, and 10.59 g (51.16 mmol) of 2,6-difluorobenzyl bromide and 38.47 g (118.1 mmol) of cesium carbonate were added. The reaction mixture was stirred at 60° C. for 2 hours and then cooled. 200 ml of water and a little ice were added and the mixture was stirred for 5 minutes. The resulting solid was filtered off with suction, washed repeatedly with water and then repeatedly stirred with acetonitrile and filtered off with suction again. This gave, after drying under high vacuum, 12.8 g (89% of theory) of the title compound.

LC-MS (Method 5): Rₜ=1.20 min

MS (ESpos): m/z=366/364 (M+H)⁺

¹H-NMR (400 MHz, DMSO-d₆): δ [ppm]=2.56 (s, 3H), 5.31 (s, 2H), 7.19-7.28 (m, 2H), 7.40 (s, 1H), 7.53 (s, 1H), 7.55-7.64 (m, 1H), 7.90 (d, 1H), 8.54 (d, 1H).

### Example 37A

### 8-[(2,6-Difluorobenzyl)oxy]-6-methylquinoline-4-carbonitrile

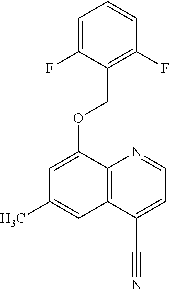 [SMILES: Cc1cc(OCc2c(F)cccc2F)c2nccc(C#N)c2c1]

12.8 g (35.15 mmol) of 4-bromo-8-[(2,6-difluorobenzyl)oxy]-6-methylquinoline from Example 36A and 15.74 g (175.7 mmol) of copper(I) cyanide (CAS No.: 544-92-3) were dissolved in 150 ml of dimethyl sulfoxide, and the mixture was stirred at 160° C. for 2 hours. The reaction mixture was then cooled, tert-butyl methyl ether and water were added and the mixture was stirred and filtered off with suction over kieselguhr. The phases of the filtrate were separated, and the aqueous phase was extracted twice with tert-butyl methyl ether. The combined organic phases were washed with saturated aqueous sodium bicarbonate solution, dried over sodium sulfate and concentrated. The precipitate, which was filtered off with suction through kieselguhr, was stirred in dichloromethane/methanol (10/1), filtered through Celite and washed twice with dichloromethane/methanol (10/1). The filtrate was washed with saturated aqueous sodium bicarbonate solution, combined with the solid isolated first, and concentrated. The residue was purified by silica gel chromatography (mobile phase: cyclohexane/ethyl acetate: 4/1, 2/1, 1/1). This gave 3.65 g (33% of theory) of the title compound.

LC-MS (Method 5): Rₜ=1.10 min

MS (ESpos): m/z=311 (M+H)⁺

¹H-NMR (400 MHz, DMSO-d₆): δ [ppm]=2.60 (s, 3H), 5.34 (s, 2H), 7.19-7.28 (m, 2H), 7.50 (s, 2H), 7.54-7.64 (m, 1H), 8.09 (d, 1H), 8.91 (d, 1H).

### Example 38A

### 8-[(2,6-Difluorobenzyl)oxy]-6-methylquinoline-4-carboxylic acid

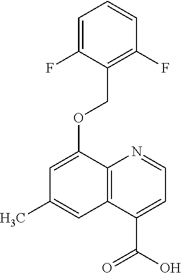 [SMILES: Cc1cc(OCc2c(F)cccc2F)c2nccc(C(=O)O)c2c1]

3.65 g (11.76 mmol) of 8-[(2,6-difluorobenzyl)oxy]-6-methylquinoline-4-carbonitrile from Example 37A were dissolved in 100 ml of ethanol, 118 ml (235.25 mmol) of a 2N aqueous sodium hydroxide solution were added and the mixture was heated under reflux overnight. The reaction mixture was then cooled, diluted with water and adjusted to pH=2 using aqueous 1N hydrochloric acid. Ice was added until the internal temperature was 10° C., and the mixture was stirred for another 1 hour. The resulting solid was filtered off with suction and washed repeatedly with water. 3.50 g (90% of theory) of the title compound were obtained.

LC-MS (Method 5): Rₜ=0.68 min

MS (ESpos): m/z=330 (M+H)⁺

¹H-NMR (400 MHz, DMSO-d₆): δ [ppm]=5.31 (s, 2H), 7.19-7.28 (m, 2H), 7.35 (s, 1H), 7.53-7.63 (m, 1H), 7.86 (d, 1H), 8.03 (s, 1H) 8.84 (d, 1H), 13.78 (s, 1H), [further signal under solvent peak].

### Example 39A

### ent-Benzyl {1-[({8-[(2,6-difluorobenzyl)oxy]-6-methylquinolin-4-yl}carbonyl)amino]-5,5,5-trifluoro-2-methylpentan-2-yl}carbamate (Enantiomer B)

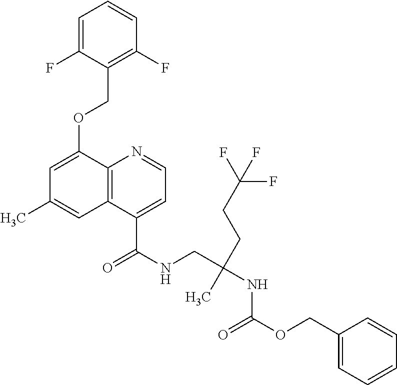 [SMILES: Cc1cc(OCc2c(F)cccc2F)c2nccc(C(=O)NCC(C)(CCC(F)(F)F)NC(=O)OCc3ccccc3)c2c1]

80 mg (0.24 mmol) of 8-[(2,6-difluorobenzyl)oxy]-6-methylquinoline-4-carboxylic acid from Example 38A were dissolved in 1.62 ml of DMF, 120 mg (0.32 mmol) of HATU and 0.21 ml (1.22 mmol) of N,N-diisopropylethylamine were added and the mixture was stirred at room temperature for 20 min. 109 mg (0.32 mmol, purity 88%) of ent-benzyl (1-amino-5,5,5-trifluoro-2-methylpentan-2-yl)carbamate (Enantiomer B) from Example 14A were then added, and the mixture was stirred at RT for one hour. Water was added, and the reaction mixture was stirred at RT for 25 minutes. The solid formed was filtered off with suction, washed with water and dried under high vacuum. This gave 160 mg of the title compound (100% of theory; purity 94%).

LC-MS (Method 5): Rₜ=1.23 min

MS (ESpos): m/z=616.5 (M+H)⁺

### Example 40A

### 4-Bromo-2-methylquinolin-8-ol

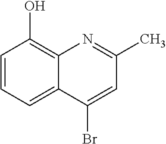

A solution of 23 g (90.2 mmol) of 4-bromo-8-methoxy-2-methylquinoline in 230 ml of a 48% strength solution of hydrobromic acid in water was heated at reflux overnight. The mixture was allowed to cool to RT and the pH was adjusted to 7 by addition of a 2 M solution of sodium hydroxide. The resulting mixture was extracted with dichloromethane, the organic phase was dried over anhydrous sodium sulfate and the solvent was removed under reduced pressure. This gave 16.8 g of the target product (21% of theory).

¹H-NMR (300 MHz, CDCl₃): δ [ppm]=7.65-7.56 (m, 1H), 7.51-7.40 (m, 2H), 7.22-7.16 (m, 1H), 2.69 (s, 3H).

### Example 41A

### 4-Bromo-8((2,6-difluorobenzyl)oxy)-2-methylquinoline

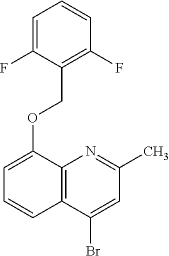 [SMILES: Cc1cc(Br)c2cccc(OCc3c(F)cccc3F)c2n1]

22.9 g (70.4 mmol) of cesium carbonate were added to a stirred solution of 16.8 g (70.4 mmol) of 4-bromo-2-methylquinolin-8-ol from Example 40A and 17.5 g (80.5 mmol) of 2-(bromomethyl)-1,3-difluorobenzene, and the resulting mixture was stirred at RT for two days. The mixture was concentrated under reduced pressure and partitioned between dichloromethane and water, and the organic phase was washed with water. The phases were separated, the organic phase was dried over anhydrous sodium sulfate and the solvent was removed under reduced pressure. This gave 23.8 g of the target product (92% of theory).

¹H-NMR (300 MHz, CDCl₃): δ [ppm]=7.80 (d, 1H), 7.62 (s, 1H), 7.48 (t, 1H), 7.38-7.24 (m, 2H), 5.46 (s, 2H), 2.71 (s, 3H).

### Example 42A

### 8-((2,6-Difluorobenzyl)oxy)-2-methylquinoline-4-carbonitrile

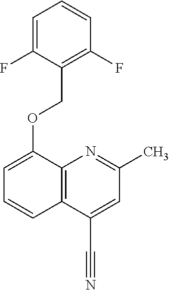 [SMILES: Cc1cc(C#N)c2cccc(OCc3c(F)cccc3F)c2n1]

In the microwave, a solution of 22.7 g (62.3 mmol) of 4-bromo-8-((2,6-difluorobenzyl)oxy)-2-methylquinoline from Example 41A and 22.0 g (324.1 mmol) of copper(I) cyanide in 450 ml of dry DMSO was heated at 160° C. for 60 minutes. The solvent was removed under reduced pressure and the residue was purified by chromatography on silica gel (mobile phase: heptane/ethyl acetate 50:50). This gave 2.5 g of the target product (13% of theory).

¹H-NMR (300 MHz, CDCl₃): δ [ppm]=7.78 (d, 1H), 7.63-7.58 (m, 2H), 7.39-7.25 (m, 2H), 7.00-6.88 (m, 2H), 7.49 (s, 2H), 5.44 (s, 2H), 2.81 (s, 3H).

### Example 43A

### 8((2,6-Difluorobenzyl)oxy)-2-methylquinoline-4-carboxylic acid

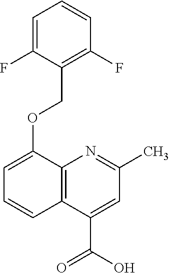 [SMILES: Cc1cc(C(=O)O)c2cccc(OCc3c(F)cccc3F)c2n1]

1.8 g (22.6 mmol) of sodium peroxide were added to a solution of 1.4 g (4.5 mmol) of 84(2,6-difluorobenzyl)oxy)-2-methylquinoline-4-carbonitrile from Example 42A in 30 ml of water, and the resulting suspension was heated at reflux for two days. The mixture was allowed to cool to RT and filtered through Celite. The filtrate was diluted with water and adjusted to a pH of 5 using a 1 M solution of hydrochloric acid. The aqueous phase was extracted with ethyl acetate, the phases were separated, the organic phase was dried over anhydrous sodium sulfate and the solvent was removed under reduced pressure. This gave 503 mg of the target product (29% of theory).

LC-MS (Method 14): Rₜ=2.18 min; m/z=330 (M+H)⁺

¹H-NMR (300 MHz, DMSO-d₆): δ [ppm]=8.18 (d, 1H), 7.80 (s, 1H), 7.61-7.52 (m, 2H), 7.42 (d, 1H), 7.21 (t, 2H), 5.36 (s, 2H), 2.63 (s, 3H).

### Example 44A

### ent-Benzyl {1-[({8-[(2,6-difluorobenzyl)oxy]-2-methylquinolin-4-yl} carbonyl)amino]-5,5,5-trifluoro-2-methylpentan-2-yl}carbamate trifluoroacetate (Enantiomer B)

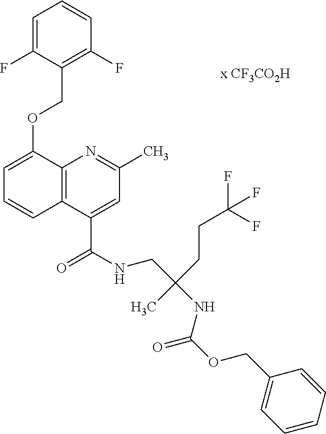 [SMILES: Cc1cc(C(=O)NCC(C)(CCC(F)(F)F)NC(=O)OCc2ccccc2)c2cccc(OCc3c(F)cccc3F)c2n1]

50 mg (0.13 mmol, purity 85%) of 8-[(2,6-difluorobenzyl)oxy]-2-methylquinoline-4-carboxylic acid from Example 43A were dissolved in 0.43 ml of DMF, 64 mg (0.17 mmol) of HATU and 0.11 ml (0.65 mmol) of N,N-diisopropylethylamine were added and the mixture was stirred at room temperature for 20 min. 58 mg (0.17 mmol, purity about 95%) of ent-benzyl (1-amino-5,5,5-trifluoro-2-methylpentan-2-yl)carbamate (Enantiomer B) from Example 14A were then added, and the mixture was stirred at RT for 30 minutes. Acetonitrile, water and TFA were added and the reaction solution was purified by preparative HPLC (RP18 column, mobile phase: acetonitrile/water gradient with addition of 0.1% TFA). This gave 60 mg of the title compound (62% of theory; purity 97%).

LC-MS (Method 5): Rₜ=1.16 min

MS (ESpos): m/z=616 (M-TFA+H)⁺

### Example 45A

### ent-Benzyl {1-[({8-[(2,6-difluorobenzyl)oxy]-2-methylquinolin-4-yl}carbonyl)amino]-2-methylbutan-2-yl}carbamate trifluoroacetate (Enantiomer B)

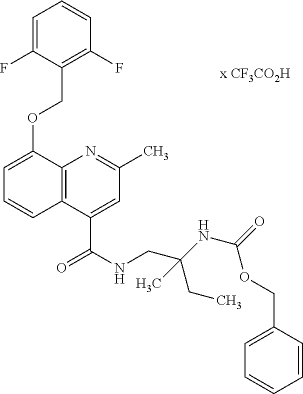 [SMILES: CCC(C)(CNC(=O)c1cc(C)nc2c(OCc3c(F)cccc3F)cccc12)NC(=O)OCc1ccccc1]

60 mg (0.18 mmol) of 8-[(2,6-difluorobenzyl)oxy]-2-methylquinoline-4-carboxylic acid from Example 43A were dissolved in 0.62 ml of DMF, 90 mg (0.24 mmol) of HATU and 0.16 ml (0.91 mmol) of N,N-diisopropylethylamine were added and the mixture was stirred at room temperature for 20 min 56 mg (0.24 mmol) of ent-benzyl (1-amino-2-methylbutan-2-yl)carbamate (Enantiomer B) from Example 5A were then added, and the mixture was stirred at RT for 3 days. Water and ethyl acetate were added to the reaction solution. The aqueous phase was extracted three times with ethyl acetate, and the combined organic phases were dried over sodium sulfate, filtered and concentrated. Acetonitrile, water and TFA were added to the residue and the product was purified by preparative HPLC (RP18 column, mobile phase: acetonitrile/water gradient with addition of 0.1% TFA). This gave 54 mg of the title compound (43% of theory; purity 96%).

LC-MS (Method 5): Rₜ=1.13 min

MS (ESpos): m/z=548 (M-TFA+H)⁺

### Example 46A

### rac-2-Amino-2-[2-(difluoromethyl)-2H-tetrazol-5-yl] propan-1-ol

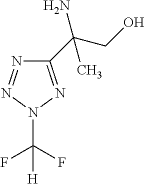

The target compound can be prepared by deprotection of 1-{[tert-butyl(dimethyl)silyl]oxy}-2-[2-(difluoromethyl)-2H-tetrazol-5-yl]propane-2-amine (preparable analogously to intermediate 300 in WO2014/084312 from racemic starting material) using tetrabutylammonium fluoride (TBAF) in THF at room temperature, according to methods known from the literature.

### Working Examples

### Example 1

### 8-[(2,6-Difluorobenzyl)oxy]-N-[1-(3,4-difluorophenyl)cyclopropyl]quinoline-4-carboxamide

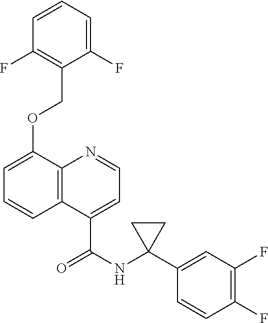 [SMILES: O=C(NC1(c2ccc(F)c(F)c2)CC1)c1ccnc2c(OCc3c(F)cccc3F)cccc12]

68 mg (0.357 mmol) of 1-(3-dimethylaminopropyl)-3-ethylcarbodiimide hydrochloride and 55 mg (0.358 mmol) of 1-hydroxybenzotriazole were added to a solution of 75 mg (0.238 mmol) of 8-[(2,6-difluorobenzyl)oxy]quinoline-4-carboxylic acid (Example 4A) in 14 ml of dichloromethane, and the mixture was stirred at room temperature for 15 minutes. 80 mg (0.476 mmol) of 1-(3,4-difluorophenyl)cyclopropylamine hydrochloride (CAS No.: 1186663-16-0) and 0.207 ml of N,N-diisopropylethylamine were added to the reaction mixture. The mixture was stirred at room temperature for a further 18 hours. The reaction mixture was concentrated under reduced pressure and the residue was partitioned between water and dichloromethane. The aqueous phase was extracted twice with dichloromethane and the combined organic extracts were washed with saturated aqueous sodium bicarbonate solution, dried over magnesium carbonate and concentrated under reduced pressure. The residue was purified by preparative HPLC chromatography (Method 4), which gave 37 mg (33% of theory) of the target compound.

LC-MS (Method 3): Rₜ=12.28 min; m/z=467.25 (M+H)⁺

¹H-NMR (600 MHz, DMSO-dₒ): δ [ppm]=1.31-1.36 (m, 4H), 5.32 (s, 2H), 7.15-7.19 (m, 1H), 7.20-7.24 (m, 2H), 7.29 (ddd, 1H), 7.39 (dt, 1H), 7.43 (dd, 1H), 7.53-7.62 (m, 3H), 7.63 (d, 1H), 8.88 (d, 1H), 9.50 (s, 1H).

### Example 2

### 8-[(2,6-Difluorobenzyl)oxy]-N-(6-fluoroquinolin-4-yl)quinoline-4-carboxamide

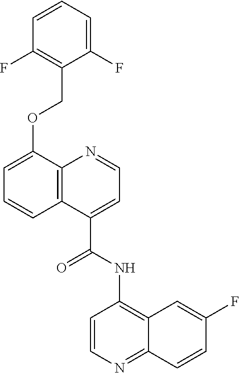 [SMILES: O=C(Nc1ccnc2ccc(F)cc12)c1ccnc2c(OCc3c(F)cccc3F)cccc12]

41 mg (0.254 mmol) of 1,1¹-carbonyldiimidazole and 21 mg (0.137 mmol) of 1-hydroxybenzotriazole were added to a solution of 62 mg (0.195 mmol) of 84(2,6-difluorobenzyl)oxy]quinoline-4-carboxylic acid (Example 4A) in 2 ml of N,N′-dimethylformamide. The mixture was stirred at room temperature for a further 15 minutes. 41 mg (0.254 mmol) of 6-fluoro-4-quinolineamine (CAS No.: 874800-60-9) and 0.068 ml of N,N-diisopropylethylamine were added to the reaction mixture. The reaction mixture was heated in a microwave at 100° C. for 20 minutes and then allowed to cool and concentrated under reduced pressure. The residue was partitioned between water and dichloromethane and the aqueous phase was extracted twice with dichloromethane. The combined organic phases were washed with saturated aqueous sodium bicarbonate solution, dried over sodium sulfate and concentrated under reduced pressure. The residue was purified by preparative HPLC chromatography (Method 4), which gave 17 mg (18% of theory) of the target compound.

LC-MS (Method 1): Rₜ=1.10 min; m/z=460.23 (M+H)⁺

¹H-NMR (600 MHz, DMSO-d₆): δ [ppm]=5.36 (s, 2H), 7.20-7.26 (m, 2H), 7.49 (dd, 1H), 7.58 (tt, 1H), 7.62-7.66 (m, 1H), 7.71 (ddd, 1H), 7.75 (dd, 1H), 7.90 (d, 1H), 8.09-8.16 (m, 2H), 8.29 (br. s., 1H), 8.92 (d, 1H), 8.99 (d, 1H), 11.01 (s, 1H).

### Example 3

### 8-[(2,6-Difluorobenzyl)oxy]-N-[(2S)-hexan-2-yl]quinoline-4-carboxamide

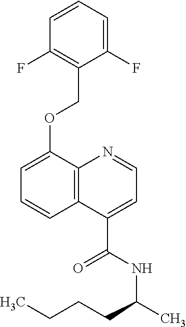 [SMILES: CCCC[C@H](C)NC(=O)c1ccnc2c(OCc3c(F)cccc3F)cccc12]

41 mg (0.254 mmol) of 1,1′-carbonyldiimidazole and 21 mg (0.137 mmol) of 1-hydroxybenzotriazole were added to a solution of 62 mg (0.195 mmol) of 8-[(2,6-difluorobenzyl)oxy]quinoline-4-carboxylic acid (Example 4A) in 2 ml of N,N′-dimethylformamide, and the mixture was stirred at room temperature for 15 minutes. 24 mg (0.234 mmol) of (S)-(+)-2-aminohexane (CAS No.:70492-67-0) and 0.068 ml of N,N-diisopropylethylamine were added. The reaction mixture was heated in a microwave at 100° C. for 15 minutes, allowed to cool and concentrated under reduced pressure. The residue was partitioned between water and dichloromethane and the aqueous phase was extracted twice with dichloromethane. The combined organic phases were washed with saturated aqueous sodium bicarbonate solution, dried over sodium sulfate and concentrated under reduced pressure. The residue was purified by preparative HPLC chromatography (Method 4), which gave 43 mg (55% of theory) of the target compound.

LC-MS (Method 3): Rₜ=12.11 min; m/z=399.38 (M+H)⁺

¹H-NMR (600 MHz, DMSO-d₆): δ [ppm]=0.82-0.96 (m, 3H), 1.17 (d, J=6.6 Hz, 3H), 1.24-1.39 (m, 4H), 1.42-1.57 (m, 2H), 3.99-4.10 (m, 1H), 5.32 (s, 2H), 7.17-7.26 (m, 2H), 7.42 (d, J=7.7 Hz, 1H), 7.47 (d, J=4.2 Hz, 1H), 7.52-7.60 (m, 2H), 7.63-7.67 (m, 1H), 8.53 (d, J=8.4 Hz, 1H), 8.85 (d, J=4.2 Hz, 1H).

### Example 4

### 8-[(2,6-Difluorobenzyl)oxy]-N-[(2R)-1-hydroxyhexan-2-yl]quinoline-4-carboxamide

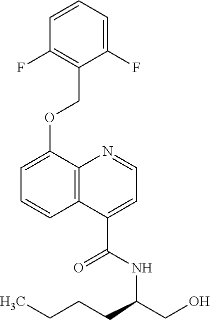 [SMILES: CCCC[C@H](CO)NC(=O)c1ccnc2c(OCc3c(F)cccc3F)cccc12]

41 mg (0.254 mmol) of 1,1′-carbonyldiimidazole and 21 mg (0.137 mmol) of 1-hydroxybenzotriazole were added to a solution of 62 mg (0.195 mmol) of 84(2,6-difluorobenzyl)oxy]quinoline-4-carboxylic acid (Example 4A) in 2 ml of N,N′-dimethylformamide, and the mixture was stirred at room temperature for 15 minutes. 29 mg (0.234 mmol) of (R)-2-amino-1-hexanol (CAS No.: 80696-28-2) and 0.068 ml of N,N-diisopropylethylamine were added to the reaction mixture. The reaction mixture was heated in a microwave at 100° C. for 20 minutes, allowed to cool and concentrated under reduced pressure. The residue was partitioned between water and dichloromethane and the aqueous phase was extracted twice with dichloromethane. The combined organic phases were washed with saturated aqueous sodium bicarbonate solution, dried over sodium sulfate and concentrated under reduced pressure. The residue was purified by preparative HPLC chromatography (Method 4), which gave 49 mg (60% of theory) of the target compound.

LC-MS (Method 3): Rₜ=10.54 min; m/z=415.29 (M+H)⁺

¹H-NMR (500 MHz, DMSO-d₆): δ [ppm]=0.90 (t, J=6.7 Hz, 3H), 1.25-1.48 (m, 5H), 1.56-1.69 (m, 1 H), 3.38-3.46 (m, 1H), 3.45-3.52 (m, 1H), 3.95-4.07 (m, 1H), 4.77 (t, J=5.6 Hz, 1H), 5.33 (s, 2H), 7.14-7.29 (m, 2H), 7.43 (d, J=7.3 Hz, 1H), 7.51 (d, J=4.0 Hz, 1H), 7.54-7.63 (m, 2H), 7.70 (dd, J=8.4, 0.8 Hz, 1H), 8.41 (d, J=8.9 Hz, 1H), 8.86 (d, J=4.3 Hz, 1H).

### Example 5

### N-[(2S)-2-Amino-2-methylbutyl]-8-[(2,6-difluorobenzyl)oxy]quinoline-4-carboxamide

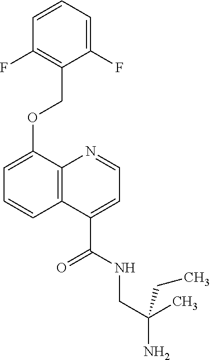 [SMILES: CC[C@](C)(N)CNC(=O)c1ccnc2c(OCc3c(F)cccc3F)cccc12]

51 mg (0.096 mmol) of benzyl {(2S)-1-[({8-(2,6-difluorobenzyl)oxy]quinolin-4-yl}carbonyl)amino]-2-methylbutan2-yl}carbcarbamate (Example 6A) were dissolved in 2 ml of dichloromethane. The reaction mixture was cooled to 0° C., and 0.096 ml of boron tribromide (1M solution in dichloromethane) was added. The mixture was stirred at room temperature for 1 hour, cooled to 0° C., and 0.143 ml of boron tribromide (1M solution in dichloromethane) was added. The reaction mixture was stirred at room temperature for 1.5 h, and 10 ml of water were then added. After the separation, the organic phase was concentrated under reduced pressure, and the residue was purified by preparative HPLC-MS chromatography (Method 4), which gave 11 mg (28% of theory) of the target compound.

LC-MS (Method 3): Rₜ=7.69 min; m/z=400.16 (M+H)⁺

¹H-NMR (500 MHz, DMSO-d₆): δ [ppm]=0.88 (t, 3H), 0.99 (s, 3H), 1.36 (qd, 2H), 1.54 (br. s., 2H), 3.25 (d, 2H), 5.32 (s, 2H), 7.23 (t, 2H), 7.44 (d, 1H),7.53-7.62 (m, 3H), 7.68 (d, 1H), 8.56 (t, 1H), 8.86 (d, 1H).

### Example 6

### ent-N-(2-Amino-5,5,5-trifluoro-2-methylpentyl)-8-[(2,6-difluorobenzyl)oxy[quinoline-4-carboxamide (Enantiomer B)

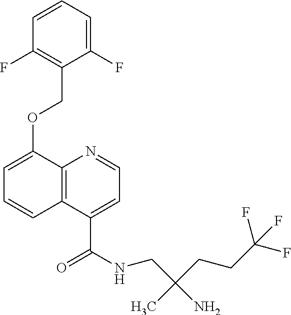 [SMILES: CC(N)(CCC(F)(F)F)CNC(=O)c1ccnc2c(OCc3c(F)cccc3F)cccc12]

181 mg (0.25 mmol) of ent-benzyl {1-[({8-[(2,6-difluorobenzyl)oxy]quinolin-4-yl}carbonyl)amino]-5,5,5-trifluoro-2-methylpentan-2-yl}carbamate trifluoroacetate (Enantiomer B) from Example 23A were dissolved in 6.4 ml of ethanol, 8 mg of palladium on activated carbon (10%) were added and the mixture was hydrogenated at atmospheric pressure for 3 hours. The reaction solution was filtered through a Millipore filter, the filter cake was washed with ethanol and the filtrate was concentrated. The residue was purified by preparative HPLC (RP18 column, mobile phase: acetonitrile/water gradient with addition of 0.1% TFA). The product fractions were combined and concentrated. Subsequently, the residue was taken up in dichloromethane and a little methanol, and washed twice with a little saturated aqueous sodium bicarbonate solution. The aqueous phase was re-extracted twice with dichloromethane. The combined organic phases were dried over sodium sulfate, filtered, concentrated and lyophilized. This gave 91 mg of the target compound (77% of theory).

LC-MS (Method 5): Rₜ=0.74 min

MS (ESpos): m/z=468 (M+H)⁺

¹H-NMR (400 MHz, DMSO-d₆): δ [ppm]=1.04 (s, 3H), 1.48-1.64 (m, 4H), 2.26-2.48 (m, 2H), 3.24-3.31 (m, 2H, partially superposed by solvent peak), 5.32 (s, 2H), 7.18-7.26 (m, 2H), 7.42 (d, 1H), 7.53-7.62 (m, 3H), 7.68 (d, 1H), 8,67 (t, 1H), 8.88 (d, 1H).

### Example 7

### ent-N-(2-Amino-4,4-difluoro-2-methylbutyl)-8-[(2,6-difluorobenzyl)oxy]quinoline-4-carboxamide (Enantiomer B)

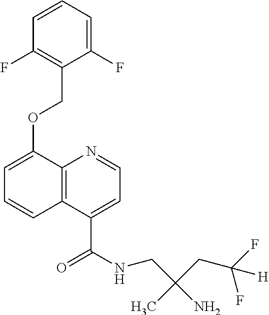 [SMILES: CC(N)(CNC(=O)c1ccnc2c(OCc3c(F)cccc3F)cccc12)CC(F)F]

124 mg (0.18 mmol) of ent-benzyl {1-[({8-[(2,6-difluorobenzyl)oxy[quinolin-4-yl}carbonyl)amino]-4,4-difluoro-2-methylbutan-2-yl}carbamate trifluoroacetate (Enantiomer B) from Example 24A were dissolved in 4.6 ml of ethanol, 6 mg of palladium on activated carbon (10%) were added and the mixture was hydrogenated at atmospheric pressure for 5 h. The reaction solution was filtered through a Millipore filter and the filtrate was concentrated. The residue was purified by preparative HPLC (RP18 column, mobile phase: acetonitrile/water gradient with addition of 0.1% TFA). The product fractions were combined and concentrated. Subsequently, the residue was taken up in dichloromethane and a little methanol, and washed twice with a little saturated aqueous sodium bicarbonate solution. The aqueous phase was re-extracted twice with dichloromethane. The combined organic phases were dried over sodium sulfate, filtered, concentrated and lyophilized. This gave 62 mg of the target compound (79% of theory).

LC-MS (Method 5): Rₜ=0.66 min

MS (ESpos): m/z=436 (M+H)⁺

¹H-NMR (400 MHz, DMSO-d₆): δ [ppm]=1.10 (s, 3H), 1.69 (br. s, 2H), 1.87-2.03 (m, 2H), 5.32 (s, 2H), 6.09-6.43 (m, 1H), 7.18-7.26 (m, 2H), 7.42 (d, 1H), 7.54-7.63 (m, 3H), 7.68 (d, 1H), 8,67 (t, 1H), 8.88 (d, 1H), [further signal under solvent peak].

### Example 8

### 8-[(2,6-Difluorobenzyl)oxy]-N-[2-(1-hydroxycyclopentyl)ethyl]quinoline-4-carboxamide

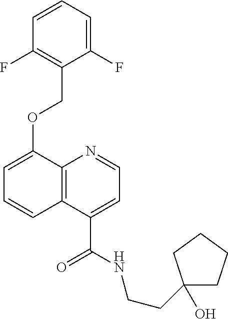 [SMILES: O=C(NCCC1(O)CCCC1)c1ccnc2c(OCc3c(F)cccc3F)cccc12]

12.9 mg (0.1 mmol) of 1-(2-aminoethyl)cyclopentanol were initially charged in a 96-well deep well multititre plate. A solution of 31.5 mg (0.1 mmol) of 8-[(2,6-difluorobenzyl)oxy]quinoline-4-carboxylic acid from Example 4A in 0.4 ml of DMF and a solution of 45.6 mg (0.12 mol) of HATU in 0.4 ml of DMF were successively added thereto. After adding 20.2 mg (0.2 mmol) of 4-methylmorpholine, the mixture was shaken at RT overnight. Then the mixture was filtered and the target compound was isolated from the filtrate by preparative LC-MS (Method 12). The product-containing fractions were concentrated under reduced pressure using a centrifugal dryer. The residue of each product fraction was dissolved in 0.6 ml of DMSO. These were combined and finally freed of the solvent in a centrifugal dryer. This gave 30 mg (68% of theory).

LC-MS (Method 13): Rₜ=1.00 min

MS (ESpos): m/z=427 (M+H)⁺

In analogy to Example 8, the example compounds shown in Table 1 were prepared by reacting 8-[(2,6-difluorobenzyl)oxy]quinoline-4-carboxylic acid from Example 4A with the appropriate amines, which are commercially available or described above, under the conditions described:

TABLE 1

| | IUPAC name/structure | |
|---|---|---|
| Example | (Yield) | Analytical data |
| 9 | 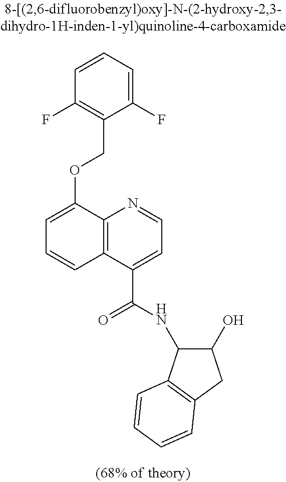 [SMILES: O=C(NC1c2ccccc2CC1O)c1ccnc2c(OCc3c(F)cccc3F)cccc12] | LC-MS (Method 13): Rₜ = 1.04 min MS (ESpos): m/z = 447 (M + H)⁺ |
| 10 | 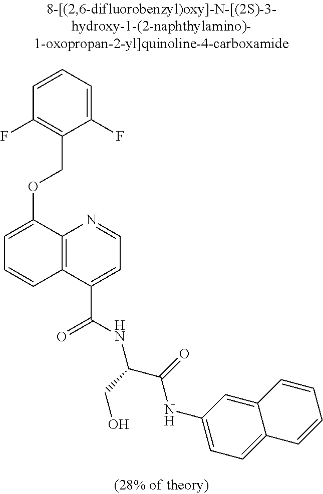 [SMILES: O=C(N[C@@H](CO)C(=O)Nc1ccc2ccccc2c1)c1ccnc2c(OCc3c(F)cccc3F)cccc12] | LC-MS (Method 13): Rₜ = 1.06 min MS (ESpos): m/z = 528 (M + H)⁺ |
| 11 | 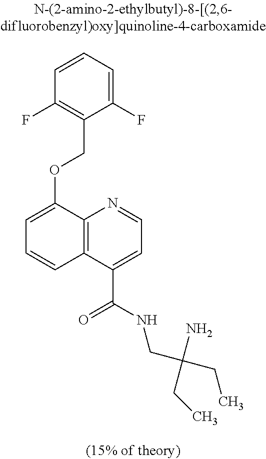 [SMILES: CCC(N)(CC)CNC(=O)c1ccnc2c(OCc3c(F)cccc3F)cccc12] | LC-MS (Method 13): Rₜ = 0.74 min MS (ESpos): m/z = 414 (M + H)⁺ |
| 12 | 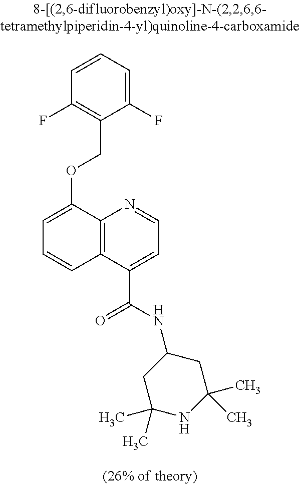 [SMILES: CC1(C)CC(NC(=O)c2ccnc3c(OCc4c(F)cccc4F)cccc23)CC(C)(C)N1] | LC-MS (Method 13): Rₜ = 0.74 min MS (ESpos): m/z = 454 (M + H)⁺ |
| 13 | 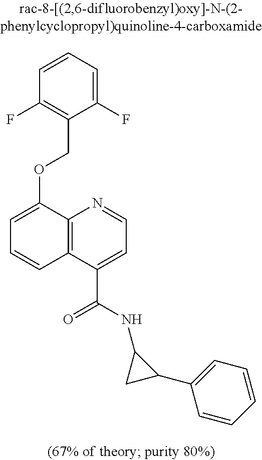 [SMILES: O=C(NC1CC1c1ccccc1)c1ccnc2c(OCc3c(F)cccc3F)cccc12] | LC-MS (Method 13): Rₜ = 1.14 min MS (ESpos): m/z = 431 (M + H)⁺ |
| 14 | 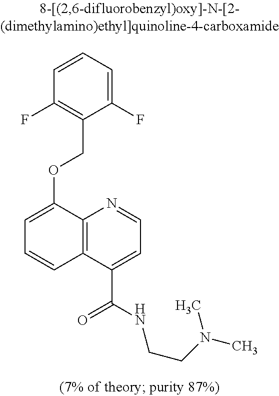 [SMILES: CN(C)CCNC(=O)c1ccnc2c(OCc3c(F)cccc3F)cccc12] | LC-MS (Method 13): Rₜ = 0.69 min MS (ESpos): m/z = 386 (M + H)⁺ |
| 15 | 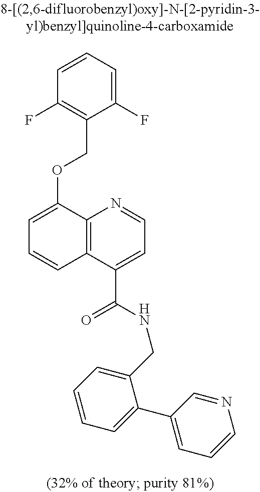 [SMILES: O=C(NCc1ccccc1-c1cccnc1)c1ccnc2c(OCc3c(F)cccc3F)cccc12] | LC-MS (Method 13): Rₜ = 1.03 min MS (ESpos): m/z = 482 (M + H)⁺ |
| 16 | 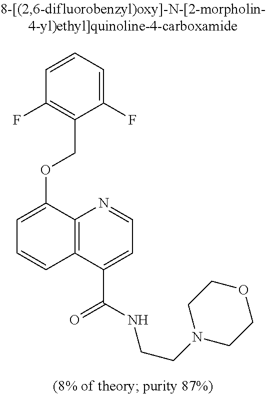 [SMILES: O=C(NCCN1CCOCC1)c1ccnc2c(OCc3c(F)cccc3F)cccc12] | LC-MS (Method 13): Rₜ = 0.70 min MS (ESpos): m/z = 428 (M + H)⁺ |
| 17 | 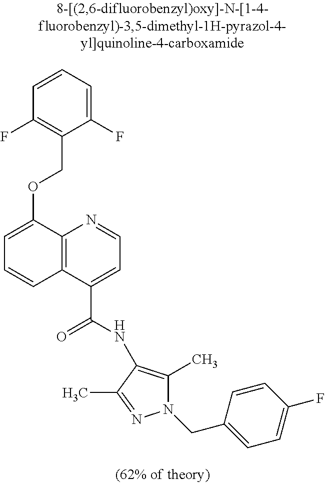 [SMILES: Cc1nn(Cc2ccc(F)cc2)c(C)c1NC(=O)c1ccnc2c(OCc3c(F)cccc3F)cccc12] | LC-MS (Method 13): Rₜ = 1.11 min MS (ESpos): m/z = 517 (M + H)⁺ |
| 18 | 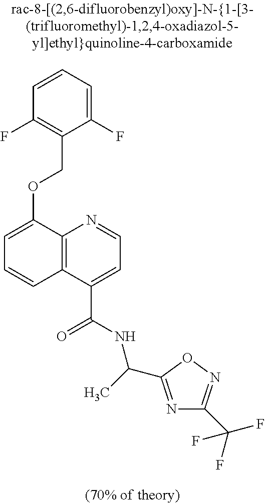 [SMILES: CC(NC(=O)c1ccnc2c(OCc3c(F)cccc3F)cccc12)c1nc(C(F)(F)F)no1] | LC-MS (Method 13): Rₜ = 1.15 min MS (ESpos): m/z = 479 (M + H)⁺ |
| 19 | 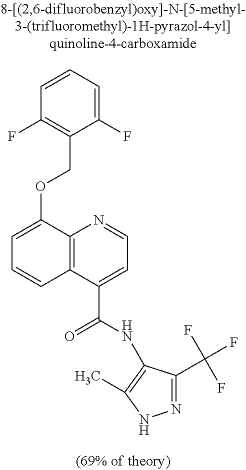 [SMILES: Cc1[nH]nc(C(F)(F)F)c1NC(=O)c1ccnc2c(OCc3c(F)cccc3F)cccc12] | LC-MS (Method 13): Rₜ = 1.02 min MS (ESpos): m/z = 463 (M + H)⁺ |
| 20 | 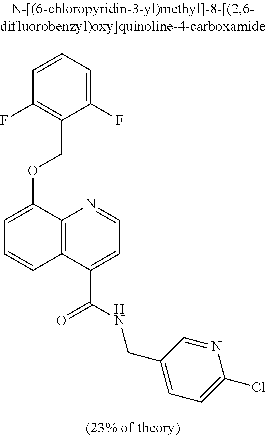 [SMILES: O=C(NCc1ccc(Cl)nc1)c1ccnc2c(OCc3c(F)cccc3F)cccc12] | LC-MS (Method 13): Rₜ = 1.05 min MS (ESpos): m/z = 440 (M + H)⁺ |
| 21 | 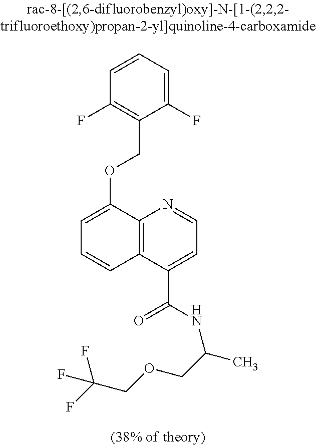 [SMILES: CC(COCC(F)(F)F)NC(=O)c1ccnc2c(OCc3c(F)cccc3F)cccc12] | LC-MS (Method 13): Rₜ = 1.09 min MS (ESpos): m/z = 455 (M + H)⁺ |
| 22 | 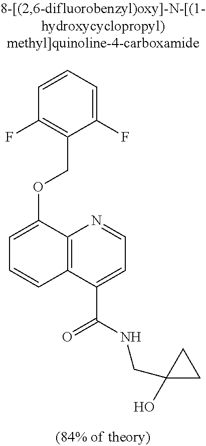 [SMILES: O=C(NCC1(O)CC1)c1ccnc2c(OCc3c(F)cccc3F)cccc12] | LC-MS (Method 13): Rₜ = 0.90 min MS (ESpos): m/z = 385 (M + H)⁺ |
| 23 | 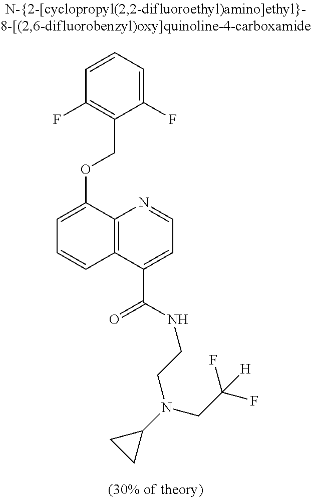 [SMILES: O=C(NCCN(CC(F)F)C1CC1)c1ccnc2c(OCc3c(F)cccc3F)cccc12] | LC-MS (Method 13): Rₜ = 1.11 min MS (ESpos): m/z = 462 (M + H)⁺ |
| 24 | 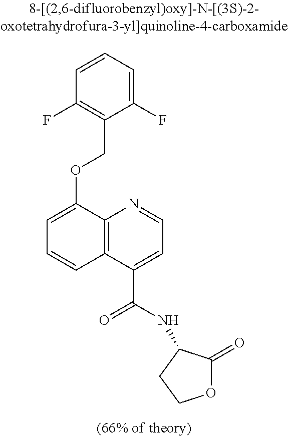 [SMILES: O=C(N[C@H]1CCOC1=O)c1ccnc2c(OCc3c(F)cccc3F)cccc12] | LC-MS (Method 13): Rₜ = 0.93 min MS (ESpos): m/z = 399 (M + H)⁺ |
| 25 | 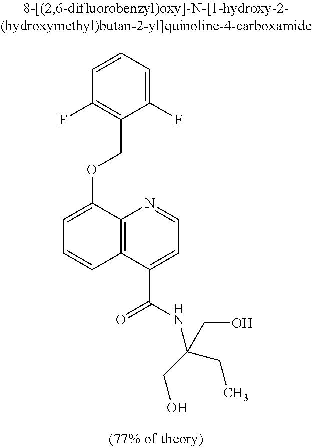 [SMILES: CCC(CO)(CO)NC(=O)c1ccnc2c(OCc3c(F)cccc3F)cccc12] | LC-MS (Method 13): Rₜ = 0.89 min MS (ESpos): m/z = 417 (M + H)⁺ |
| 26 | 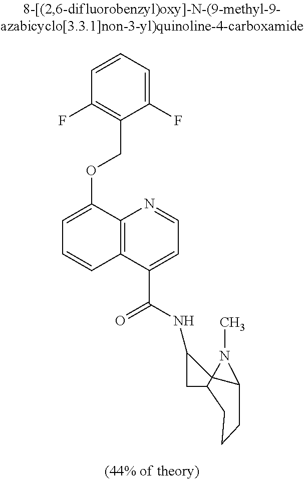 [SMILES: CN1C2CCCC3CC(NC(=O)c4ccnc5c(OCc6c(F)cccc6F)cccc45)C321] | LC-MS (Method 13): Rₜ = 0.72 min MS (ESpos): m/z = 452 (M + H)⁺ |
| 27 | 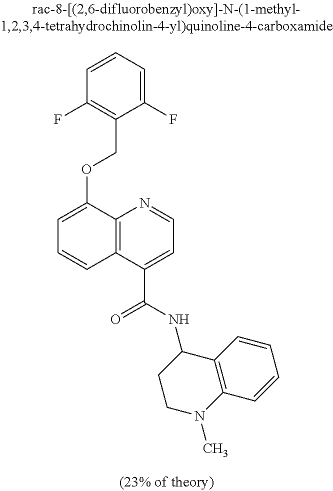 [SMILES: CN1CCC(NC(=O)c2ccnc3c(OCc4c(F)cccc4F)cccc23)c2ccccc21] | LC-MS (Method 13): Rₜ = 1.15 min MS (ESpos): m/z = 460 (M + H)⁺ |
| 28 | 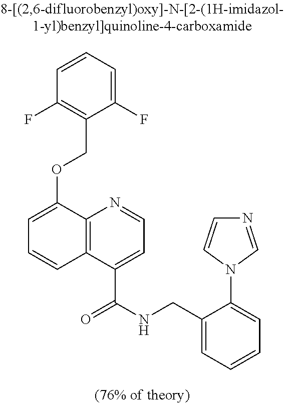 [SMILES: O=C(NCc1ccccc1-n1ccnc1)c1ccnc2c(OCc3c(F)cccc3F)cccc12] | LC-MS (Method 13): Rₜ = 0.79 min MS (ESpos): m/z = 471 (M + H)⁺ |
| 29 | 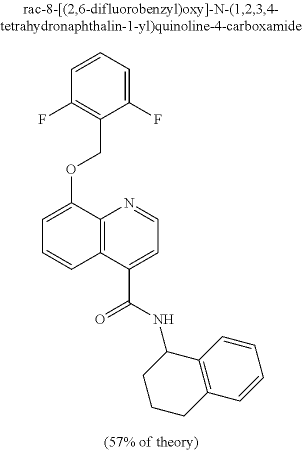 [SMILES: O=C(NC1CCCc2ccccc21)c1ccnc2c(OCc3c(F)cccc3F)cccc12] | LC-MS (Method 13): Rₜ = 1.17 min MS (ESpos): m/z = 445 (M + H)⁺ |
| 30 | 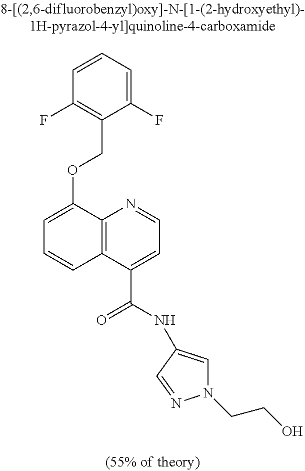 [SMILES: O=C(Nc1cnn(CCO)c1)c1ccnc2c(OCc3c(F)cccc3F)cccc12] | LC-MS (Method 13): Rₜ = 0.89 min MS (ESpos): m/z = 425 (M + H)⁺ |
| 31 | 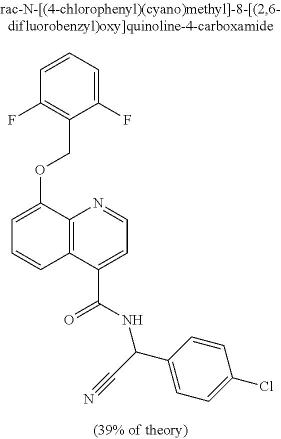 [SMILES: N#CC(NC(=O)c1ccnc2c(OCc3c(F)cccc3F)cccc12)c1ccc(Cl)cc1] | LC-MS (Method 13): Rₜ = 1.17 min MS (ESpos): m/z = 464 (M + H)⁺ |

### Example 32

### ent-8-[(2,6-Difluorobenzyl)oxy]-N-(6,6,7,7,7-pentafluoro-2-hydroxy-2-methylheptan-3-yl)quinoline-4-carboxamide (Enantiomer A)

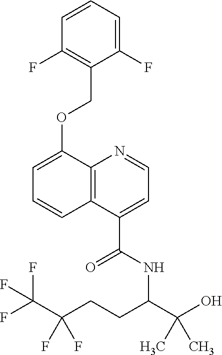 [SMILES: CC(C)(O)C(CCC(F)(F)C(F)(F)F)NC(=O)c1ccnc2c(OCc3c(F)cccc3F)cccc12]

50 mg (0.16 mmol) of 8-[(2,6-difluorobenzyl)oxy]quinoline-4-carboxylic acid from Example 4A were dissolved in 0.50 ml of DMF, 78 mg (0.21 mmol) of HATU and 0.14 ml (0.79 mmol) of N,N-diisopropylethylamine were added and the mixture was stirred at room temperature for 20 min. Subsequently, 56 mg (0.21 mmol, purity 94%) of ent-3-amino-6,6,7,7,7-pentafluoro-2-methylheptan-2-ol hydrochloride (Enantiomer A) from Example 30A were added. The mixture was stirred at RT for 1 h, water, acetonitrile and a little TFA were subsequently added and the reaction mixture was purified by preparative HPLC (RP18 column, mobile phase: acetonitrile/water gradient). The product fractions were combined, concentrated by evaporation and lyophilized using acetonitrile/water. This gave 75 mg of the title compound (83% of theory; purity 93%).

LC-MS (Method 11):=1.34 min

MS (ESpos): m/z=533 (M+H)⁺

¹H-NMR (400 MHz, DMSO-d₆): δ [ppm]=1.14 (s, 3H), 1.24 (s, 3H), 1.60-1.72 (m, 1H), 2.02-2.13 (m, 1H), 2.15-2.35 (m, 2H), 4.00-4.08 (m, 1H), 4.63 (s, 1H), 5.34 (s, 2H), 7.19-7.26 (m, 211), 7.44 (dd, 111), 7.53 (d, 1H), 7.54-7.65 (m, 311), 8.47 (d, 1H), 8.89 (d, 1H).

Analogously to Example 32, the example compounds shown in Table 2 were prepared by reacting 8-[(2,6-difluorobenzyl)oxy]quinoline-4-carboxylic acid from Example 4A with the appropriate commercially available or above-described amines (1.1-5 equivalents), HATU (1.1-4.5 equivalents) and N,N-diisopropylethylamine (3-12 equivalents) in DMF or in DMF/dichloromethane (1/1) under the reaction conditions described (reaction time: 1-48 h; temperature: 0° C.-RT, −20° C., RT or 60° C.).

Exemplary Work-Up of the Reaction Mixture:

The reaction mixture was diluted with water and purified by preparative HPLC (RP18 column, mobile phase: acetonitrile/water gradient).

TABLE 2

| | IUPAC name/structure | |
|---|---|---|
| Example | (Yield) | Analytical data |
| 33 | 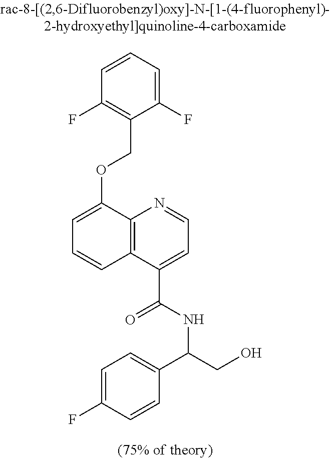 [SMILES: O=C(NC(CO)c1ccc(F)cc1)c1ccnc2c(OCc3c(F)cccc3F)cccc12] | ¹H-NMR (400 MHz, DMSO-d₆): δ [ppm] = 3.65 (d, 2H), 5.02 (br. s, 1H), 5.17 (q, 1H), 5.33 (s, 2H), 7.16-7.26 (m, 3H), 7.41-7.49 (m, 3H), 7.53- 7.62 (m, 3H), 7.65 (dd, 1H), 8.88 (d, 1H), 9.13 (d, 1H). LC-MS (Method 11): Rₜ = 1.15 min MS (ESpos): m/z = 453 (M + H)⁺ |
| 34 | 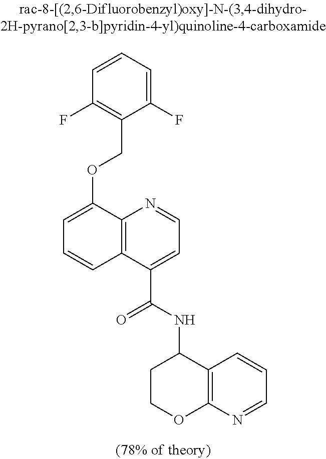 [SMILES: O=C(NC1CCOc2ncccc21)c1ccnc2c(OCc3c(F)cccc3F)cccc12] | ¹H-NMR (400 MHz, DMSO-d₆): δ [ppm] = 2.04-2.14 (m, 1H), 2.19- 2.29 (m, 1H), 4.35-4.45 (m, 2H), 5.34 (s, 2H), 5.39 (q, 1H), 7.03 (dd, 1H), 7.19-7.26 (m, 2H), 7.43-7.48 (m, 1H), 7.53-7.65 (m, 3H), 7.75- 7.83 (m, 2H), 8.10 (dd, 1H), 8.88 (d, 1H), 9.24 (d, 1H). LC-MS (Method 11): Rₜ = 1.07 min MS (ESpos): m/z = 448 (M + H)⁺ |
| 35 | 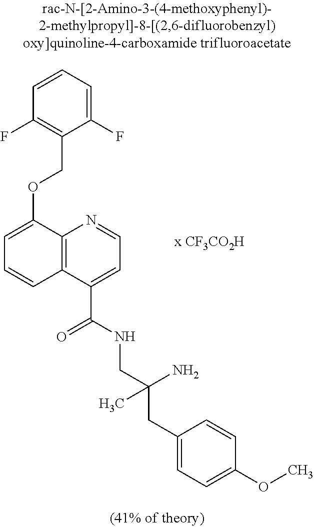 [SMILES: COc1ccc(CC(C)(N)CNC(=O)c2ccnc3c(OCc4c(F)cccc4F)cccc23)cc1] | LC-MS (Method 11): Rₜ = 0.97 min MS (ESpos): m/z = 492 (M − TFA + H)⁺ |

### Example 36

### ent-N-[2-Amino-3-(4-methoxyphenyl)-2-methylpropyl]-8-[(2,6-difluorobenzyl)oxy]quinoline-4-carboxamide (Enantiomer A)

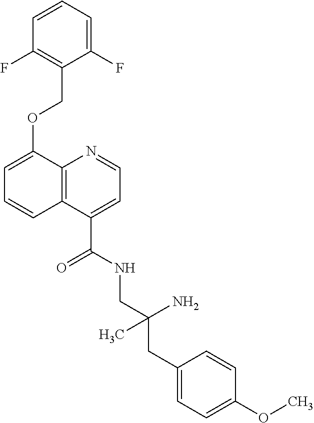 [SMILES: COc1ccc(CC(C)(N)CNC(=O)c2ccnc3c(OCc4c(F)cccc4F)cccc23)cc1]

78 mg of rac-N-[2-amino-3-(4-methoxyphenyl)-2-methylpropyl1-8-[(2,6-difluorobenzyl)oxy]quinoline-4-carboxamide trifluoroacetate from Example 35 were separated into the enantiomers by preparative separation on the chiral phase [column: Daicel Chiralpak AY-H, 5 μm, 250×20 mm; mobile phase: 50% isohexane, 50% ethanol+0.2% diethylamine; flow rate: 15 ml/min, temperature: 40° C.; detection: 220 nm].

Enantiomer A: yield: 21 mg (99% ee)

Rₜ=6.65 min [Chiralpak AY-H, 5 μm, 250×4.6 mm; mobile phase: 50% isohexane, 50% ethanol+0.2% diethylamine; flow rate: 1 ml/min; temperature: 40° C.; detection: 235 nm].

¹H-NMR (400 MHz, DMSO-d₆): δ [ppm]=0.97 (s, 3H), 1.38 (br. s, 2H), 2.61 (s, 2H), 3.17-3.34 (m, 2H, superposed by solvent peak), 3.73 (s, 3H), 5.32 (s, 2H), 6.87 (d, 2H), 7.14-7.28 (m, 4H), 7.42 (d, 1H), 7.53-7.63 (m, 3H), 7.71 (d, 1H), 8.58 (t, 1H), 8.88 (d, 1H).

### Example 37

### ent-N-[2-Amino-3-(4-methoxyphenyl)-2-methylpropyl]-8-[(2,6-difluorobenzyl)oxy]quinoline-4-carboxamide (Enantiomer B)

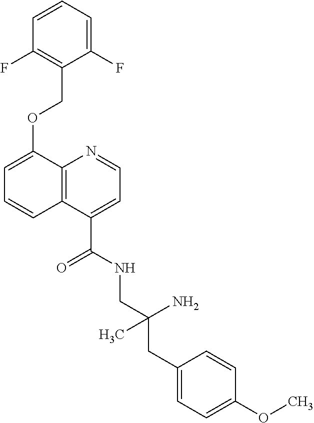 [SMILES: COc1ccc(CC(C)(N)CNC(=O)c2ccnc3c(OCc4c(F)cccc4F)cccc23)cc1]

78 mg of rac-N-[2-amino-3-(4-methoxyphenyl)-2-methylpropyl]-8-[(2,6-difluorobenzyl)oxy]quinoline-4-carboxamide trifluoroacetate from Example 35 were separated into the enantiomers by preparative separation on the chiral phase [column: Daicel Chiralpak AY-H, 5 μm, 250×20 mm; mobile phase: 50% isohexane, 50% ethanol+0.2% diethylamine; flow rate: 15 ml/min, temperature: 40° C.; detection: 220 nm].

Enantiomer B: yield: 30 mg (99% ee)

Rₜ=9.82 min [Chiralpak AY-H, 5 μm, 250×4.6 mm; mobile phase: 50% isohexane, 50% ethanol+0.2% diethylamine; flow rate: 1 ml/min; temperature: 40° C.; detection: 235 nm].

¹H-NMR (400 MHz, DMSO-d₆): δ [ppm]=0.97 (s, 3H), 1.38 (br. s, 2H), 2.61 (s, 2H), 3.17-3.34 (m, 2H, superposed by solvent peak), 3.73 (s, 3H), 5.32 (s, 2H), 6.86 (d, 2H), 7.14-7.27 (m, 4H), 7.42 (d, 1H), 7.53-7.63 (m, 3H), 7.71 (d, 1H), 8.58 (t, 1H), 8.88 (d, 1H).

### Example 38

### 8-[(2,6-Difluorobenzyl)oxy]-N-[(2R)-1-hydroxyhexan-2-yl]quinoline-3-carboxamide

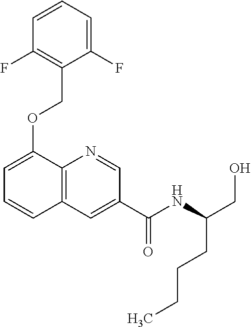 [SMILES: CCCC[C@H](CO)NC(=O)c1cnc2c(OCc3c(F)cccc3F)cccc2c1]

70 mg (0.21 mmol; purity 94%) of 8-[(2,6-difluorobenzyl)oxy]quinoline-3-carboxylic acid from Example 32A were dissolved in 1 ml of DMF, 87 mg (0.23 mmol) of HATU and 0.15 ml (0.84 mmol) of N,N-diisopropylethylamine were added and the mixture was stirred at room temperature for 20 min. 27 mg (0.23 mmol) of (2R)-2-aminohexan-1-ol were then added. The mixture was stirred at RT for 40 min, water, acetonitrile and TFA were subsequently added and the reaction mixture was purified by preparative HPLC (RP18 column, mobile phase: acetonitrile/water gradient with addition of 0.1% TFA). The product fractions were combined and concentrated. Subsequently, the residue was taken up in dichloromethane and a little methanol, and washed twice with a little saturated aqueous sodium bicarbonate solution. The aqueous phase was re-extracted twice with dichloromethane. The combined organic phases were dried over sodium sulfate, filtered and concentrated. 57 mg of the title compound were obtained (65% of theory).

LC-MS (Method 5): Rₜ=0.99 min

MS (ESpos): m/z=415 (M+H)⁺

¹H-NMR (400 MHz, DMSO-d₆): δ [ppm]=0.89 (t, 3H), 1.24-1.41 (m, 4H), 1.43-1.58 (m, 1H), 1.60-1.72 (m, 1H), 3.38-3.53 (m, 2H), 3.95-4.07 (m, 1H), 4.73 (t, 1H), 5.34 (s, 2H), 7.19-7.27 (m, 2H), 7.49 (d, 1H), 7.54-7.70 (m, 3H), 8.38 (d, 1H), 8.78 (d, 1H), 9.18 (d, 1H).

### Example 39

### 8-[(2,6-Difluorobenzyl)oxy]-N-[1-(4-fluorophenyl)cyclopropyl]quinoline-3-carboxamide

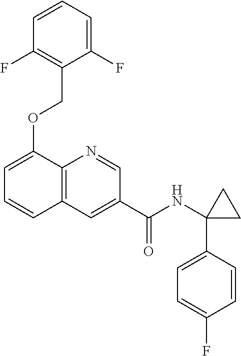 [SMILES: O=C(NC1(c2ccc(F)cc2)CC1)c1cnc2c(OCc3c(F)cccc3F)cccc2c1]

The target compound was obtained by reacting 8-[(2,6-difluorobenzyl)oxy]quinoline-3-carboxylic acid from Example 32A analogously to Example 38 with 1-(4-fluorophenyl)cyclopropanamine [yield: 15% of theory].

LC-MS (Method 5): Rₜ=1.09 min

MS (ESpos): m/z=449 (M+H)⁺

¹H-NMR (500 MHz, DMSO-d₆): δ [ppm]=1.25-1.35 (m, 4H), 5.35 (s, 2H), 7.07-7.14 (m, 2H), 7.19-7.27 (m, 2H), 7.29-7.34 (m, 2H), 7.50 (d, 1H), 7.54-7.70 (m, 3H), 8.79-8.84 (m, 1H), 9.15-9.20 (m, 1H), 9.50-9.56 (m, 1H).

### Example 40

### ent-N-(2-Amino-5,5,5-trifluoro-2-methylpentyl)-8-[(2,6-difluorobenzyl)oxy]quinoline-3-carboxamide (Enantiomer B)

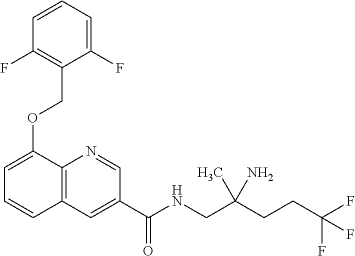 [SMILES: CC(N)(CCC(F)(F)F)CNC(=O)c1cnc2c(OCc3c(F)cccc3F)cccc2c1]

The target compound was obtained by reacting ent-benzyl {1-[({8-[(2,6-difluorobenzyl)oxy]quinolin-3-yl carbonyl)amino]-5,5,5-trifluoro-2-methylpentan-2-yl}carbamate trifluoroacetate (Enantiomer B) from Example 33A analogously to Example 6 [yield: 81% of theory].

LC-MS (Method 5): Rₜ=0.73 min

MS (ESpos): m/z=468 (M+H)⁺

¹H-NMR (400 MHz, DMSO-d₆): δ [ppm]=1.03 (s, 3H), 1.47-1.58 (m, 2H), 1.62 (br. s, 2H), 2.23-2.48 (m, 2H), 3.24-3.31 (in, 2H, partially superposed by solvent peak), 5.33 (s, 2H), 7.18-7.27 (m, 2H), 7.48 (d, 1H), 7.53-7.68 (m, 2H), 7.70 (d, 1H), 8.67 (t, 1H), 8.78 (d, 1H), 9.17 (d, 1H).

### Example 41

### N-[(2S)-2-Amino-2-methylbutyl]-8-[(2,6-difluorobenzyl)oxy]quinoline-3-carboxamide

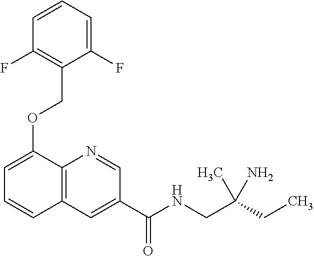 [SMILES: CC[C@](C)(N)CNC(=O)c1cnc2c(OCc3c(F)cccc3F)cccc2c1]

The target compound was obtained by reacting benzyl {(2S)-1-[({8-[(2,6-difluorobenzyl)oxy]quinolin-3-yl}carbonyl)amino]-2-methylbutan-2-yl}carbamate trifluoroacetate from Example 34A analogously to Example 6 [yield: 83% of theory].

LC-MS (Method 5): Rₜ=0.69 min

MS (ESpos): m/z=400 (M+H)⁺

¹H-NMR (400 MHz, DMSO-d₆): δ [ppm]=0.88 (t, 3H), 0.98 (s, 3H), 1.30-1.43 (m, 2H), 1.62 (br. s, 2H), 3.22-3.31 (m, 2H, partially superposed by solvent peak), 5.33 (s, 2H), 7.18-7.27 (in, 2H), 7.48 (d, 1H), 7.53-7.68 (m, 2H), 7.70 (d, 1H), 8.45-8.58 (m, 1H), 8.78 (d, 1H), 9.17 (d, 1H).

### Example 42

### rac-N-(2-Amino-2-cyclopropylpropyl)-8-[(2,6-difluorobenzyl)oxy]quinoline-4-carboxamide

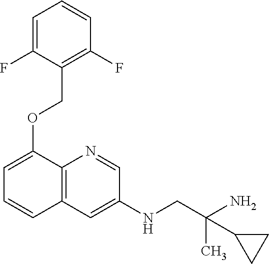 [SMILES: CC(N)(CNc1cnc2c(OCc3c(F)cccc3F)cccc2c1)C1CC1]

50 mg (0.16 mmol) of 8-[(2,6-difluorobenzyl)oxy]quinoline-4-carboxylic acid from Example 4A were dissolved in 0.53 ml of DMF, 78 mg (0.21 mmol) of HATU and 0.17 ml (0.95 mmol) of N,N-diisopropylethylamine were added and the mixture was stirred at room temperature for 20 min. 39 mg (0.21 mmol) of rac-2-cyclopropylpropane-1,2-diamine dihydrochloride were then added, and the mixture was stirred at RT for 30 minutes. Another 78 mg (0.21 mmol) of HATU, 0.06 ml (0.32 mmol) of N,N-diisopropylethylamine and 39 mg (0.21 mmol) of rac-2-cyclopropylpropane-1,2-diamine dihydrochloride were added, and the mixture was stirred at RT for 30 minutes. Acetonitrile, water and TFA were added and the reaction solution was purified by preparative HPLC (RP18 column, mobile phase: acetonitrile/water gradient with addition of 0.1% TFA). The product fractions were combined and concentrated. The residue was taken up in dichloromethane and washed twice with saturated aqueous sodium bicarbonate solution. The combined aqueous phases were reextracted twice with dichloromethane. The combined organic phases were dried over sodium sulfate, filtered and concentrated. This gave 31 mg of the title compound (47% of theory; purity 99%).

LC-MS (Method 5): Rₜ=0.59 min

MS (ESpos): m/z=412 (M+H)⁺

¹H-NMR (400 MHz, DMSO-d₆): δ [ppm]=0.18-0.38 (m, 4H), 0.81-0.90 (m, 1H), 1.00 (s, 3H), 1.15-1.35 (s br., 2H), 3.34-3.40 (in, 2H, partially superposed by solvent peak), 5.33 (s, 2H), 7.19-7.27 (m, 2H), 7.43 (d, 1H), 7.53-7.63 (m, 3H), 7.72 (d, 1H), 8.55 (t, 1H), 8.78 (d, 1H).

### Example 43

### ent-N-(2-Amino-5,5,5-trifluoro-2-methylpentyl)-8-[(2,6-difluorobenzyl)oxy]-2-methylquinoline-4-carboxamide (Enantiomer B)

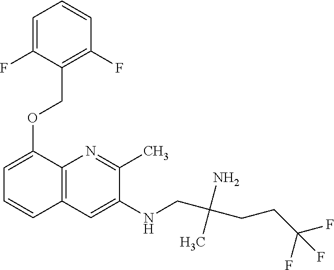 [SMILES: Cc1nc2c(OCc3c(F)cccc3F)cccc2cc1NCC(C)(N)CCC(F)(F)F]

60 mg (0.08 mmol) of ent-benzyl {1-[({8-[(2,6-difluorobenzyl)oxy]-2-methylquinolin-4-yl}carbonyl)amino]-5,5,5-trifluoro-2-methylpentan-2-yl}carbamate trifluoroacetate (Enantiomer B) from Example 44A were dissolved in 8.7 ml of ethanol, 19 μl of TFA and 2.6 mg of palladium on activated carbon (10%) were added and the mixture was hydrogenated at atmospheric pressure for 4.5 hours. The reaction mixture was filtered through a Millipore filter and washed through with ethanol, and the filtrate was concentrated. The residue was dissolved in 8.7 ml of ethanol, 19 μl of TFA and 2.6 mg of palladium on activated carbon (10%) were added and the mixture was hydrogenated at atmospheric pressure for one hour. The reaction solution was filtered through a Millipore filter and washed through with ethanol, and the filtrate was concentrated under reduced pressure. Acetonitrile, water and TFA were added to the residue and the product was purified by preparative HPLC (RP18 column, mobile phase: acetonitrile/water gradient with addition of 0.1% TFA). The product fractions were combined and concentrated. Subsequently, the residue was taken up in dichloromethane and washed twice with saturated aqueous sodium bicarbonate solution. The combined aqueous phases were reextracted twice with dichloromethane. The combined organic phases were dried over sodium sulfate, filtered and concentrated. This gave 25 mg of the target compound (63% of theory).

LC-MS (Method 5): Rₜ=0.70 min

MS (ESpos): m/z=482 (M+H)⁺

¹-NMR (400 MHz, DMSO-d₆): δ [ppm]=1.03 (s, 3H), 1.24 (s, 1H), 1.47-1.64 (m, 4H), 2.26-2.48 (m, 2H), 2.64 (s, 3H), 3.21-3.32 (m, 2H, partially superposed by solvent peak), 5.33 (s, 2H), 7.17-7.26 (m, 2H), 7.37 (d, 1H), 7.43-7.49 (m, 2H), 7.53-7.64 (m, 2H), 8.63 (t, 1H).

### Example 44

### cis/trans-N-[(4-Cyanocyclohexyl)methyl]-8-[2,6-difluorobenzyl)oxy]quinoline-4-carboxamide

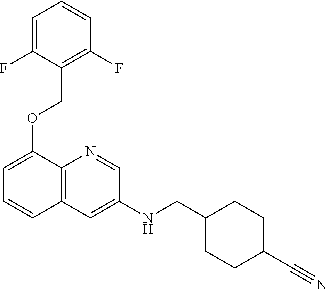 [SMILES: N#CC1CCC(CNc2cnc3c(OCc4c(F)cccc4F)cccc3c2)CC1]

50 mg (0.16 mmol) of 8-[(2,6-difluorobenzyl)oxy]quinoline-4-carboxylic acid from Example 4A were dissolved in 0.53 ml of DMF, 78 mg (0.21 mmol) of HATU and 0.17 ml (0.95 mmol) of N,N-diisopropylethylamine were added and the mixture was stirred at room temperature for 20 min. 28.5 mg (0.21 mmol) of cisfirans-4-(aminomethyl)cyclohexanecarbonitrile were then added, and the mixture was stirred at RT for 30 minutes. Acetonitrile, water and TFA were added and the reaction solution was purified by preparative HPLC (RP18 column, mobile phase: acetonitrile/water gradient with addition of 0.1% TFA). The product fractions were combined and concentrated. The residue was taken up in dichloromethane and washed twice with saturated aqueous sodium bicarbonate solution. The combined aqueous phases were reextracted twice with dichloromethane. The combined organic phases were dried over sodium sulfate, filtered and concentrated. This gave 43 mg of the title compound (61% of theory; purity 98%).

LC-MS (Method 5): Rₜ=0.93 min

MS (ESpos): m/z=436 (M+H)⁺

¹H-NMR (400 MHz, DMSO-d₆): δ [ppm]=0.97-1.11 (m, 1H), 1.14-1.33 (m, 1H), 1.43-1.72 (m, 3H), 1.73-1.94 (m, 3H), 2.00-2.09 (m, 1H), 2.61-2.70 (m, 1H), 3.15-3.27 (m, 2H), 5.33 (s, 2H), 7.18-7.27 (m, 2H), 7.43 (d, 1H), 7.49-7.62 (m, 3H), 7.68 (t, 1H), 8.69-8.80 (m, 1H), 8.76 (d, 1H).

### Example 45

### ent-N-(2-Amino-5,5,5-trifluoro-2-methylpentyl)-8-[(2,6-difluorobenzyl)oxy]-6-methylquinoline-4-carboxamide (Enantiomer B)

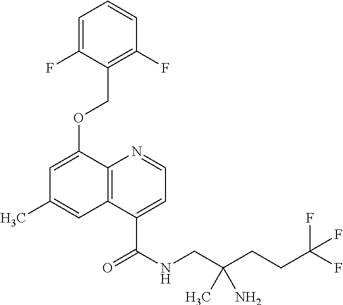 [SMILES: Cc1cc(OCc2c(F)cccc2F)c2nccc(C(=O)NCC(C)(N)CCC(F)(F)F)c2c1]

160 mg (0.243 mmol) of ent-benzyl {1-[({8-[(2,6-difluorobenzyl)oxy]-6-methylquinolin-4-yl}carbonyl)amino]-5,5,5-trifluoro-2-methylpentan-2-yl}carbamate (Enantiomer B) from Example 39A were dissolved in 6.2 ml of ethanol, 94 μl of TFA and 7.8 mg of palladium on activated carbon (10%) were added and the mixture was hydrogenated at atmospheric pressure for 4 hours. The reaction mixture was filtered and the filtrate was concentrated. The residue was dissolved in 6.2 ml of ethanol, 94 μl of TFA and 7.8 mg of palladium on activated carbon (10%) were added and the mixture was hydrogenated at atmospheric pressure for one hour. The reaction solution was filtered through a Millipore filter and the filtrate was concentrated. The residue was purified by preparative HPLC (RP18 column, mobile phase: acetonitrile/water gradient with addition of 0.1% TFA). The product fractions were combined and concentrated. Subsequently, the residue was taken up in dichloromethane and washed twice with saturated aqueous sodium bicarbonate solution. The combined aqueous phases were reextracted twice with dichloromethane. The combined organic phases were dried over sodium sulfate, filtered and concentrated. This gave 79 mg of the target compound (67% of theory).

LC-MS (Method 5): Rₜ=0.75 min

MS (ESpos): m/z=482 (M+H)⁺

¹H-NMR (400 MHz, DMSO-d₆): δ [ppm]=1.04 (s, 3H), 1.49-1.79 (m, 4H), 2.26-2.47 (m, 2H), 2.48 (s, 3H), 3.25-3.30 (d, 2H, partially superposed by solvent peak), 5.31 (s, 2H), 7.19-7.27 (m, 2H), 7.31 (s, 1H), 7.44 (s, 1H), 7.48 (d, 1H), 7.52-7.63 (m, 1H), 8.63 (t, 1H), 8.78 (d, 1H).

### Example 46

### ent-8-[(2,6-Difluorobenzyl)oxy]-N-[(2R)-1-hydroxyhexan-2-yl]-2-methylquinoline-4-carboxamide (Enantiomer)

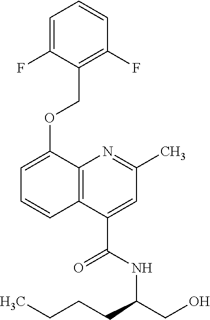 [SMILES: CCCC[C@H](CO)NC(=O)c1cc(C)nc2c(OCc3c(F)cccc3F)cccc12]

30 mg (0.16 mmol) of 8-[(2,6-difluorobenzyl)oxy]-2-methylquinoline-4-carboxylic acid from Example 43A were dissolved in 0.31 ml of DMF, 45 mg (0.12 mmol) of HATU and 79 μl (0.46 mmol) of N,N-diisopropylethylamine were added and the mixture was stirred at room temperature for 20 min. 14 mg (0.12 mmol) of (2R)-2-aminohexan-1-ol were then added, and the mixture was stirred at RT for 2 hours. 17 mg (0.046 mmol) of HATU, 32 μl (0.182 mmol) of N,N-diisopropylethylamine and, after 10 minutes, 5.3 mg (0.046 mmol) of (2R)-2-aminohexan-1-ol were added, and the mixture was stirred at RT for 3 days. Acetonitrile, water and TFA were added and the reaction solution was purified by preparative HPLC (RP18 column, mobile phase: acetonitrile/water gradient with addition of 0.1% TFA). The product-containing fractions were combined and concentrated. The residue was taken up in dichloromethane and washed twice with saturated aqueous sodium bicarbonate solution. The combined aqueous phases were reextracted twice with dichloromethane. The combined organic phases were dried over sodium sulfate, filtered and concentrated. More acetonitrile, water and TFA were added and the substance was purified by preparative HPLC (RP18 column, mobile phase: acetonitrile/water gradient with addition of 0.1% TFA). The product-containing fractions were combined and concentrated. The residue was taken up in dichloromethane and washed twice with saturated aqueous sodium bicarbonate solution. The combined aqueous phases were reextracted twice with dichloromethane. The combined organic phases were dried over sodium sulfate, filtered, concentrated and lyophilized. This gave 17 mg of the title compound (42% of theory; purity 97%).

LC-MS (Method 5): Rₜ=0.86 min

MS (ESpos): m/z=429 (M+H)⁺

¹H-NMR (400 MHz, DMSO-d₆): δ [ppm]=0.85-0.94 (m, 3H), 1.26-1.45 (m, 5H), 1.58-1.68 (m, 1H), 2.64 (s, 3H), 3.38-3.53 (m, 2H), 3.95-4.06 (m, 1H), 4.74 (br. s, 1H), 5.33 (s, 2H), 7.17-7.26 (m, 2H), 7.32-7.40 (in, 2H), 7.48 (t, 1H), 7.52-7.61 (m, 1H), 7.66 (d, 1H), 8.36 (d, 1H).

### Example 47

### ent-N-(2-Amino-2-methylbutyl)-8-[(2,6-difluorobenzyl)oxy]-2-methylquinoline-4-carboxamide (Enantiomer B)

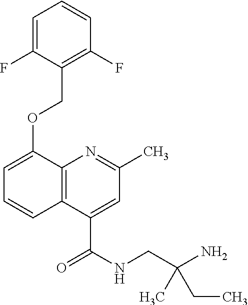 [SMILES: CCC(C)(N)CNC(=O)c1cc(C)nc2c(OCc3c(F)cccc3F)cccc12]

54 mg (0.082 mmol) of ent-benzyl {1-[({8-[(2,6-difluorobenzyl)oxy]-2-methylquinolin-4-yl}carbonyl)amino]-2-methylbutan-2-yl}carbamate trifluoroacetate (Enantiomer B) from Example 45A were dissolved in 2.1 ml of ethanol, 31 μl of TFA and 2.6 mg of palladium on activated carbon (10%) were added and the mixture was hydrogenated at atmospheric pressure for 4 hours. The reaction mixture was filtered and the filtrate was concentrated. The residue was dissolved in 2.1 ml of ethanol, 31 μl of TFA and 3 mg of palladium on activated carbon (10%) were added and the mixture was hydrogenated at atmospheric pressure for one hour. The reaction mixture was filtered and the filtrate was concentrated. The residue was dissolved in 2.1 ml of ethanol, 31 μl of TFA and 5 mg of palladium on activated carbon (10%) were added and the mixture was hydrogenated at atmospheric pressure for 2 hours. The reaction solution was filtered through a Millipore filter and the filtrate was concentrated. The residue was purified by preparative HPLC (RP18 column, mobile phase: acetonitrile/water gradient with addition of 0.1% TFA). The product fractions were combined and concentrated. Subsequently, the residue was taken up in dichloromethane and washed twice with saturated aqueous sodium bicarbonate solution. The combined aqueous phases were reextracted twice with dichloromethane. The combined organic phases were dried over sodium sulfate, filtered and concentrated. This gave 20 mg of the target compound (58% of theory).

LC-MS (Method 5): Rₜ=0.60 min

MS (ESpos): m/z=414 (M+H)⁺

¹H-NMR (400 MHz, DMSO-dₛ): δ [ppm]=0.89 (t, 3H), 0.99 (s, 3H), 1.32-1.41 (m, 2H), 1.47 (br. s, 2H), 2.64 (s, 3H), 3.24 (d, 2H), 5.33 (s, 2H), 7.16-7.26 (m, 2H), 7.37 (d, 1H), 7.44 (s, 1H), 7.48 (t, 1H), 7.52-7.65 (m, 2H), 8.49 (t, 1H).

### Example 48

### ent-8-[(2,6-Difluorobenzyl)oxy]-N-[(2S)-1-hydroxy-2-(5-methyl-1,3,4-thiadiazol-2-yl)propan-2-yl]-6-methylquinoline-4-carboxamide

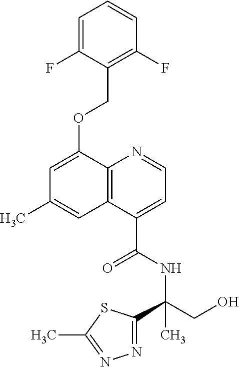 [SMILES: Cc1cc(OCc2c(F)cccc2F)c2nccc(C(=O)N[C@@](C)(CO)c3nnc(C)s3)c2c1]

51 mg (0.13 mmol) of HATU and 169 μl (0.97 mmol) of N,N-diisopropylethylamine were added to 40 mg (0.12 mmol) of 8-[(2,6-difluorobenzyl)oxy]-6-methylquinoline-4-carboxylic acid from Example 38A in 0.94 ml of DMF, and the mixture was stirred at room temperature for 10 min. 140 mg (0.49 mmol) of (2S)-2-amino-2-(5-methyl-1,3,4-thiadiazol-2-yl)propan-1-ol trifluoroacetate (preparable analogously to intermediate 307 in WO2014/084312) were then added, and the mixture was stirred at 60° C. for one hour. Acetonitrile, water and TFA were added and the reaction solution was purified by preparative HPLC (RP18 column, mobile phase: acetonitrile/water gradient with addition of 0.1% TFA). The product-containing fractions were combined and concentrated. The residue was taken up in dichloromethane and washed twice with saturated aqueous sodium bicarbonate solution. The combined aqueous phases were reextracted twice with dichloromethane. The combined organic phases were dried over sodium sulfate, filtered and concentrated. This gave 31 mg of the title compound (52% of theory; purity 98%).

LC-MS (Method 5): Rₜ=0.82 min

MS (ESpos): m/z=485 (M+H)⁺

¹H-NMR (400 MHz, DMSO-dₛ): δ [ppm]=1.80 (s, 3H), 2.73 (s, 3H), 3.82-3.90 (m, 1H), 3.93-4.00 (m, 1H), 5.29-5.35 (m, 3H), 5.33 (s, 2H), 7.19-7.27 (m, 2H), 7.32 (s, 1H), 7.49 (t, 2H), 7.53-7.63 (m, 1H), 8.79 (d, 1H), 9.06 (s, 1H) [further signal under solvent peak].

### Example 49

### rac-8-[(2,6-Difluorobenzyl)oxy]-N-{2-[2-(difluoromethyl)-2H-tetrazol-5-yl]-1-hydroxypropan-2-yl}-6-methylquinoline-4-carboxamide

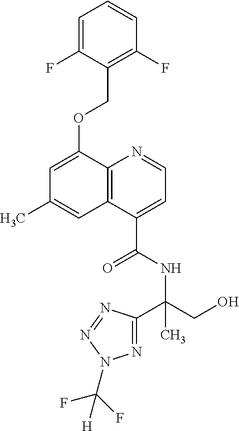 [SMILES: Cc1cc(OCc2c(F)cccc2F)c2nccc(C(=O)NC(C)(CO)c3nnn(C(F)F)n3)c2c1]

48 mg (0.13 mmol) of HATU and 106 μl (0.61 mmol) of N,N-diisopropylethylamine were added to 40 mg (0.12 mmol) of 8-[(2,6-difluorobenzyl)oxy]-6-methylquinoline-4-carboxylic acid from Example 38A in 0.94 ml of DMF, and the mixture was stirred at room temperature for 10 min. 26 mg (0.13 mmol) of rac-2-amino-2[2-(difluoromethyl)-2H-tetrazol-5-yl]propan-1-ol were then added, and the mixture was stirred at 60° C. for 2 hours. Acetonitrile, water and TFA were added and the reaction solution was purified by preparative HPLC (RP18 column, mobile phase: acetonitrile/water gradient with addition of 0.1% TFA). The product-containing fractions were combined and concentrated. The residue was taken up in dichloromethane and washed twice with saturated aqueous sodium bicarbonate solution. The combined aqueous phases were reextracted twice with dichloromethane. The combined organic phases were dried over sodium sulfate, filtered and concentrated. This gave 39 mg of the title compound (62% of theory; purity 98%).

LC-MS (Method 5): Rₜ=0.91 min

MS (ESpos): m/z=505 (M+H)⁺

¹H-NMR (400 MHz, DMSO-d₆): δ [ppm]=1.84 (s, 3H), 3.81-3.87 (m, 1H), 3.93-4.00 (m, 1H), 5.21 (t, 1H), 5.31 (s, 2H), 7.19-7.27 (m, 2H), 7.31 (s, 1H), 7.46-7.51 (m, 2H), 7.53-7.63 (m, 1H), 8.49-8.77 (t, 1H), 8.78 (s, 1H), 9.12 (s, 1H) [further signal under solvent peak].

### B. ASSESSMENT OF PHARMACOLOGICAL EFFICACY

The following abbreviations are used:

ATP adenosine triphosphate

Brij35 polyoxyethylene(23) lauryl ether

BSA bovine serum albumin

DTT dithiothreitol

TEA triethanolamine

The pharmacological action of the compounds of the invention can be demonstrated in the following assays:

B-1. Measurement of sGC Enzyme Activity by Means of PPi Detection

Soluble guanylyl cyclase (sGC) converts GTP to cGMP and pyrophosphate (PPi) when stimulated. PPi is detected with the aid of the method described in WO 2008/061626. The signal that arises in the assay increases as the reaction progresses and serves as a measure of the sGC enzyme activity. With the aid of a PPi reference curve, the enzyme can be characterized in a known manner, for example in terms of conversion rate, stimulability or Michaelis constant.

Conduct of the Test

To conduct the test, 29 μl of enzyme solution (0-10 nM soluble guanylyl cyclase (prepared according to Honicka et al., Journal of Molecular Medicine 77 (1999) 14-23), in 50 mM TEA, 2 mM magnesium chloride, 0.1% BSA (fraction V), 0.005% Brij 35, pH 7.5) were initially charged in the microplate, and 1 μl of the stimulator solution (0-10 μM 3-morpholinosydnonimine, SIN-1, Merck in DMSO) was added. The microplate was incubated at RT for 10 min. Then 20 μl of detection mix (1.2 nM Firefly Luciferase (Photinus pyralis luciferase, Promega), 29 μM dehydroluciferin (prepared according to Bitler & McElroy, Arch. Biochem. Biophys. 72 (1957) 358), 122 μM luciferin (Promega), 153 μM ATP (Sigma) and 0.4 mM DTT (Sigma) in 50 mM TEA, 2 mM magnesium chloride, 0.1% BSA (fraction V), 0.005% Brij 35, pH 7.5) were added. The enzyme reaction was started by adding 20 μl of substrate solution (1.25 mM guanosine 5′-triphosphate (Sigma) in 50 mM TEA, 2 mM magnesium chloride, 0.1% BSA (fraction V), 0.005% Brij 35, pH 7.5) and analyzed continuously in a luminometer.

B-2. Effect on a Recombinant Guanylate Cyclase Reporter Cell Line

The cellular activity of the compounds according to the invention is determined using a recombinant guanylate cyclase reporter cell line, as described in F. Wunder et al., Anal. Biochem. 339, 104-112 (2005).

Representative MEC values (MEC=minimum effective concentration) for the compounds of the invention are shown in the table below (in some cases as mean values from individual determinations):

TABLE A

| Example | MEC [μM] |
|---|---|
| 1 | 3 |
| 2 | 10 |
| 3 | 1 |
| 4 | 3 |
| 5 | 3 |
| 6 | 0.3 |
| 7 | 2 |
| 8 | 3 |
| 9 | 3 |
| 10 | 10 |
| 11 | 3 |
| 12 | 10 |
| 13 | 10 |
| 14 | 10 |
| 15 | 10 |
| 16 | 10 |
| 17 | 10 |
| 18 | 3 |
| 19 | 3 |
| 20 | 3 |
| 21 | 3 |
| 22 | 3 |
| 23 | 3 |
| 24 | 10 |
| 25 | 10 |
| 26 | 3 |
| 27 | 1 |
| 28 | 3 |
| 29 | 0.3 |
| 30 | 10 |
| 31 | 3 |
| 32 | 2 |
| 33 | 2 |
| 34 | 1 |
| 35 | 3 |
| 36 | 3 |
| 37 | 10 |
| 38 | 3 |
| 39 | 1 |
| 40 | 10 |
| 41 | 10 |
| 42 | 10 |
| 43 | 1 |
| 44 | 10 |
| 45 | 0.65 |
| 46 | 2 |
| 47 | 3 |
| 48 | 3 |
| 49 | 2 |

B-3. Vasorelaxant Effect In Vitro

Rabbits are stunned by a blow to the neck and exsanguinated. The aorta is removed, freed from adhering tissue and divided into rings of width 1.5 mm, which are placed individually under prestress into 5 ml organ baths with carbogen-sparged Krebs-Henseleit solution at 37° C. having the following composition (each in mM): sodium chloride: 119; potassium chloride: 4.8; calcium chloride dihydrate: 1; magnesium sulfate heptahydrate: 1.4; potassium dihydrogenphosphate: 1.2; sodium bicarbonate: 25; glucose: 10. The contractile force is determined with Statham UC2 cells, amplified and digitalized using A/D transducers (DAS-1802 HC, Keithley Instruments Munich), and recorded in parallel on linear recorders. To generate a contraction, phenylephrine is added to the bath cumulatively in increasing concentration. After several control cycles, the substance to be studied is added in increasing dosage each time in every further run, and the magnitude of the contraction is compared with the magnitude of the contraction attained in the last preceding run. This is used to calculate the concentration needed to reduce the magnitude of the control value by 50% (IC₅₀ value). The standard administration volume is 5 μl; the DMSO content in the bath solution corresponds to 0.1%.

B-4. Blood Pressure Measurement on Anesthetized Rats

Male Wistar rats having a body weight of 300-350 g are anesthetized with thiopental (100 mg/kg i.p.). After tracheotomy, a catheter is introduced into the femoral artery to measure the blood pressure. The substances to be tested are administered as solutions, either orally by means of a gavage or intravenously via the femoral vein (Stasch et al. Br. J. Pharmacol. 2002; 135: 344-355).

B-5. Radiotelemetry Measurement of Blood Pressure in Conscious, Spontaneously Hypertensive Rats

A commercially available telemetry system from DATA SCIENCES INTERNATIONAL DSI, USA, is employed for the blood pressure measurement on conscious rats described below.

The system consists of 3 main components:

implantable transmitters (Physiotel® telemetry transmitter)

receivers (Physiotel® receiver) which are linked via a multiplexer (DSI Data Exchange Matrix) to a

data acquisition computer.

The telemetry system makes it possible to continuously record blood pressure, heart rate and body motion of conscious animals in their usual habitat.

Animal Material

The studies are conducted on adult female spontaneously hypertensive rats (SHR Okamoto) with a body weight of >200 g. SHR/NCrl from the Okamoto Kyoto School of Medicine, 1963, were a cross of male Wistar Kyoto rats having greatly elevated blood pressure and female rats having slightly elevated blood pressure, and were handed over at F13 to the U.S. National Institutes of Health.

After transmitter implantation, the experimental animals are housed singly in type 3 Makrolon cages. They have free access to standard feed and water.

The day/night rhythm in the experimental laboratory is changed by the room lighting at 6:00 am and at 7:00 μm.

Transmitter Implantation

The TA1 1 PA—C40 telemetry transmitters used are surgically implanted under aseptic conditions in the experimental animals at least 14 days before the first experimental use. The animals instrumented in this way can be used repeatedly after the wound has healed and the implant has settled.

For the implantation, the fasted animals are anesthetized with pentobarbital (Nembutal, Sanofi: 50 mg/kg i.p.) and shaved and disinfected over a large area of their abdomens. After the abdominal cavity has been opened along the linea alba, the liquid-filled measuring catheter of the system is inserted into the descending aorta in the cranial direction above the bifurcation and fixed with tissue glue (VetBonD TM, 3M). The transmitter housing is fixed intraperitoneally to the abdominal wall muscle, and the wound is closed layer by layer.

An antibiotic (Tardomyocel COMP, Bayer, 1 ml/kg s.c.) is administered postoperatively for prophylaxis of infection.

Substances and Solutions

Unless stated otherwise, the substances to be studied are administered orally by gavage to a group of animals in each case (n=6). In accordance with an administration volume of 5 ml/kg of body weight, the test substances are dissolved in suitable solvent mixtures or suspended in 0.5% tylose.

A solvent-treated group of animals is used as control.

Experimental Procedure

The telemetry measuring unit present is configured for 24 animals. Each experiment is recorded under an experiment number (Vyear month day).

Each of the instrumented rats living in the system is assigned a separate receiving antenna (1010 Receiver, DSI).

The implanted transmitters can be activated externally by means of an incorporated magnetic switch. They are switched to transmission in the run-up to the experiment. The signals emitted can be detected online by a data acquisition system (Dataquest TM A.R.T. for WINDOWS, DSI) and processed accordingly. The data are stored in each case in a file created for this purpose and bearing the experiment number.

In the standard procedure, the following are measured for 10-second periods in each case:

systolic blood pressure (SBP)

diastolic blood pressure (DBP)

mean arterial pressure (MAP)

heart rate (HR)

activity (ACT).

The acquisition of measurements is repeated under computer control at 5-minute intervals. The source data obtained as absolute values are corrected in the diagram with the currently measured barometric pressure (Ambient Pressure Reference Monitor; APR-1) and stored as individual data. Further technical details are given in the extensive documentation from the manufacturer company (DSI).

Unless indicated otherwise, the test substances are administered at 9:00 am on the day of the experiment. Following the administration, the parameters described above are measured over 24 hours.

Evaluation

After the end of the experiment, the acquired individual data are sorted using the analysis software (DATAQUEST TM A.R.T. TM ANALYSIS). The blank value is assumed here to be the time 2 hours before administration, and so the selected data set encompasses the period from 7:00 am on the day of the experiment to 9:00 am on the following day.

The data are smoothed over a predefinable period by determination of the average (15-minute average) and transferred as a text file to a storage medium. The measured values presorted and compressed in this way are transferred to Excel templates and tabulated. For each day of the experiment, the data obtained are stored in a dedicated file bearing the number of the experiment. Results and test protocols are stored in files in paper form sorted by numbers.

Literature:

Klaus Witte, Kai Hu, Johanna Swiatek, Claudia Müssig, Georg Ertl and Björn Lemmer: Experimental heart failure in rats: effects on cardiovascular circadian rhythms and on myocardial β-adrenergic signaling. Cardiovasc Res 47 (2): 203-405, 2000; Kozo Okamoto: Spontaneous hypertension in rats. Int Rev Exp Pathol 7: 227-270, 1969; Maarten van den Buuse: Circadian Rhythms of Blood Pressure, Heart Rate, and Locomotor Activity in Spontaneously Hypertensive Rats as Measured With Radio-Telemetry. Physiology & Behavior 55(4): 783-787, 1994.

B-6. Determination of Pharmacokinetic Parameters Following Intravenous and Oral Administration

The pharmacokinetic parameters of the compounds according to the invention are determined in male CD-1 mice, male Wistar rats and female beagles. Intravenous administration in the case of mice and rats is carried out by means of a species-specific plasma/DMSO formulation, and in the case of dogs by means of a water/PEG400/ethanol formulation. In all species, oral administration of the dissolved substance is performed via gavage, based on a water/PEG400/ethanol formulation. The removal of blood from rats is simplified by inserting a silicone catheter into the right Vena jugularis externa prior to substance administration. The operation is carried out at least one day prior to the experiment with isofluran anesthesia and administration of an analgesic (atropine/rimadyl (3/1) 0.1 ml s.c.). The blood is taken (generally more than 10 time points) within a time window including terminal time points of at least 24 to a maximum of 72 hours after substance administration. The blood is removed into heparinized tubes. The blood plasma is then obtained by centrifugation; if required, it is stored at −20° C. until further processing.

An internal standard (which may also be a chemically unrelated substance) is added to the samples of the compounds of the invention, calibration samples and qualifiers, and there follows protein precipitation by means of acetonitrile in excess. Addition of a buffer solution matched to the LC conditions, and subsequent vortexing, is followed by centrifugation at 1000 g. The supernatant is analyzed by LC-MS/MS using C18 reversed-phase columns and variable mobile phase mixtures. The substances are quantified via the peak heights or areas from extracted ion chromatograms of specific selected ion monitoring experiments.

The plasma concentration/time plots determined are used to calculate the pharmacokinetic parameters such as AUC, Cₘₐₓ, t_{1/2} (terminal half-life), F (bioavailability), MRT (mean residence time) and CL (clearance), by means of a validated pharmacokinetic calculation program.

Since the substance quantification is performed in plasma, it is necessary to determine the blood/plasma distribution of the substance in order to be able to adjust the pharmacokinetic parameters correspondingly. For this purpose, a defined amount of substance is incubated in heparinized whole blood of the species in question in a rocking roller mixer for 20 min. After centrifugation at 1000 g, the plasma concentration is measured (by means of LC-MS/MS; see above) and determined by calculating the ratio of the C_{blood}/Cₚₗₐₛₘₐ value.

B-7. Metabolic Study

To determine the metabolic profile of the compounds of the invention, they are incubated with recombinant human cytochrome P450 (CYP) enzymes, liver microsomes or primary fresh hepatocytes from various animal species (e.g. rats, dogs), and also of human origin, in order to obtain and to compare information about a very substantially complete hepatic phase I and phase II metabolism, and about the enzymes involved in the metabolism.

The compounds of the invention were incubated with a concentration of about 0.1-10 μM. To this end, stock solutions of the compounds of the invention having a concentration of 0.01-1 mM in acetonitrile were prepared, and then pipetted with a 1:100 dilution into the incubation mixture. The liver microsomes and recombinant enzymes were incubated at 37° C. in 50 mM potassium phosphate buffer pH 7.4 with and without NADPH-generating system consisting of 1 mM NADP⁺, 10 mM glucose-6-phosphate and 1 unit glucose-6-phosphate dehydrogenase. Primary hepatocytes were incubated in suspension in Williams E medium, likewise at 37° C. After an incubation time of 0-4 h, the incubation mixtures were stopped with acetonitrile (final concentration about 30%) and the protein was centrifuged off at about 15 000×g. The samples thus stopped were either analyzed directly or stored at −20° C. until analysis.

The analysis is carried out by high-performance liquid chromatography with ultraviolet and mass spectrometry detection (HPLC-UV-MS/MS). To this end, the supernatants of the incubation samples are chromatographed with suitable C18 reversed-phase columns and variable mobile phase mixtures of acetonitrile and 10 mM aqueous ammonium formate solution or 0.05% formic acid. The UV chromatograms in conjunction with mass spectrometry data serve for identification, structural elucidation and quantitative estimation of the metabolites, and for quantitative metabolic reduction of the compound of the invention in the incubation mixtures.

B-8. Caco-2 Permeability Test

The permeability of a test substance was determined with the aid of the Caco-2 cell line, an established in vitro model for permeability prediction at the gastrointestinal barrier (Artursson, P. and Karlsson, J. (1991). Correlation between oral drug absorption in humans and apparent drug permeability coefficients in human intestinal epithelial (Caco-2) cells. Biochem. Biophys.175 (3), 880-885). The Caco-2 cells (ACC No. 169, DSMZ, Deutsche Sammlung von Mikroorganismen and Zellkulturen, Braunschweig, Germany) were sown in 24-well plates having an insert and cultivated for 14 to 16 days. For the permeability studies, the test substance was dissolved in DMSO and diluted to the final test concentration with transport buffer (Hanks Buffered Salt Solution, Gibco/Invitrogen, with 19.9 mM glucose and 9.8 mM HEPES). In order to determine the apical to basolateral permeability (PₐₚₚA-B) of the test substance, the solution comprising the test substance was applied to the apical side of the Caco-2 cell monolayer, and transport buffer to the basolateral side. In order to determine the basolateral to apical permeability (PₐₚₚB-A) of the test substance, the solution comprising the test substance was applied to the basolateral side of the Caco-2 cell monolayer, and transport buffer to the apical side. At the start of the experiment, samples were taken from the respective donor compartment in order to ensure the mass balance. After an incubation time of two hours at 37° C., samples were taken from the two compartments. The samples were analyzed by means of LC-MS/MS and the apparent permeability coefficients (Pₐₚₚ) were calculated. For each cell monolayer, the permeability of Lucifer Yellow was determined to ensure cell layer integrity. In each test run, the permeability of atenolol (marker for low permeability) and sulfasalazine (marker for active excretion) was also determined as quality control.

B-9. hERG Potassium Current Assay

The hERG (human ether-a-go-go related gene) potassium current makes a significant contribution to the repolarization of the human cardiac action potential (Scheel et al., 2011). Inhibition of this current by pharmaceuticals can in rare cases cause potentially lethal cardiac arrhythmias, and is therefore studied at an early stage during drug development.

The functional hERG assay used here is based on a recombinant HEK293 cell line which stably expresses the KCNH2(HERG) gene (Zhou et al., 1998). These cells are studied by means of the “whole-cell voltage-clamp” technique (Hamill et al., 1981) in an automated system (Patchliner™; Nanion, Munich, Germany), which controls the membrane voltage and measures the hERG potassium current at room temperature. The PatchControlHT™ software (Nanion) controls the Patchliner system, data capture and data analysis. The voltage is controlled by 2 EPC-10 quadro amplifiers controlled by the PatchMasterPro™ software (both: HEKA Elektronik, Lambrecht, Germany). NPC-16 chips with moderate resistance (˜2MΩ; Nanion) serve as the planar substrate for the voltage clamp experiments.

NPC-16 chips are filled with intra- and extracellular solution (cf. Himmel, 2007) and with cell suspension. After forming a gigaohm seal and establishing whole-cell mode (including several automated quality control steps), the cell membrane is clamped at the −80 mV holding potential. The subsequent voltage clamp protocol changes the command voltage to +20 mV (for 1000 ms), −120 mV (for 500 ms), and back to the −80 mV holding potential; this is repeated every 12 s. After an initial stabilization phase (about 5-6 minutes), test substance solution is introduced by pipette in rising concentrations (e.g. 0.1, 1, and 10 μmol/l) (exposure about 5-6 minutes per concentration), followed by several washing steps.

The amplitude of the inward “tail” current which is generated by a change in potential from +20 mV to −120 mV serves to quantify the hERG potassium current, and is described as a function of time (IgorPro™ Software). The current amplitude at the end of various time intervals (for example stabilization phase before test substance, first/second/third concentration of test substance) serves to establish a concentration/effect curve, from which the half-maximum inhibiting concentration IC₅₀ of the test substance is calculated.
Hamill O P, Marty A, Neher E, Sakmann B, Sigworth F J. Improved patch-clamp techniques for high-resolution current recording from cells and cell-free membrane patches. Pfluegers Arch 1981; 391:85-100.
Himmel H M. Suitability of commonly used excipients for electrophysiological in-vitro safety pharmacology assessment of effects on hERG potassium current and on rabbit Purkinje fiber action potential. J Pharmacol Toxicol Methods 2007; 56:145-158.
Scheel O, Himmel H, Rascher-Eggstein G, Knott T. Introduction of a modular automated voltage-clamp platform and its correlation with manual human ether-a-go-go related gene voltage-clamp data. Assay Drug Dev Technol 2011; 9:600-607.
Zhou Z F, Gong Q, Ye B, Fan Z, Makielski J C, Robertson G A, January C T. Properties of hERG channels stably expressed in HEK293 cells studied at physiological temperature. Biophys J 1998; 74:230-241.

### C. WORKING EXAMPLES OF PHARMACEUTICAL COMPOSITIONS

The compounds of the invention can be converted to pharmaceutical preparations as follows:

Tablet:

Composition:

100 mg of the compound of the invention, 50 mg of lactose (monohydrate), 50 mg of corn starch (native), 10 mg of polyvinylpyrrolidone (PVP 25) (BASF, Ludwigshafen, Germany) and 2 mg of magnesium stearate.

Tablet weight 212 mg. Diameter 8 mm, radius of curvature 12 mm.

Production:

The mixture of compound of the invention, lactose and starch is granulated with a 5% solution (w/w) of the PVP in water. The granules are dried and then mixed with the magnesium stearate for 5 minutes. This mixture is compressed using a conventional tableting press (see above for format of the tablet). The guide value used for the pressing is a pressing force of 15 kN.

Suspension for Oral Administration:

Composition:

1000 mg of the compound of the invention, 1000 mg of ethanol (96%), 400 mg of Rhodigel® (xanthan gum from FMC, Pennsylvania, USA) and 99 g of water.

10 ml of oral suspension correspond to a single dose of 100 mg of the compound of the invention.

Production:

The Rhodigel is suspended in ethanol; the compound of the invention is added to the suspension. The water is added while stirring. The mixture is stirred for about 6 h until the swelling of the Rhodigel is complete.

Solution for Oral Administration:

Composition:

500 mg of the compound of the invention, 2.5 g of polysorbate and 97 g of polyethylene glycol 400. 20 g of oral solution correspond to a single dose of 100 mg of the compound of the invention.

Production:

The compound of the invention is suspended in the mixture of polyethylene glycol and polysorbate with stirring. The stirring operation is continued until dissolution of the compound of the invention is complete.

I.V. Solution:

The compound of the invention is dissolved in a concentration below the saturation solubility in a physiologically acceptable solvent (e.g. isotonic saline solution, glucose solution 5% and/or PEG 400 solution 30%). The resulting solution is subjected to sterile filtration and dispensed into sterile and pyrogen-free injection vessels.

1. A compound of the formula (I-A) or (I-B) 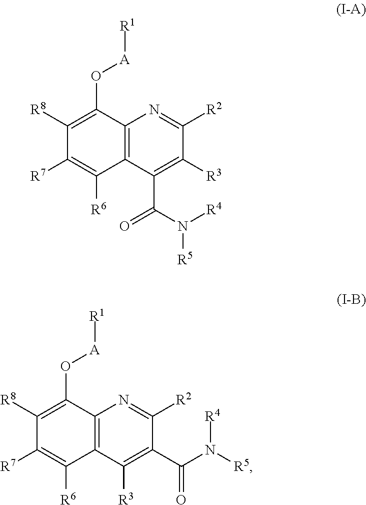
in which
A represents CH₂, CD₂ or CH(CH₃),
R¹ represents (C₄-C₆)-alkyl, (C₃-C₇)-cycloalkyl, pyridyl or phenyl,
where (C₄-C₆)-alkyl may be up to hexasubstituted by fluorine,
where (C₃-C₇)-cycloalkyl may be substituted by 1 to 4 substituents independently of one another selected from the group consisting of fluorine, trifluoromethyl and (C₁-C₄)-alkyl,
where pyridyl is substituted by 1 or 2 substituents independently of one another selected from the group consisting of halogen, cyano and (C₁-C₄)-alkyl,
and
where phenyl may be substituted by 1 to 4 substituents independently of one another selected from the group consisting of halogen, cyano, monofluoromethyl, difluoromethyl, trifluoromethyl, (C₁-C₄)-alkyl, (C₂-C₃)-alkynyl, (C₁-04-alkoxy, (C₃-C₅)-cyclopropyl, difluoromethoxy and trifluoromethoxy,
R² represents hydrogen, halogen, (C₁-C₄)-alkyl, (C₁-C₄)-alkoxy, cyclopropyl, monofluoromethyl, difluoromethyl or trifluoromethyl,
R³ represents hydrogen, halogen, (C₁-C4-alkyl, (C₁-C₄)-alkoxy, cyclopropyl, monofluoromethyl, difluoromethyl or trifluoromethyl,
R⁴ represents hydrogen or (C₁-C₄)-alkyl,
R⁵ is a group of the formula 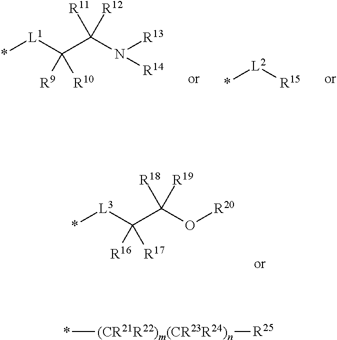
where
* represents the point of attachment to the amino group,
L¹ represents a bond, methanediyl or 1,2-ethanediyl,
in which methanediyl and 1,2-ethanediyl may be substituted by 1 or 2 substituents independently of one another selected from the group consisting of fluorine, trifluoromethyl, (C₁-C₄)-alkyl, (C₃-C₅)-cycloalkyl, hydroxy and (C₁-C₄)-alkoxy,
L²represents a bond or (C₁-C₄)-alkanediyl,
in which (C₁-C₄)-alkanediyl may be substituted by 1 to 3 substituents independently of one another selected from the group consisting of fluorine, trifluoromethyl, (C₁-C₄)-alkyl, (C₃-C₅)-cycloalkyl, hydroxy and (C₁-C₄)-alkoxy,
L³ is a bond, methanediyl or 1,2-ethanediyl,
in which methanediyl or 1,2-ethanediyl may be substituted by 1 or 2 substituents independently of one another selected from the group consisting of fluorine, trifluoromethyl, (C₁-C₄)-alkyl, (C₃-C₇)-cycloalkyl, hydroxy and (C₁-C₄)-alkoxy,
R⁹ represents hydrogen, (C₁-C₄)-alkyl, (C₂-C₆)-alkenyl, (C₂-C₆)-alkynyl, (C₃-C₇)-cycloalkyl, 5- or 6-membered heteroaryl or phenyl,
in which (C₁-C₄)-alkyl may be substituted by 1 or 2 substituents independently of one another selected from the group consisting of trifluoromethyl, difluoromethoxy, trifluoromethoxy, hydroxy, (C₁-C₄)-alkoxy, (C₁-C₄)-alkoxycarbonyl, (C₁-C₄)-alkylthio, (C₁-C₄)-alkylsulfonyl, phenyl, phenoxy and benzyloxy, and up to hexasubstituted by fluorine,
in which phenyl, phenoxy and benzyloxy may be substituted by 1 to 3 halogen substituents,
in which (C₃-C₇)-cycloalkyl may be substituted by 1 or 2 substituents independently of one another selected from the group consisting of fluorine, trifluoromethyl, (C₁-C₄)-alkyl and (C₁-C₄)-alkoxy,
and
in which phenyl and 5- or 6-membered heteroaryl may be substituted by 1 to 3 substituents independently of one another selected from the group consisting of halogen, cyano, nitro, trifluoromethyl, (C₁-C₄)-alkyl, (C₁-C₄)-alkoxy, difluoromethoxy, trifluoromethoxy and (C₁-C₄)-alkylsulfonyl,
R¹⁰ represents hydrogen or (C₁-C₆)-alkyl,
or
R⁹ and R¹⁰ together with the carbon atom to which they are attached form a 3- to 7-membered carbocycle or a 4- to 7-membered heterocycle,
in which the 3- to 7-membered carbocycle and the 4- to 7-membered heterocycle for their part may be substituted by 1 or 2 substituents independently of one another selected from the group consisting of fluorine and (C₁-C₄)-alkyl,
R¹¹ represents hydrogen, (C₁-C₁₀)-alkyl, (C₃-C₇)-cycloalkyl, (C₂-C₆)-alkenyl, (C₂-C₆)-alkynyl, 5- or 6-membered heteroaryl or phenyl,
in which (C₁-C₁₀)-alkyl may be substituted by 1 or 2 substituents independently of one another selected from the group consisting of trifluoromethyl, difluoromethoxy, trifluoromethoxy, hydroxy, (C₁-C₄)-alkoxy, benzyloxy, phenoxy and phenyl, and up to hexasubstituted by fluorine,
in which benzyloxy, phenoxy and phenyl may be substituted by 1 to 3 halogen or (C₁-C₄)-alkoxy substituents,
in which (C₃-C₇)-cycloalkyl may be substituted by 1 or 2 fluorine or (C₁-C₄)-alkyl substituents,
and
in which phenyl and 5- or 6-membered heteroaryl may be substituted by 1 to 3 substituents independently of one another selected from the group consisting of halogen, cyano, trifluoromethyl, (C₁-C₄)-alkyl, (C₁-C₄)-alkoxy and (C₁-C₄)-alkylsulfonyl,
R¹² represents hydrogen or (C₁-C₆)-alkyl,
or
R¹¹ and R¹² together with the carbon atom to which they are attached form a 3- to 7-membered carbocycle or a 4- to 7-membered heterocycle,
in which the 3- to 7-membered carbocycle and the 4- to 7-membered heterocycle for their part may be substituted by 1 or 2 substituents independently of one another selected from the group consisting of fluorine and (C₁-C₄)-alkyl,
or
R⁹ and R¹¹ together with the carbon atoms to which they are attached form a 3- to 7-membered carbocycle or a 4- to 7-membered heterocycle,
with the proviso that not more than one of the R⁹ and R¹⁰, R¹¹ and R¹², and R⁹ and R¹¹ radical pairs at the same time forms a carbo- or heterocycle,
with the proviso that the R⁹ and R¹¹ radicals are not both simultaneously phenyl or 5- or 6-membered heteroaryl,
R¹³ represents hydrogen or (C₁-C₄)-alkyl,
in which (C₁-C₄)-alkyl may be substituted by 1 to 3 substituents independently of one another selected from the group consisting of fluorine, trifluoromethyl, hydroxy and (C₁-C₄)-alkoxy,
R¹⁴ represents hydrogen, (C₁-C₆)-alkyl, (C₃-C₇)-cycloalkyl, phenyl or benzyl,
in which (C₁-C₆)-alkyl may be substituted by 1 to 3 substituents independently of one another selected from the group consisting of fluorine, trifluoromethyl, hydroxy, (C₁-C₄)-alkoxy and phenoxy,
and
in which phenyl and benzyl may be substituted by 1 to 3 substituents independently of one another selected from the group consisting of halogen and trifluoromethyl,
or
R¹³ and R¹⁴ together with the nitrogen atom to which they are attached form a 4- to 7-membered azaheterocycle,
R¹⁵ represents 5- to 10-membered azaheterocyclyl attached via a ring carbon atom,
in which 5- to 10-membered azaheterocyclyl attached via a ring carbon atom may be substituted by 1 to 2 trifluoromethyl, (C₃-C₇)-cycloalkyl, oxo and benzyl substituents, and up to four times by (C₁-C₄)-alkyl and up to twice by fluorine,
in which 5- to 10-membered azaheterocyclyl may be fused to a phenyl ring or a pyridyl ring, which for their part may be substituted by 1 or 2 substituents independently of one another selected from the group consisting of halogen, (C₁-C₄)-alkyl, (C₁-C₄)-alkoxy and trifluoromethyl,
R¹⁶ represents hydrogen, (C₁-C₁₀)-alkyl, (C₂-C₆)-alkenyl, (C₂-C₆)-alkynyl, (C₃-C₇)-cycloalkyl, (C₁-C₄)-alkoxycarbonyl, —(C═O)NR²⁶R²⁷, 5- or 6-membered heteroaryl or phenyl,
in which (C₁-C₁₀)-alkyl may be substituted by 1 or 2 substituents independently of one another selected from the group consisting of difluoromethoxy, trifluoromethoxy, hydroxy, (C₁-C₄)-alkoxy, (C₁-C₄)-alkoxycarbonyl, (C₁-C₄)-alkylthio, (C₁-C₄)-alkylsulfonyl, phenyl, phenoxy and benzyloxy, and up to hexasubstituted by fluorine,
in which phenyl, phenoxy and benzyloxy for their part may be substituted by 1 to 3 halogen substituents,
in which (C₃-C₇)-cycloalkyl may be substituted by 1 or 2 substituents independently of one another selected from the group consisting of fluorine, trifluoromethyl, (C₁-C₄)-alkyl and (C₁-C₄)-alkoxy,
in which R²⁶ represents hydrogen, (C₁-C₄)-alkyl, (C₃-C₇)-cycloalkyl, aryl or naphthyl,
in which R²⁷ represents hydrogen or methyl,
and
in which phenyl and 5- or 6-membered heteroaryl may be substituted by 1 to 3 substituents independently of one another selected from the group consisting of halogen, cyano, trifluoromethyl, difluoromethyl, (C₁-C₄)-alkyl, (C₁-C₄)-alkoxy and (C₁-C₄)-alkylsulfonyl,
in which (C₁-C₄)-alkyl may be substituted by amino or hydroxy,
R¹⁷ represents hydrogen or (C₁-C₆)-alkyl,
in which (C₁-C₄)-alkyl may be substituted by hydroxy,
or
R¹⁶ and R⁷ together with the carbon atom to which they are attached form a 3- to 7-membered carbocycle or a 4- to 7-membered heterocycle,
in which the 3- to 7-membered carbocycle and the 4- to 7-membered heterocycle for their part may be substituted by 1 or 2 substituents independently of one another selected from the group consisting of fluorine and (C₁-C₄)-alkyl,
R¹⁸ represents hydrogen, (C₁-C₆)-alkyl, (C₂-C₆)-alkenyl, (C₂-C₆)-alkynyl, (C₃-C₇)-cycloalkyl, (C₁-C₄)-alkoxycarbonyl, 5- or 6-membered heteroaryl or phenyl,
in which (C₁-C₆)-alkyl may be substituted by 1 or 2 substituents independently of one another selected from the group consisting of trifluoromethyl, difluoromethoxy, trifluoromethoxy, hydroxy, (C₁-C₄)-alkoxy, (C₁-C₄)-alkoxycarbonyl, (C₁-C₄)-alkylthio, (C₁-C₄)-alkylsulfonyl, phenyl, phenoxy and benzyloxy, and up to hexasubstituted by fluorine,
in which phenyl, phenoxy and benzyloxy for their part may be substituted by 1 to 3 halogen substituents,
in which (C₃-C₇)-cycloalkyl may be substituted by 1 or 2 substituents independently of one another selected from the group consisting of fluorine, trifluoromethyl, (C₁-C₄)-alkyl and (C₁-C₄)-alkoxy,
and
in which phenyl and 5- or 6-membered heteroaryl may be substituted by 1 to 3 substituents independently of one another selected from the group consisting of halogen, cyano, trifluoromethyl, (C₁-C₄)-alkyl, (C₁-C₄)-alkoxy and (C₁-C₄)-alkylsulfonyl,
R¹⁹ represents hydrogen or (C₁-C₆)-alkyl,
in which (C₁-C₄)-alkyl may be substituted by hydroxy,
or
R¹⁸ and R¹⁹ together with the carbon atom to which they are attached form a 3- to 7-membered carbocycle or a 4- to 7-membered heterocycle,
in which the 3- to 7-membered carbocycle and the 4- to 7-membered heterocycle for their part may be substituted by 1 or 2 substituents independently of one another selected from the group consisting of fluorine and (C₁-C₄)-alkyl,
with the proviso that the R¹⁶ and R¹⁸ radicals are not both simultaneously phenyl or 5- or 6-membered heteroaryl,
or
R¹⁶ and R¹⁸ together with the carbon atoms to which they are attached form a 3- to 7-membered carbocycle or a 4- to 7-membered heterocycle,
with the proviso that not more than one of the R¹⁶ and R¹⁷, R¹⁸ and R¹⁹, and R¹⁶ and R¹⁸ radical pairs at the same time forms a carbo- or heterocycle,
R²⁰ represents hydrogen or (C₁-C₄)-alkyl,
in which (C₁-C₄)-alkyl may be substituted by 1 to 5 fluorine substituents,
m represents 0, 1 or 2,
n represents 0 or 1,
R²¹ represents hydrogen, cyano or (C₁-C₆)-alkyl,
in which (C₁-C₆)-alkyl may be substituted by 1 to 5 fluorine substituents,
R²² represents hydrogen or (C₁-C₄)-alkyl,
in which (C₁-C₄)-alkyl may be substituted by 1 to 5 fluorine substituents,
R²³ represents hydrogen or (C₁-C₆)-alkyl,
in which (C₁-C₆)-alkyl may be substituted by 1 to 5 fluorine substituents,
R²⁴ represents hydrogen or (C₁-C₄)-alkyl,
in which (C₁-C₄)-alkyl may be substituted by 1 to 5 fluorine substituents,
or
R²¹ and R²² together with the carbon atom to which they are attached form a 3- to 7-membered carbocycle or a 4- to 7-membered heterocycle,
in which the 3- to 7-membered carbocycle and the 4- to 7-membered heterocycle for their part may be substituted by 1 or 2 substituents independently of one another selected from the group consisting of fluorine and (C₁-C₄)-alkyl,
or
R²³ and R²⁴ together with the carbon atom to which they are attached form a 3- to 7-membered carbocycle or a 4- to 7-membered heterocycle,
in which the 3- to 7-membered carbocycle and the 4- to 7-membered heterocycle for their part may be substituted by 1 or 2 substituents independently of one another selected from the group consisting of fluorine and (C₁-C₄)-alkyl,
or
R²¹ and R²³ together with the carbon atom to which they are attached form a 3- to 7-membered carbocycle or a 4- to 7-membered heterocycle,
in which the 3- to 7-membered carbocycle and the 4- to 7-membered heterocycle for their part may be substituted by 1 or 2 substituents independently of one another selected from the group consisting of fluorine and (C₁-C₄)-alkyl,
with the proviso that not more than one of the R²¹ and R²², R²³ and R²⁴, and R²¹ and R²³ radical pairs at the same time forms a carbo- or heterocycle,
R²⁵ represents (C₁-C₆)-alkyl, (C₁-C₆)-alkoxy, (C₁-C₄)-alkoxycarbonyl, hydroxycarbonyl, aminocarbonyl, aminosulfonyl, 5- to 10-membered heterocyclyl attached via a ring carbon atom, 5- to 10-membered carbocyclyl, phenyl or 5- to 10-membered heteroaryl,
in which (C₁-C₆)-alkyl may be substituted by cyano and up to hexasubstituted by fluorine,
in which (C₁-C₆)-alkoxy may be substituted by hydroxy, amino, monoalkylamino, dialkylamino, cyclopropyl, phenyl or (C₂-C₄)-alkenyl,
in which aminocarbonyl may be substituted by (C₁-C₆)-alkyl or (C₃-C₆)-cycloalkyl,
in which aminosulfonyl may be substituted by (C₁-C₆)-alkyl or (C₃-C₆)-cycloalkyl,
in which phenyl may be substituted by 1 to 3 substituents independently of one another selected from the group consisting of halogen, cyano, trifluoromethyl, difluoromethyl, (C₁-C₆)-alkyl, (C₁-C₄)-alkylcarbonyl, (C₁-C₄)-alkoxycarbonyl, hydroxycarbonyl, —(C═O)NR²⁸R²⁹, (C₁-C₄)-alkylsulfonyl, (C₃-C₆)-cycloalkylsulfonyl, (C₁-C₄)-alkylthio, (C₁-C₄)-alkoxy, trifluoromethoxy, difluoromethoxy, phenoxy, hydroxyl, 5- to 10-membered heteroaryl, 4- to 7-membered heterocyclyl and (C₃-C₇)-cycloalkyl,
in which (C₁-C₆)-alkyl may be substituted by 1 or 2 substituents independently of one another selected from the group consisting of fluorine, trifluoromethoxy, (C₁-C₄)-alkylcarbonyl, —(C═O)NR²⁸R²⁹, (C₁-C₄)-alkoxy, (C₃-C₆)-cycloalkyl, morpholinyl, piperidinyl, pyrrolidinyl, piperazinyl, phenyl, hydroxy and amino,
in which phenyl may be substituted by 1 to 3 halogen substituents,
in which amino may be substituted by 1 or 2 substituents independently of one another selected from the group consisting of (C₁-C₆)-alkyl, (C₁-C₄)-alkylcarbonyl, (C₃-C₆)-cycloalkylsulfonyl, (C₁-C₄)-alkylsulfonyl and methoxy-(C₁-C₄)-alkyl,
in which (C₃-C₆)-cycloalkyl may be substituted by amino or hydroxy,
and in which
R²⁸ and R²⁹ each independently of one another represent hydrogen, (C₁-C₄)-alkyl or (C₃-C₇)-cycloalkyl,
in which 5- to 10-membered heteroaryl may be substituted by 1 to 3 substituents independently of one another selected from the group consisting of fluorine, chlorine, cyano, (C₁-C₆)-alkyl, trifluoromethyl, (C₁-C₄)-alkoxy, amino, (C₁-C₄)-alkoxycarbonyl, hydroxycarbonyl, —(C═O)NR²⁸R²⁹, phenyl, pyridyl, pyrimidyl, 1,3-thiazol-5-yl and (C₃-C₇)-cycloalkyl,
in which (C₁-C₆)-alkyl may be substituted by 1 to 3 substituents independently of one another selected from the group consisting of halogen, cyano, hydroxy, amino, trifluoromethyl, difluoromethyl, (C₁-C₄)-alkylsulfonyl, (C₁-C₄)-alkylcarbonyl, (C₁-C₄)-alkoxycarbonyl, hydroxycarbonyl, (C₁-C₄)-alkylthio, (C₁-C₄)-alkoxy, trifluoromethoxy, difluoromethoxy, phenoxy, phenyl, pyridyl, pyrimidyl, 5-membered heteroaryl, tetrahydrothiophenyl 1,1-dioxide, (C₃-C₇)-cycloalkyl, morpholinyl, piperidinyl, pyrrolidinyl, 2-oxopyrrolidin-1-yl, piperazinyl, tetrahydrothiophenyl 1,1-dioxide, thiomorpholinyl 1,1-dioxide and azetidine,
in which 5-membered heteroaryl may be substituted by 1 to 3 substituents independently of one another selected from the group consisting of halogen, (C₁-C₄)-alkyl and (C₁-C₄)-alkoxy,
in which piperidinyl may be substituted by 1 to 4 fluorine substituents,
in which phenyl may be substituted by 1 to 3 substituents independently of one another selected from the group consisting of halogen, (C₁-C₄)-alkyl and (C₁-C₄)-alkoxy,
in which azetidine may be substituted by hydroxy, in which piperazinyl may be substituted by 1 to 3 substituents independently of one another selected from the group consisting of (C₁-C₄)-alkyl, (C₃-C₇)-cycloalkyl and trifluoromethyl,
and in which
R²⁸ and R²⁹ each independently of one another represent hydrogen, (C₁-C₄)-alkyl or (C₃-C₇)-cycloalkyl,
in which 5- to 10-membered heterocyclyl attached via a ring carbon atom may be substituted by 1 to 3 substituents independently of one another selected from the group consisting of oxo, fluorine, trifluoromethyl, hydroxy and (C₁-C₄)-alkyl,
in which 5- to 10-membered heterocyclyl attached via a ring carbon atom may be fused to a phenyl ring or a pyridyl ring, which for their part may be substituted by 1 to 3 substituents selected from the group consisting of halogen, (C₁-C₄)-alkyl and trifluoromethyl,
and
in which 5- to 10-membered carbocyclyl may be substituted by 1 to 3 substituents independently of one another selected from the group consisting of trifluoromethyl, fluorine, cyano, hydroxy, hydroxycarbonyl, (C₁-C₄)-alkoxycarbonyl, amino and (C₁-C₄)-alkyl,
in which (C₁-C₄)-alkyl may be substituted by hydroxy or hydroxycarbonyl,
in which 5- to 10-membered carbocyclyl may be fused to a phenyl ring or a pyridyl ring, which for its part may be substituted by 1 to 3 substituents selected from the group consisting of halogen, (C₁-C₄)-alkyl, (C₁-C₄)-alkoxy and trifluoromethyl,
R⁶ represents hydrogen,
R⁷ represents hydrogen, halogen, cyano, difluoromethyl, trifluoromethyl, (C₁-C₄)-alkyl, (C₃-C₇)-cycloalkyl, (C₂-C₄)-alkynyl, (C₁-C₄)-alkylamino, difluoromethoxy, trifluoromethoxy, (C₁-C₄)-alkoxy, amino, 4- to 7-membered heterocyclyl or 5- or 6-membered heteroaryl,
R⁸ represents hydrogen, cyano or halogen,
and the N-oxides, salts, solvates, salts of the N-oxides and solvates of the N-oxides and salts thereof.

2. A compound of the formula (I-A) or (I-B) as claimed in claim 1 in which
A represents CH₂, CD₂ or CH(CH₃),
R¹ represents (C₃-C₇)-cycloalkyl, pyridyl or phenyl,
where pyridyl is substituted by 1 or 2 fluorine substituents,
and
where phenyl may be substituted by 1 to 4 substituents independently of one another selected from the group consisting of halogen, cyano, monofluoromethyl, difluoromethyl, trifluoromethyl, (C₁-C₄)-alkyl, (C₁-C₄)-alkoxy and (C₃-C₅)-cyclopropyl,
R² represents hydrogen, (C₁-C₄)-alkyl, cyclopropyl, difluoromethyl or trifluoromethyl,
R³ represents hydrogen, (C₁-C₄)-alkyl, cyclopropyl, difluoromethyl or trifluoromethyl,
R⁴ represents hydrogen or (C₁-C₄)-alkyl,
R⁵ represents a group of the formula 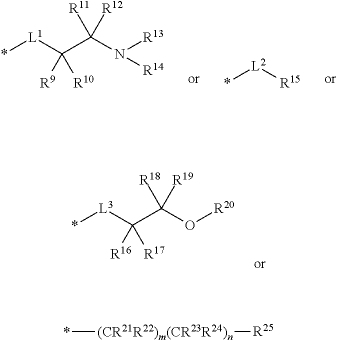
where
* represents the point of attachment to the amino group,
L¹ represents a bond, methanediyl or 1,2-ethanediyl,
L²represents a bond, methanediyl or 1,2-ethanediyl,
L³represents a bond, methanediyl or 1,2-ethanediyl,
R⁹ represents hydrogen, (C₁-C₆)-alkyl, (C₃-C₇)-cycloalkyl, 5- or 6-membered heteroaryl or phenyl,
in which (C₁-C₆)-alkyl may be substituted by 1 to 5 fluorine substituents,
and
in which phenyl and 5- or 6-membered heteroaryl may be substituted by 1 to 3 substituents independently of one another selected from the group consisting of fluorine, chlorine, bromine, cyano, trifluoromethyl, methyl, ethyl, methoxy or ethoxy,
R¹⁰ represents hydrogen or (C₁-C₄)-alkyl,
or
R⁹ and R¹⁹ together with the carbon atom to which they are attached form a 3- to 5-membered carbocycle,
R¹¹ represents hydrogen, (C₁-C₈)-alkyl, (C₃-C₅)-cycloalkyl, 5- or 6-membered heteroaryl or phenyl,
in which (C₁-C₈)-alkyl may be substituted by 1 to 5 fluorine substituents,
in which (C₁-C₈)-alkyl may be substituted by (C₁-C₄)-alkoxy, benzyloxy, phenoxy or phenyl,
in which benzyloxy, phenoxy and phenyl may be substituted by 1 to 3 substituents each independently selected from the group of fluorine, chlorine, bromine, methoxy and ethoxy,
in which (C₃-C₅)-cycloalkyl may be substituted by 1 or 2 fluorine or (C₁-C₄)-alkyl substituents,
and
in which phenyl and 5- or 6-membered heteroaryl may be substituted by 1 to 3 substituents independently of one another selected from the group consisting of fluorine, chlorine, bromine, cyano, trifluoromethyl, methyl, ethyl, methoxy or ethoxy,
R¹² represents hydrogen or (C₁-C₄)-alkyl,
or
R¹¹ and R¹² together with the carbon atom to which they are attached form a 3- to 5-membered carbocycle,
or
R⁹ and R¹¹ together with the carbon atoms to which they are attached form a 3- to 6-membered carbocycle or a 4- to 7-membered heterocycle,
with the proviso that not more than one of the R⁹ and R¹⁰, R¹¹ and R¹², and R⁹ and R¹¹ radical pairs at the same time forms a carbocycle,
and
with the proviso that the R⁹ and R¹¹ radicals are not both simultaneously phenyl or 5- or 6-membered heteroaryl,
R¹³ represents hydrogen or (C₁-C₄)-alkyl,
in which (C₁-C₄)-alkyl may be substituted by 1 to 5 fluorine substituents,
R¹⁴ represents hydrogen, (C₁-C₆)-alkyl or (C₃-C₇)-cycloalkyl,
in which (C₁-C₆)-alkyl may be substituted by 1 to 5 fluorine substituents,
or
R¹³ and R¹⁴ together with the nitrogen atom to which they are attached form a 4- to 7-membered azaheterocycle,
R¹⁵ represents 5- to 10-membered azaheterocyclyl attached via a ring carbon atom,
in which 5- to 10-membered azaheterocyclyl attached via a ring carbon atom may be substituted by 1 to 5 substituents independently of one another selected from the group consisting of fluorine, methyl and ethyl,
R¹⁶ represents hydrogen, (C₁-C₁₀)-alkyl, (C₃-C₅)-cycloalkyl, —(C═O)NR²⁶R²⁷, 5- or 6-membered heteroaryl or phenyl,
in which (C₁-C₁₀)-alkyl may be substituted by difluoromethoxy, trifluoromethoxy, hydroxy or (C₁-C₄)-alkoxy and up to hexasubstituted by fluorine,
in which R²⁶ represents hydrogen, (C₁-C₄)-alkyl, phenyl or naphthyl,
in which R²⁷ represents hydrogen,
and
in which phenyl and 5- or 6-membered heteroaryl may be substituted by 1 to 3 substituents independently of one another selected from the group consisting of fluorine, chlorine, bromine, trifluoromethyl, methyl and ethyl,
R¹⁷ represents hydrogen or (C₁-C₄)-alkyl,
or
R¹⁶ and R⁷ together with the carbon atom to which they are attached form a 3- to 5-membered carbocycle,
R¹⁸ represents hydrogen, (C₁-C₆)-alkyl or (C₃-C₅)-cycloalkyl, in which (C₁-C₄)-alkyl may be substituted by 1 to 5 fluorine substituents,
and
in which (C₃-C₅)-cycloalkyl may be substituted by 1 or 2 substituents independently of one another selected from the group consisting of fluorine, trifluoromethyl, hydroxy and (C₁-C₄)-alkyl,
R¹⁹ represents hydrogen or (C₁-C₄)-alkyl,
or
R¹⁸ and R¹⁹ together with the carbon atom to which they are attached form a 3- to 6-membered carbocycle,
in which the 3- to 6-membered carbocycle may be substituted by 1 or 2 fluorine or (C₁-C₄)-alkyl substituents,
or
R¹⁶ and R¹⁸ together with the carbon atoms to which they are attached form a 3- to 6-membered carbocycle or a 4- to 7-membered heterocycle,
with the proviso that not more than one of the R¹⁶ and R¹⁷, R¹⁸ and R¹⁹, and R¹⁶ and R¹⁸ radical pairs at the same time forms a carbo- or heterocycle,
R²⁰ represents hydrogen or (C₁-C₄)-alkyl,
in which (C₁-C₄)-alkyl may be substituted by 1 to 5 fluorine substituents,
m represents 0 or 1,
n represents 0 or 1,
R²¹ represents hydrogen, cyano or (C₁-C₄)-alkyl,
in which (C₁-C₄)-alkyl may be substituted by 1 to 5 fluorine substituents,
R²² represents hydrogen or (C₁-C₄)-alkyl,
in which (C₁-C₄)-alkyl may be substituted by 1 to 5 fluorine substituents,
R²³ represents hydrogen or (C₁-C₆)-alkyl,
in which (C₁-C₆)-alkyl may be substituted by 1 to 5 fluorine substituents,
R²⁴ represents hydrogen or (C₁-C₄)-alkyl,
in which (C₁-C₄)-alkyl may be substituted by 1 to 5 fluorine substituents,
or
R²¹ and R²² together with the carbon atom to which they are attached form a 3- to 5-membered carbocycle,
in which the 3- to 5-membered carbocycle may be substituted by 1 or 2 substituents independently of one another selected from the group consisting of fluorine, methyl, and ethyl,
or
R²³ and R²⁴ together with the carbon atom to which they are attached form a 3- to 5-membered carbocycle,
in which the 3- to 5-membered carbocycle may be substituted by 1 or 2 substituents independently of one another selected from the group consisting of fluorine, methyl and ethyl,
or
R²¹ and R²³ together with the carbon atom to which they are attached form a 3- to 5-membered carbocycle,
in which the 3- to 5-membered carbocycle may be substituted by 1 or 2 substituents independently of one another selected from the group consisting of fluorine, methyl and ethyl,
with the proviso that not more than one of the R²¹ and R²², R²³ and R²⁴, and R²¹ and R²³ radical pairs at the same time forms a carbocycle,
R²⁵ represents (C₁-C₆)-alkyl, 5- or 6-membered heterocyclyl attached via a ring carbon atom, 5- or 6-membered carbocyclyl, phenyl or 5- to 10-membered heteroaryl,
where (C₁-C₆)-alkyl may be substituted by cyano or up to three times by fluorine,
in which phenyl may be substituted by 1 to 3 substituents independently of one another selected from the group consisting of halogen, cyano, trifluoromethyl, difluoromethyl, (C₁-C₄)-alkyl, (C₁-C₄)-alkoxy and hydroxy,
in which phenyl may be substituted by 5- or 6-membered heteroaryl,
in which 5- or 6-membered heteroaryl may be substituted by 1 to 3 substituents independently of one another selected from the group consisting of fluorine, chlorine, cyano, (C₁-C₄)-alkyl, trifluoromethyl, (C₁-C₄)-alkoxy and amino,
in which (C₁-C₄)-alkyl may be substituted by 1 to 3 substituents independently of one another selected from the group consisting of fluorine, chlorine, bromine, cyano, hydroxy, amino, trifluoromethyl, difluoromethyl, (C₁-C₄)-alkoxy, trifluoromethoxy, difluoromethoxy and phenyl,
in which phenyl may be substituted by 1 to 3 substituents independently of one another selected from the group consisting of fluorine, chlorine, bromine, (C₁-C₄)-alkyl and (C₁-C₄)-alkoxy,
in which 5- or 6-membered heterocyclyl attached via a ring carbon atom may be substituted by 1 to 3 substituents independently of one another selected from the group consisting of oxo, fluorine, trifluoromethyl, hydroxy and (C₁-C₄)-alkyl,
in which 5- or 6-membered heterocyclyl attached via a ring carbon atom may be fused to a phenyl ring or a pyridyl ring, which for their part may be substituted by 1 to 3 substituents selected from the group consisting of fluorine, chlorine, bromine, (C₁-C₄)-alkyl and trifluoromethyl,
and
in which 5- or 6-membered carbocyclyl may be substituted by 1 or 2 substituents independently of one another selected from the group consisting of trifluoromethyl, fluorine, cyano, hydroxy, amino and methyl,
in which 5- or 6-membered carbocyclyl may be fused to a phenyl ring or a pyridyl ring, which for their part may be substituted by 1 to 3 substituents selected from the group consisting of fluorine, chlorine, bromine, methyl, ethyl, methoxy, ethoxy and trifluoromethyl,
R⁶ represents hydrogen,
R⁷ represents hydrogen, fluorine, chlorine, bromine, cyano, difluoromethyl, trifluoromethyl, (C₁-C₄)-alkyl, (C₂-C₄)-alkynyl or (C₃-C₅)-cycloalkyl,
R⁸ represents hydrogen or fluorine,
and the N-oxides, salts, solvates, salts of the N-oxides and solvates of the N-oxides and salts thereof.

3. A compound of the formula (I-A) as claimed in claim 1 in which
A represents CH₂,
R¹ represents phenyl,
where phenyl may be substituted by 1 to 4 substituents independently of one another selected from the group consisting of fluorine and chlorine,
R² represents hydrogen or methyl,
R³ represents hydrogen or methyl,
R⁴ represents hydrogen,
R⁵ represents a group of the formula 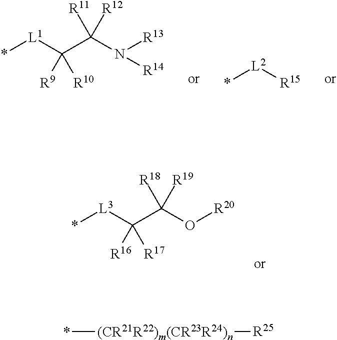
where
* represents the point of attachment to the amino group,
L¹ represents a bond or methanediyl,
L²represents a bond,
L³represents a bond, methanediyl or 1,2-ethanediyl,
R⁹ represents hydrogen,
R¹⁰ represents hydrogen,
R¹¹represents hydrogen, (C₁-C₄)-alkyl, cyclopropyl or cyclobutyl,
in which (C₁-C₄)-alkyl may be substituted by phenyl,
in which phenyl may be substituted by 1 or 2 substituents independently of one another selected from the group consisting of fluorine, chlorine and methoxy,
in which (C₁-C₄)-alkyl may be substituted by 1 to 5 fluorine substituents,
R¹² represents hydrogen or (C₁-C₄)-alkyl,
or
R¹¹ and R¹² together with the carbon atom to which they are attached form a 3- to 5-membered carbocycle,
R¹³ represents hydrogen, methyl or ethyl,
in which ethyl may be substituted by 1 to 3 fluorine substituents,
R¹⁴ represents hydrogen, methyl or cyclopropyl,
or
R¹³ and R¹⁴ together with the nitrogen atom to which they are attached form a morpholinyl ring or piperidinyl ring,
R¹⁵ represents 9-azabicyclo[3.3.1]nonan-3-yl or piperidin-4-yl,
in which 9-azabicyclo[3.3.1]nonan-3-yl is substituted by methyl,
in which piperidin-4-yl is substituted by 1 to 5 methyl substituents,
R¹⁶ represents hydrogen, (C₁-C₈)-alkyl, —(C═O)NR²⁶R²⁷ or phenyl,
in which (C₁-C₈)-alkyl may be substituted by a hydroxy or methoxy radical or up to five times by fluorine,
in which R²⁶ represents phenyl or naphthyl,
in which R²⁷ is hydrogen,
and
in which phenyl may be substituted by 1 to 3 substituents independently of one another selected from the group consisting of fluorine, chlorine and methyl,
R¹⁷ represents hydrogen or (C₁-C₄)-alkyl,
R¹⁸ represents hydrogen, (C₁-C₆)-alkyl or cyclopropyl,
in which (C₁-C₆)-alkyl may be substituted by 1 to 5 fluorine substituents,
R¹⁹ represents hydrogen or (C₁-C₄)-alkyl,
or
R¹⁸ and R¹⁹ together with the carbon atom to which they are attached form a 3- to 5-membered carbocycle,
R²⁰ represents hydrogen or (C₁-C₄)-alkyl,
in which (C₁-C₄)-alkyl may be substituted by 1 to 5 fluorine substituents,
m represents 0 or 1,
n represents 0 or 1,
R²¹ represents hydrogen, cyano or methyl,
in which methyl may be substituted by 1 to 3 fluorine substituents,
R²² represents hydrogen or methyl,
in which methyl may be substituted by 1 to 3 fluorine substituents,
R²³ represents hydrogen or methyl,
in which methyl may be substituted by 1 to 3 fluorine substituents,
R²⁴ represents hydrogen or methyl,
in which methyl may be substituted by 1 to 3 fluorine substituents,
or
R²¹ and R²² together with the carbon atom to which they are attached form a 3- to 5-membered carbocycle,
or
R²¹ and R²³ together with the carbon atom to which they are attached form a 3- to 5-membered carbocycle,
with the proviso that not more than one of the R²¹ and R²², and R²¹ and R²³ radical pairs at the same time forms a carbocycle,
R²⁵ represents (C₁-C₆)-alkyl, 2-oxopyrrolidin-3-yl, 2-oxotetrahydrofuran-3-yl, cyclopropyl, cyclobutyl, cyclopentyl, cyclohexyl, phenyl, 2,3-dihydro-1H-inden-1-yl, 3,4-dihydro-2H-pyrano[2,3-b]pyridin-4-yl, 1,2,3,4-tetrahydrochinolin-4-yl, 1,2,4-oxadiazol-5-yl, 1H-imidazol-2-yl, 1H-pyrazol-4-yl, pyridin-3-yl, pyrimidin-5-yl, quinolin-4-yl or pyrazolo[1,5-a]pyridin-3-yl,
in which (C₁-C₆)-alkyl may be up to trisubstituted by fluorine,
in which phenyl may be substituted by 1 or 2 substituents independently of one another selected from the group consisting of fluorine, chlorine, cyano, trifluoromethyl, methyl, ethyl and methoxy,
in which phenyl may be substituted by pyridyl or 1H-imidazol-1-yl,
in which 1,2,4-oxadiazol-5-yl, 1H-imidazol-2-yl, 1H-pyrazol-4-yl, pyridin-3-yl, pyrimidin-5-yl, 2,3-dihydro-1H-inden-1-yl, quinolin-4-yl or pyrazolo[1,5-a]pyridin-3-yl may be substituted by 1 to 3 substituents independently of one another selected from the group consisting of fluorine, chlorine, trifluoromethyl, (C₁-C₃)-alkyl, amino and hydroxyl,
in which (C₁-C₃)-alkyl may be substituted by fluorine, hydroxy, amino, phenyl or trifluoromethyl,
in which phenyl may be substituted by 1 or 2 substituents independently selected from the group consisting of fluorine and chlorine,
in which cyclopropyl, cyclobutyl, cyclopentyl and cyclohexyl are substituted by hydroxy,
R⁶ represents hydrogen,
R⁷ represents hydrogen or methyl,
R⁸ represents hydrogen,
and the N-oxides, salts, solvates, salts of the N-oxides and solvates of the N-oxides and salts thereof.

4. A compound of the formula (I-A) as claimed in claim 1, in which
A represents CH₂,
R¹ represents phenyl,
where phenyl may be substituted by 1 to 3 fluorine substituents,
R² represents hydrogen or methyl,
R³ represents hydrogen,
R⁴ represents hydrogen,
R⁵ represents a group of the formula 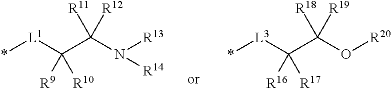
where
* represents the point of attachment to the amino group,
L¹ represents a bond,
L³represents a bond, methanediyl or 1,2-ethanediyl,
R⁹ represents hydrogen,
R¹⁰ represents hydrogen,
R¹¹ represents hydrogen, (C₁-C₈)-alkyl or cyclopropyl,
in which (C₁-C₈)-alkyl may be substituted by phenyl,
in which phenyl may be substituted by 1 or 2 substituents independently of one another selected from the group consisting of chlorine and methoxy,
in which (C₁-C₈)-alkyl may be substituted by 1 to 5 fluorine substituents,
R¹² represents hydrogen or (C₁-C₄)-alkyl,
or
R¹¹ and R¹² together with the carbon atom to which they are attached form a 3- to 5-membered carbocycle,
R¹³ represents hydrogen, methyl or ethyl,
in which ethyl may be substituted by 1 to 3 fluorine substituents,
R¹⁴ represents hydrogen, methyl or cyclopropyl,
or
R¹³ and R¹⁴ together with the nitrogen atom to which they are attached form a morpholinyl ring or piperidinyl ring,
R¹⁵ represents 9-azabicyclo[3.3.1]nonan-3-yl or piperidin-4-yl,
in which 9-azabicyclo[3.3.1]nonan-3-yl is substituted by methyl,
in which piperidin-4-yl is substituted by 1 to 5 methyl substituents,
R¹⁶ represents hydrogen, (C₁-C₈)-alkyl, —(C═O)NR²⁶R²⁷ or phenyl,
in which (C₁-C₈)-alkyl may be substituted by a hydroxy or methoxy radical or up to five times by fluorine,
in which R²⁶ represents phenyl or naphthyl,
in which R²⁷ represents hydrogen,
and
in which phenyl may be substituted by 1 to 3 substituents independently of one another selected from the group consisting of fluorine, chlorine and methyl,
R¹⁷ represents hydrogen or (C₁-C₄)-alkyl,
R¹⁸ represents hydrogen, (C₁-C₆)-alkyl or cyclopropyl,
R¹⁹ represents hydrogen or (C₁-C₄)-alkyl,
or
R¹⁸ and R¹⁹ together with the carbon atom to which they are attached form a 3- to 5-membered carbocycle,
R²⁰ represents hydrogen or (C₁-C₄)-alkyl,
in which (C₁-C₄)-alkyl may be substituted by 1 to 5 fluorine substituents,
R⁶ represents hydrogen,
R⁷ represents hydrogen or methyl,
R⁸ represents hydrogen,
and the N-oxides, salts, solvates, salts of the N-oxides and solvates of the N-oxides and salts thereof.

5. A compound of the formula (I-A) as claimed in claim 1 in which
A represents CH₂,
R¹ represents a phenyl group of the formula 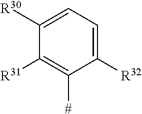
where
# represents the point of attachment to A,
and
R³ represents hydrogen or fluorine,
R³¹ represents fluorine,
R³² represents fluorine,
R² represents hydrogen or methyl,
R³ represents hydrogen,
R⁴ represents hydrogen,
R⁵ represents a group of the formula 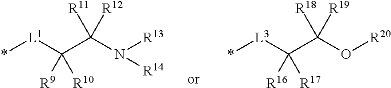
where
* represents the point of attachment to the amino group,
L¹ represents a bond,
L³represents a bond,
R⁹ represents hydrogen,
R¹⁰ represents hydrogen,
R¹¹ represents hydrogen or (C₁-C₆)-alkyl,
in which (C₁-C₆)-alkyl may be substituted by 1 to 5 fluorine substituents,
R¹² represents hydrogen, methyl or ethyl,
R¹³ represents hydrogen,
R¹⁴ represents hydrogen,
R¹⁶ represents hydrogen, (C₁-C₆)-alkyl, —(C═O)NR²⁶R²⁷ or phenyl,
in which (C₁-C₆)-alkyl may be substituted by a hydroxy or methoxy radical or up to five times by fluorine,
in which R²⁶ represents naphthyl,
in which R²⁷ represents hydrogen,
and
in which phenyl may be substituted by fluorine,
R¹⁷ represents hydrogen, methyl or ethyl,
R¹⁸ represents hydrogen, methyl or ethyl,
R¹⁹ represents hydrogen, methyl or ethyl,
or
R¹⁸ and R¹⁹ together with the carbon atom to which they are attached form a cyclopropyl ring,
R²⁰ represents hydrogen,
R⁶ represents hydrogen,
R⁷ represents hydrogen or methyl,
R⁸ represents hydrogen,
and the N-oxides, salts, solvates, salts of the N-oxides and solvates of the N-oxides and salts thereof.

6. A process for preparing compounds of the formula (I-A) or (I-B) as defined in claim 1, wherein
a compound of the formula (II-A) or (II-B) 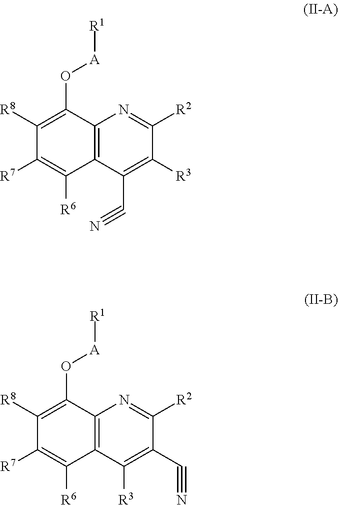
in which A, R¹, R², R³, R⁶, R⁷ and R⁸ each have the meanings given above,
is reacted in an inert solvent in the presence of a suitable base or acid to give a carboxylic acid of the formula (III-A) or (III-B) 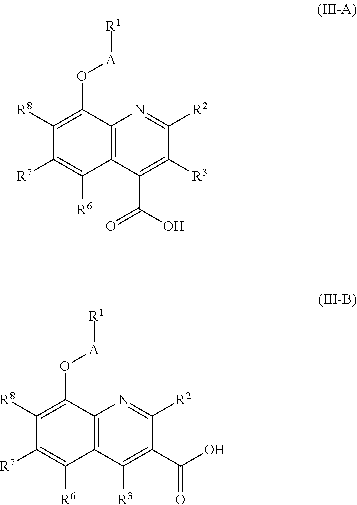
in which A, R¹, R², R³, R⁶, R⁷ and R⁸ each have the meanings given above,
and this is subsequently reacted in an inert solvent under amide coupling conditions with an amine of the formula (IV) 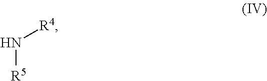
in which R⁴ and R⁵ each have the meanings given above,
then any protective groups present are detached, and the resulting compounds of the formula (I) are optionally converted with the appropriate (i) solvents and/or (ii) acids or bases to the solvates, salts and/or solvates of the salts thereof.

7. (canceled)

8. (canceled)

9. A medicament comprising a compound of the formula (I-A) or (I-B) as defined in claim 1 in combination with an inert, non-toxic, pharmaceutically suitable excipient.

10. A medicament comprising a compound of the formula (I-A) or (I-B) as defined in claim 1 in combination with a further active compound selected from the group consisting of organic nitrates, NO donors, cGMP-PDE inhibitors, antithrombotic agents, hypotensive agents and lipid metabolism modifiers.

11. (canceled)

12. A method for the treatment and/or prophylaxis of heart failure, angina pectoris, hypertension, pulmonary hypertension, ischemias, vascular disorders, renal insufficiency, thromboembolic disorders and arteriosclerosis in humans and animals comprising administering to the human or animal in need thereof an effective amount of at least one compound of the formula (I-A) or (I-B) as defined in claim 1.

13. A method for the treatment and/or prophylaxis of heart failure, angina pectoris, hypertension, pulmonary hypertension, ischemias, vascular disorders, renal insufficiency, thromboembolic disorders and arteriosclerosis in humans and animals comprising administering to the human or animal in need thereof an effective amount of the medicament of claim 9.
